(19) United States
(12) Patent Application Publication
(10) Pub. No.: US 2009/0042926 A1
(43) Pub. Date: Feb. 12, 2009

(54) Niacin Receptor Agonists, Compositions Containing Such Compounds and Methods of Treatment

(76) Inventors: Jason Imbriglio, Piscataway, NJ (US); Steven L. Colletti, Princeton Junction, NJ (US); James R. Tata, Westfield, NJ (US); Richard T. Beresis, Matawan, NJ (US); Daria Marley, Raleigh, NC (US); Subharekha Raghavan, Teaneck, NJ (US); Darby Rye Schmidt, Clark, NJ (US); Ashley Rouse Lins, Edison, NJ (US); Abigail L. Smenton, Brooklyn, NJ (US); Weichun Chen, Livingston, NJ (US); Hong Shen, West Windsor, NJ (US); Fa-Xiang Ding, Staten Island, NY (US); Rena Bodner, New York, NY (US)

Correspondence Address: MERCK AND CO., INC, P O BOX 2000, RAHWAY, NJ, 07065-0907, US

(21) Appl. No.: 12/086,938

(22) PCT Filed: Dec. 20, 2006
(86) PCT No.: PCT/US2006/048535

§ 371 Date: Jun. 20, 2008

Related U.S. Application Data
(60) Provisional application No. 60/751,877, filed on Dec. 20, 2005.

(51) Int. Cl. A61K 31/4439; C07C 229/34; C07D 277/22; C07D 271/06; C07D 413/04; A61K 31/437; A61P 9/10; A61P 3/10; C07D 471/04; A61K 31/196; A61K 31/426; A61K 31/4245

(52) U.S. Cl. 514/293; 562/457; 548/204; 548/131; 546/269.4; 546/86; 514/563; 514/365; 514/364; 514/340

(57) ABSTRACT

The present invention encompasses compounds of Formula (I): as well as pharmaceutically acceptable salts and hydrates thereof, that are useful for treating atherosclerosis, dyslipidemias and the like. Pharmaceutical compositions and methods of use are also included.

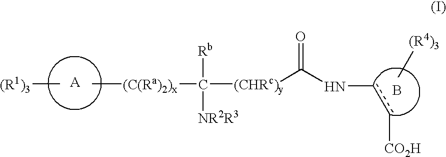

### BACKGROUND OF THE INVENTION

The present invention relates to amino-substituted compounds, their derivatives, compositions containing such compounds and methods of treatment or prevention in a mammal relating to dyslipidemias. Dyslipidemia is a condition wherein serum lipids are abnormal. Elevated cholesterol and low levels of high density lipoprotein (HDL) are independent risk factors for atherosclerosis associated with a greater-than-normal risk of atherosclerosis and cardiovascular disease. Factors known to affect serum cholesterol include genetic predisposition, diet, body weight, degree of physical activity, age and gender. While cholesterol in normal amounts is a vital building block for cell membranes and essential organic molecules such as steroids and bile acids, cholesterol in excess is known to contribute to cardiovascular disease. For example, cholesterol, through its relationship with foam cells, is a primary component of plaque which collects in coronary arteries, resulting in the cardiovascular disease termed atherosclerosis.

Traditional therapies for reducing cholesterol include medications such as statins (which reduce production of cholesterol by the body). More recently, the value of nutrition and nutritional supplements in reducing blood cholesterol has received significant attention. For example, dietary compounds such as soluble fiber, vitamin E, soy, garlic, omega-3 fatty acids, and niacin have all received significant attention and research funding.

Niacin or nicotinic acid (pyridine-3-carboxylic acid) is a drug that reduces coronary events in clinical trials. It is commonly known for its effect in elevating serum levels of high density lipoproteins (HDL). Importantly, niacin also has a beneficial effect on other lipid profiles. Specifically, it reduces low density lipoproteins (LDL), very low density lipoproteins (VLDL), and triglycerides (TG). However, the clinical use of nicotinic acid is limited by a number of adverse side-effects including cutaneous vasodilation, sometimes called flushing.

Despite the attention focused on traditional and alternative means for controlling serum cholesterol, serum triglycerides, and the like, a significant portion of the population has total cholesterol levels greater than about 200 mg/dL, and are thus candidates for dyslipidemia therapy. There thus remains a need in the art for compounds, compositions and alternative methods of reducing total cholesterol, serum triglycerides, and the like, and raising HDL.

The present invention relates to compounds that have been discovered to have effects in modifying serum lipid levels.

The invention thus provides compositions for effecting reduction in total cholesterol and triglyceride concentrations and raising HDL, in accordance with the methods described.

Consequently one object of the present invention is to provide a nicotinic acid receptor agonist that can be used to treat dyslipidemias, atherosclerosis, diabetes, metabolic syndrome and related conditions while minimizing the adverse effects that are associated with niacin treatment.

Yet another object is to provide a pharmaceutical composition for oral use.

These and other objects will be apparent from the description provided herein.

### SUMMARY OF THE INVENTION

A compound represented by formula I:

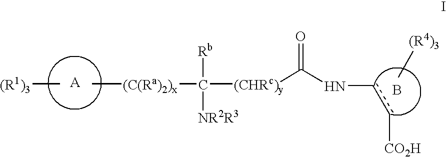

or a pharmaceutically acceptable salt, solvate or ester thereof is disclosed wherein:

ring A represents a 6-10 membered aryl, a 5-13 membered heteroaryl or a non-aromatic or partially aromatic heterocyclic group, said heteroaryl and non-aromatic and partially aromatic heterocyclic groups containing at least one heteroatom selected from O, S, S(O), S(O)₂ and N, and optionally containing 1 other heteroatom selected from O and S, and optionally containing 1-3 additional N atoms, with up to 5 heteroatoms being present;

ring B represents a phenyl, thiophene or a cyclohexenyl ring in which the dotted line and the line which it is adjacent to represent in combination a double bond;

each R¹ is H or is independently selected from the group consisting of:

a) halo, OH, CO₂H, CN, NH₂, S(O)₀₋₂R^{e}, C(O)R^{e}, OC(O)R^{e} and CO₂R^{e}, wherein R^{e} represents C₁₋₄alkyl or phenyl, said C₁₋₄alkyl and phenyl each being optionally substituted with 1-3 groups, 1-3 of which are selected from halo and C₁₋₃alkyl, and 1-2 of which are selected from the group consisting of: OC₁₋₃alkyl, haloC₁₋₃alkyl, haloC₁₋₃alkoxy, OH, NH₂ and NHC₁₋₃alkyl;

b) C₁₋₆ alkyl and OC₁₋₆alkyl, said C₁₋₆alkyl and alkyl portion of OC₁₋₆alkyl being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy and CN;

c) NHC₁₋₄alkyl and N(C₁₋₄alkyl)₂, the alkyl portions of which are optionally substituted as set forth in (b) above;

d) C(O)NH₂, C(O)NHC₁₋₄alkyl, C(O)N(C₁₋₄alkyl)₂, C(O)Hetcy, C(O)NHOC₁₋₄alkyl and C(O)N(C₁₋₄alkyl)(OC₁₋₄alkyl), the alk portions of which are optionally substituted as set forth in (b) above;

e) NR′C(O)R″, NR′SO₂R″, NR′CO₂R″ and NR′C(O)NR″R′″ wherein:

R′ represents H, C₁₋₃alkyl or haloC₁₋₃alkyl,

R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄haloallyl, NH₂, NHC₁₋₄alkyl, N(C₁₋₄alkyl)₂, CN, Hetcy, Aryl and HAR,
said Hetcy, Aryl and HAR being further optionally substituted with 1-3 halo, C₁₋₄alkyl, C₁₋₄alkoxy, haloC₁₋₄-alkyl or haloC₁₋₄alkoxy groups; and
   (b) Hetcy, Aryl or HAR, each being optionally substituted with 1-3 members selected from the group consisting of: halo, C₁₋₄alkyl, C₁₋₄alkoxy, haloC₁₋₄alkyl and haloC₁₋₄alkoxy groups;

and R′″ representing H or R″;

f) phenyl or a 5-6 membered heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH; CO₂H; CN; NH₂ and S(O)O₂R^{e} wherein R^{e} is as described above;
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, NH₂, NHC₁₋₄alkyl, N(C₁₋₄alkyl)₂ and CN;
iii) C(O)NH₂, C(O)NHC₁₋₄alkyl, C(O)N(C₁₋₄alkyl)₂, C(O)NMOC₁₋₄alkyl and C(O)N(C₁₋₄alkyl)(OC₁₋₄alkyl), the alkyl portions of which are optionally substituted as set forth in b) above; and
iv) NR′C(O)R″, NR′SO₂R″, NR′CO₂R″ and NR′C(O)NR″R′″ wherein R′, R″ and R′″ are as described above;

one of x and y is 0 and the other is 1;

each R^{a}, R^{b} and R^{c} are selected from H, C₁₋₃alkyl and haloC₁₋₃alkyl;

R² and R³ represent H, C₁₋₃alkyl or haloC₁₋₃alkyl;

3 R⁴ groups are present, 0-1 of which represents Aryl, HAR or Hetcy, said Aryl, HAR or Hetcy group being optionally substituted with up to 3 groups, 1-3 of which are halo, and 0-1 of which are selected from the group consisting of: OH, NH₂, C₁₋₃alkyl, C₁₋₃alkoxy, baloC₁₋₃alkyl and haloC₁₋₃alkoxy;

and the remainder of the R⁴groups are selected from the group consisting of: H, halo, C₁₋₃alkyl, C₁₋₃alkoxy, OH, NH₂, NHC₁₋₃alkyl, N(C₁₋₃alkyl)₂ and CN, said alkyl and alkyl portions of C₁₋₃alkoxy, NHC₁₋₃alkyl and N(C₁₋₃alkyl)₂ being optionally substituted with 1-3 groups, 0-3 of which are halo, and 0-1 of which are selected from the group consisting of: OC₁₋₃alkyl, OH, NH₂, NHC₁₋₃alkyl, N(C₁₋₃alkyl)₂, CN, Hetcy, Aryl and HAR,

said Aryl and HAR being further optionally substituted with 1-3 groups, 0-3 of which are halo, and 0-1 of which are selected from the group consisting of: OH, NH₂, C₁₋₃alk-yl, C₁₋₃alkoxy, haloC₁₋₃alkyl and haloC₁₋₃alkoxy groups.

### DETAILED DESCRIPTION OF THE INVENTION

The invention is described herein in detail using the terms defined below unless otherwise specified.

“Alkyl”, as well as other groups having the prefix “alk”, such as alkoxy, alkanoyl and the like, means carbon chains which may be linear, branched, or cyclic, or combinations thereof, containing the indicated number of carbon atoms. If no number is specified, 1-6 carbon atoms are intended for linear and 3-7 carbon atoms for branched alkyl groups. Examples of alkyl groups include methyl, ethyl, propyl, isopropyl, butyl, sec- and tert-butyl, pentyl, hexyl, heptyl, octyl, nonyl and the like. Cycloalkyl is a subset of alkyl; if no number of atoms is specified, 3-7 carbon atoms are intended, forming 1-3 carbocyclic rings that are fused. “Cycloalkyl” also includes monocyclic rings fused to an aryl group in which the point of attachment is on the non-aromatic portion. Examples of cycloalkyl include cyclopropyl, cyclobutyl, cyclopentyl, cyclohexyl, cycloheptyl, tetrahydronaphthyl, decahydronaphthyl, indanyl and the like.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Aryl” (Ar) means mono- and bicyclic aromatic rings containing 6-10 carbon atoms. Examples of aryl include phenyl, naphthyl, indenyl and the like.

“Heteroaryl” (HAR) unless otherwise specified, means mono-, bicyclic and tricyclic aromatic ring systems containing at least one heteroatom selected from O, S, S(O), SO₂ and N, with each ring containing 5 to 6 atoms. HAR groups may contain from 5-14, preferably 5-13 atoms. Examples include, but are not limited to, pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzoxazolyl, benzothiazolyl, benzimidazolyl, benzofuranyl, benzothiophenyl, benzopyrazolyl, benzotriazolyl, furo(2,3-b)pyridyl, benzoxazinyl, tetrahydrohydroquinolinyl, tetrahydroisoquinolinyl, quinolyl, isoquinolyl, indolyl, dihydroindolyl, quinoxalinyl, quinazolinyl, naphthyridinyl, pteridinyl, 2,3-dihydrofuro(2,3-b)pyridyl and the like. Heteroaryl also includes aromatic carbocyclic or heterocyclic groups fused to heterocycles that are non-aromatic or partially aromatic, and optionally containing a carbonyl. Examples of additional heteroaryl groups include indolinyl, dihydrobenzofuranyl, dihydrobenzothiophenyl, dihydrobenzoxazolyl, and aromatic heterocyclic groups fused to cycloalkyl rings. Examples also include the following:

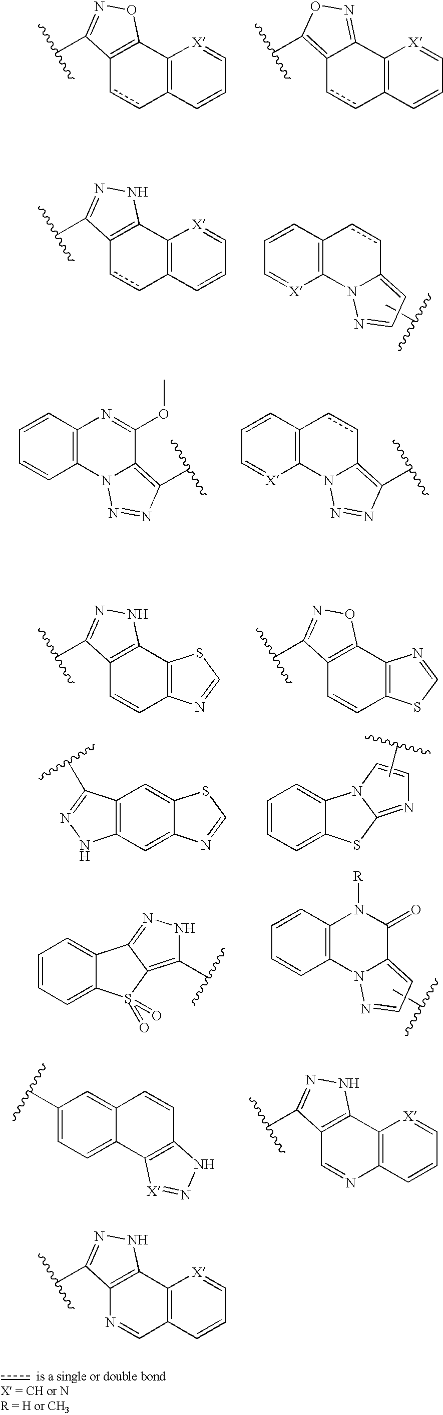

Heteroaryl also includes such groups in charged form, e.g., pyridinium.

“Heterocyclyl” (Hetcy) unless otherwise specified, means mono- and bicyclic saturated rings and ring systems containing at least one heteroatom selected from N, S and O, each of said ring having from 3 to 10 atoms in which the point of attachment may be carbon or nitrogen. Examples of “heterocyclyl” include, but are not limited to, azetidinyl, pyrrolidinyl, piperidinyl, piperazinyl, imidazolidinyl, tetrahydrofuranyl, 1,4-dioxanyl, morpholinyl, thiomorpholinyl, tetrahydrothienyl and the like. Heterocycles can also exist in tautomeric forms, e.g., 2- and 4-pyridones. Heterocycles moreover includes such moieties in charged form, e.g., piperidinium.

“Halogen” (Halo) includes fluorine, chlorine, bromine and iodine.

The phrase “in the absence of substantial flushing” refers to the side effect that is often seen when nicotinic acid is administered in therapeutic amounts. The flushing effect of nicotinic acid usually becomes less frequent and less severe as the patient develops tolerance to the drug at therapeutic doses, but the flushing effect still occurs to some extent and can be transient. Thus, “in the absence of substantial flushing” refers to the reduced severity of flushing when it occurs, or fewer flushing events than would otherwise occur. Preferably, the incidence of flushing (relative to niacin) is reduced by at least about a third, more preferably the incidence is reduced by half, and most preferably, the flushing incidence is reduced by about two thirds or more. Likewise, the severity (relative to niacin) is preferably reduced by at least about a third, more preferably by at least half, and most preferably by at least about two thirds. Clearly a one hundred percent reduction in flushing incidence and severity is most preferable, but is not required.

An aspect of the invention that is of interest relates to a compound represented by formula I:

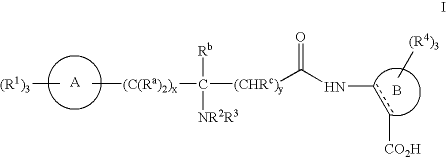

or a pharmaceutically acceptable salt, solvate or ester thereof is disclosed wherein:
ring A represents a 6-10 membered aryl, a 5-13 membered heteroaryl or a non-aromatic or partially aromatic heterocyclic group, said heteroaryl and non-aromatic and partially aromatic heterocyclic groups containing at least one heteroatom selected from O, S, S(O), S(O)₂ and N, and optionally containing 1 other heteroatom selected from O and S, and optionally containing 1-3 additional N atoms, with up to 5 heteroatoms being present;

ring B represents a phenyl, thiophene or a cyclohexenyl ring in which the dotted line and the line which it is adjacent to represent in combination a double bond;

each R¹ is H or is independently selected from the group consisting of:

a) halo, OH, CO₂H, CN, NH₂, S(O)₀₋₂R^{e}, C(O)R^{e}, OC(O)Rₑ and CO₂RC^{e}, wherein R^{e} represents C₁₋₄alkyl or phenyl, said C₁₋₄alkyl and phenyl each being optionally substituted with 1-3 groups, 1-3 of which are selected from halo and C₁₋₃alkyl, and 1-2 of which are selected from the group consisting of: OC₁₋₃alklyl, haloC₁₋₃alkyl, haloC₁₋₃alkoxy, OH, NH₂ and NHC₁₋₃alkyl;

b) C₁₋₆ alkyl and OC₁₋₆alkyl, said C₁₋₆alkyl and alkyl portion of OC₁₋₆-alkyl being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy and CN;

c) NHC₁₋₄alkyl and N(C₁₋₄alkyl)₂, the alkyl portions of which are optionally substituted as set forth in (b) above;

d) C(O)NH₂, C(O)NHIC₄alkyl, C(O)N(C₁₋₄alkyl)₂, C(O)Hetcy, C(O)NHOC₁₋₄alkyl and C(O)N(C₁₋₄alkyl)(OC₁₋₄alkyl), the alkyl portions of which are optionally substituted as set forth in (b) above;

e) NR′C(O)R″, NR′SO₂R″, NR′CO₂R″ and NR′C(O)NR″R′″ wherein:

R′ represents H, C₁₋₃alkyl or haloC₁₋₃alkyl,

R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄haloalkyl, NH₂, NHCl₄alkyl, N(C₁₋₄alkyl)₂, CN, Hetcy, Aryl and HAR,
said Hetcy, Aryl and HAR being further optionally substituted with 1-3 halo, C₁₋₄alkyl, C₁₋₄alkoxy, haloC₁₋₄alkyl or haloC₁₋₄alkoxy groups; and
   (b) Hetcy, Aryl or HAR, each being optionally substituted with 1-3 members selected from the group consisting of: halo, C₁₋₄alkyl, C₁₋₄alkoxy, haloC₁₋₄alkyl and haloC₁₋₄alkoxy groups;

and R′″ representing H or R″;

f) phenyl or a 5-6 membered heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH; CO₂H; CN; NH₂ and S(O)₀₋₂R^{e} wherein R^{e} is as described above;
ii) NHC₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, NH₂, NHC₁₋₄alkyl, N(C₁₋₄alkyl)₂ and CN;
iii) C(O)NH₂, C(O)NHC₁₋₄alkyl, C(O)N(C₁₋₄alkyl)₂, C(O)NHOC₁₋₄alkyl and C(O)N(C₁₋₄alkyl)(OC₁₋₄alkyl), the alkyl portions of which are optionally substituted as set forth in b) above; and
iv) NR′C(O)R″, NR′SO₂R″, NR′CO₂R″ and NR′C(O)NR″R′″ wherein R′, R″ and R′″ are as described above;

one of x and y is 0 and the other is 1;

each R^{a}, R^{b} and R^{c} are selected from H, C₁₋₃alkyl and haloC₁₋₃alkyl;

R² and R³ represent H, C₁₋₃alkyl or haloC₁₋₃alkyl;

3 R⁴ groups are present, 0-1 of which represents Aryl, HAR or Hetcy, said Aryl, HAR or Hetcy group being optionally substituted with up to 3 groups, 1-3 of which are halo, and 0-1 of which are selected from the group consisting of: OH, NH₂, C₁₋₃alkyl, C₁₋₃alkoxy, haloC₁₋₃alkyl and haloC₁₋₃alkoxy;

and the remainder of the R⁴groups are selected from the group consisting of: H, halo, C₁₋₃alkyl, C₁₋₃alkoxy, OH, NH₂, NHC₁₋₃alkyl, N(C₁₋₃alkyl)₂ and CN, said alkyl and alkyl portions of C₁₋₃alkoxy, NHC₁₋₃alkyl and N(C₁₋₃alkyl)₂ being optionally substituted with 1-3 groups, 0-3 of which are halo, and 0-1 of which are selected from the group consisting of: OC₁₋₃alkyl, OH, NH₂, NHC₁₋₃alkyl, N(C₁₋₃alkyl)₂, CN, Hetcy, Aryl and HAR,

said Aryl and HAR being further optionally substituted with 1-3 groups, 0-3 of which are halo, and 0-1 of which are selected from the group consisting of: OH, NH₂, C₁₋₃alkyl, C₁₋₃alkoxy, haloC₁₋₃alkyl and haloC₁₋₃alkoxy groups.

Another aspect of the invention relates to a compound represented by formula Ia:

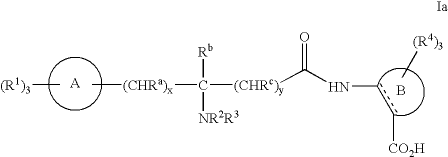

or a pharmaceutically acceptable salt, solvate or ester thereof is disclosed wherein:

ring A represents a 6-10 membered aryl, a 5-13 membered heteroaryl or a non-aromatic or partially aromatic heterocyclic group, said heteroaryl and non-aromatic and partially aromatic heterocyclic groups containing at least one heteroatom selected from O, S, S(O), S(O)₂ and N, and optionally containing 1 other heteroatom selected from O and S, and optionally containing 1-3 additional N atoms, with up to 5 heteroatoms being present;

ring B represents a phenyl, thiophene or a cyclohexenyl ring in which the dotted line and the line which it is adjacent to represent in combination a double bond;

each R¹ is H or is independently selected from the group consisting of:

a) halo, OH, CO₂H, CN, NH₂, S(O)O₂R^{e}, C(O)R^{e}, OC(O)R^{e} and CO₂R^{e}, wherein R^{e} represents C₁₋₄alkyl or phenyl, said C₁₋₄alkyl and phenyl each being optionally substituted with 1-3 groups, 1-3 of which are selected from halo and C₁₋₃alkyl, and 1-2 of which are selected from the group consisting of: OC₁₋₃alkyl, haloC₁₋₃alkyl, haloC₁₋₃alkoxy, OH, NH₂ and NHC₁₋₃alkyl;

b) C₁₋₆ alkyl and OC₁₋₆alkyl, said C₁₋₆alkyl and alkyl portion of OC₁₋₆alkyl being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy and CN;

c) NHC₁₋₄alkyl and N(C₁₋₄alkyl)₂, the alkyl portions of which are optionally substituted as set forth in (b) above;

d) C(O)NH₂, C(O)NHClAalkyl, C(O)N(C₁₋₄alkyl)₂, C(O)Hetcy, C(O)NHOC₁₋₄alkyl and C(O)N(C₁₋₄alkyl)(OC₁₋₄alkyl), the alkyl portions of which are optionally substituted as set forth in (b) above;

e) NR′C(O)R″, NR′SO₂R″, NR′CO₂R″ and NR′C(O)NR″R′″ wherein:

R′ represents H, C₁₋₃alkyl or haloC₁₋₃alkyl,

R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄haloalkyl, NH₂, NHC₁₋₄alkyl, N(C₁₋₄alkyl)₂, CN, Hetcy, Aryl and HAR,
said Hetcy, Aryl and HAR being further optionally substituted with 1-3 halo, C₁₋₄alkyl, C₁₋₄alkoxy, haloC₁₋₄alkyl or haloC₁₋₄alkoxy groups; and
   (b) Hetcy, Aryl or HAR, each being optionally substituted with 1-3 members selected from the group consisting of: halo, C₁₋₄alkyl, C₁₋₄alkoxy, haloC₁₋₄alkyl and haloC₁₋₄alkoxy groups;

and R′″ representing H or R″;

f) phenyl or a 5-6 membered heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH; CO₂H; CN; NH₂ and S(O)O₂R^{e} wherein R^{a} is as described above;
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, NH₂, NHC₁₋₄-alkyl, N(C₁₋₄alkyl)₂ and CN;
iii) C(O)NH₂, C(O)NHC₁₋₄alkyl, C(O)N(C₁₋₄alkyl)₂, C(O)NHOC₁₋₄alkyl and C(O)N(C₁₋₄alkyl)(OC₁₋₄alkyl), the alkyl portions of which are optionally substituted as set forth in b) above; and
iv) NR′C(O)R″, NR′SO₂R″, NR′CO₂R″ and NR′C(O)NR″R′″ wherein R′, R″ and R′″ are as described above;

one of x and y is 0 and the other is 1;

R^{a}, R^{b} and R^{c} are selected from H, C₁₋₃alkyl and haloC₁₋₃alkyl;

R² and R³ represent H, C₁₋₃alkyl or haloC₁₋₃alkyl;

3 R⁴ groups are present, 0-1 of which represents Aryl, HAR or Hetcy, said Aryl, HAR or Hetcy group being optionally substituted with up to 3 groups, 1-3 of which are halo, and 0-1 of which are selected from the group consisting of: OH, NH₂, C₁₋₃alkyl, C₁₋₃alkoxy, haloC₁₋₃alkyl and haloC₁₋₃alkoxy;

and the remainder of the R⁴groups are selected from the group consisting of: H, halo, C₁₋₃alkyl, C₁₋₃alkoxy, OH, NH₂, NHC₁₋₃alkyl, N(C₁₋₃alkyl)₂ and CN, said alkyl and alkyl portions of C₁₋₃alkoxy, NHC₁₋₃alkyl and N(C₁₋₃alkyl)₂ being optionally substituted with 1-3 groups, 0-3 of which are halo, and 0-1 of which are selected from the group consisting of: OC₁₋₃alkyl, OH, NH₂, NHC₁₋₃alkyl, N(C₁₋₃alkyl)₂, CN, Hetcy, Aryl and HAR,

said Aryl and HAR being further optionally substituted with 1-3 groups, 0-3 of which are halo, and 0-1 of which are selected from the group consisting of: OH, NH₂, C₁₋₃alkyl, C₁₋₃alkoxy, haloC₁₋₃alkyl and haloC₁₋₃alkoxy groups.

One subset of compounds that is of interest relates to compounds of formula I or Ia, or a pharmaceutically acceptable salt or solvate thereof, wherein ring A represents a 6-10 membered Aryl group. Within this subset of the invention, all other variables are as originally defined with respect to formula I.

Another subset of compounds that is of interest relates to compounds of formula I or Ia, or a pharmaceutically acceptable salt or solvate thereof, wherein Ring A represents a 5-13 membered heteroaryl (HAR) or heterocyclyl (Hetcy) group. Within this subset of the invention, all other variables are as originally defined with respect to formula I.

More particularly, a subset of compounds that is of interest relates to compounds of formula I, or a pharmaceutically acceptable salt or solvate thereof, wherein Ring A represents a 5 membered heteroaryl (HAR) group having 1 heteroatom selected from oxygen, sulfur and nitrogen, and 0-2 additional nitrogen atoms. Within this subset of the invention, all other variables are as originally defined with respect to formula I.

Even more particularly, another subset of compounds that is of interest relates to compounds of formula I or Ia, or a pharmaceutically acceptable salt or solvate thereof, wherein Ring A represents a 5 membered heteroaryl (HAR) group having 1 oxygen atom and 0-2 nitrogen atoms. Within this subset of the invention, all other variables are as originally defined with respect to formula I.

Even more particularly, another subset of compounds that is of interest relates to compounds of formula I or Ia, or a pharmaceutically acceptable salt or solvate thereof, wherein Ring A represents a 5 membered heteroaryl (HAR) group having 1 sulfur atom and 0-2 nitrogen atoms. Within this subset of the invention, all other variables are as originally defined with respect to formula I.

Even more particularly, another subset of compounds that is of interest relates to compounds of formula I or Ia, or a pharmaceutically acceptable salt or solvate thereof, wherein Ring A represents a 5 membered heteroaryl (HAR) group having 2-3 nitrogen atoms. Within this subset of the invention, all other variables are as originally defined with respect to formula I.

Still more particularly, another subset of compounds that is of interest relates to compounds of formula I or Ia, or a pharmaceutically acceptable salt or solvate thereof, wherein Ring A is selected from the group consisting of pyrazole, isoxazole, oxadiazole, triazole and thiazole. Within this subset of the invention, all other variables are as originally defined with respect to formula I.

A subset of compounds that is of interest relates to a compound of formula I or Ia, or a pharmaceutically acceptable salt or solvate thereof, wherein ring A is selected from the group consisting of oxazole, oxadiazole and pyrazole. Within this subset of the invention, all other variables are as originally defined with respect to formula I.

Additionally, a subset of compounds that is of interest relates to compounds of formula I or Ia, or a pharmaceutically acceptable salt or solvate thereof, wherein Ring A represents a tricyclic heteroaryl (HAR) group having 1-2 heteroatoms selected from oxygen, sulfur and nitrogen, and 0-3 additional nitrogen atoms. Within this subset of the invention, all other variables are as originally defined with respect to formula I.

Still more particularly, another subset of compounds that is of interest relates to compounds of formula I or Ia, or a pharmaceutically acceptable salt or solvate thereof, wherein Ring A represents a tricyclic heteroaryl (HAR) moiety selected from the following group:

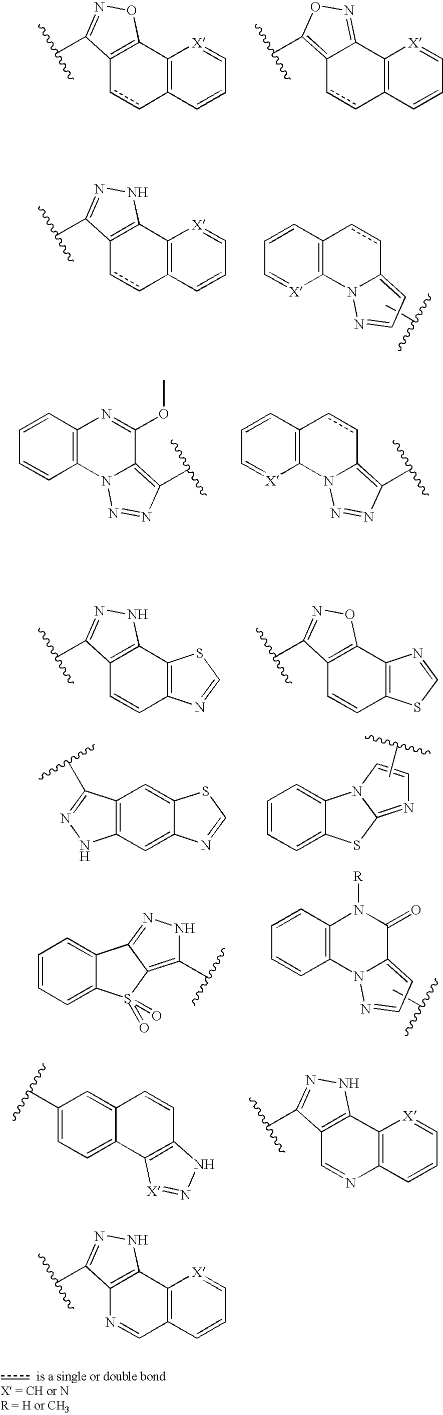

Within this subset of the invention, all other variables are as originally defined with respect to formula I.

Another subset of compounds that is of interest relates to compounds of formula I or Ia, or a pharmaceutically acceptable salt or solvate thereof, wherein ring B represents cyclohexenyl or phenyl. Within this subset of the invention, all other variables are as originally defined with respect to formula I.

Another subset of compounds that is of interest relates to compounds of formula I or Ia, or a pharmaceutically acceptable salt or solvate thereof, wherein ring B represents a phenyl ring. Within this subset of the invention, all other variables are as originally defined with respect to formula I.

Another subset of compounds that is of interest relates to compounds of formula I or Ia, or a pharmaceutically acceptable salt or solvate thereof, wherein ring B represents a thiophene ring. Within this subset of the invention, all other variables are as originally defined with respect to formula I.

Another subset of compounds that is of interest relates to compounds of formula I or Ia, or a pharmaceutically acceptable salt or solvate thereof, wherein ring B represents a cyclohexenyl ring. Within this subset of the invention, all other variables are as originally defined with respect to formula I.

Another subset of compounds that is of interest relates to compounds of formula I or Ia, or a pharmaceutically acceptable salt or solvate thereof, wherein x represents 1 and y represents 0. Within this subset of the invention, all other variables are as originally defined with respect to formula I.

Another subset of compounds that is of interest relates to compounds of formula I or a pharmaceutically acceptable salt or solvate thereof, wherein the moiety (C(R)₂), represents a —CH₂— or a —CH(CH₃)— group. Within this subset of the invention, all other variables are as originally defined with respect to formula I.

Another subset of compounds that is of interest relates to compounds of formula I or Ia, or a pharmaceutically acceptable salt or solvate thereof, wherein R^{b} represents H or CH₃. Within this subset of the invention, all other variables are as originally defined with respect to formula I.

Another subset of compounds that is of interest relates to compounds of formula I or Ia, or a pharmaceutically acceptable salt or solvate thereof, wherein x represents 0 and y represents 1. Within this subset of the invention, all other variables are as originally defined with respect to formula I.

More particularly, another subset of compounds that is of interest relates to compounds of formula I or Ia, or a pharmaceutically acceptable salt or solvate thereof, wherein x represents 1 and y represents 0, and R^{a} and R^{b} each represent H or methyl. Within this subset of the invention, all other variables are as originally defined with respect to formula I.

Additionally, another subset of compounds that is of interest relates to compounds of formula I or Ia, or a pharmaceutically acceptable salt or solvate thereof, wherein x represents 0 and y represents 1, and R^{b} and R^{c} each represent H or methyl. Within this subset of the invention, all other variables are as originally defined with respect to formula I.

Another subset of compounds that is of interest relates to compounds of formula I or Ia, or a pharmaceutically acceptable salt or solvate thereof, wherein R² and R³ represent H or CH₃. Within this subset of the invention, all other variables are as originally defined with respect to formula I.

Another subset of compounds that is of interest relates to compounds of formula I or Ia, or a pharmaceutically acceptable salt or solvate thereof, wherein R² and R³ represent hydrogen. Within this subset of the invention, all other variables are as originally defined with respect to formula I.

Another subset of compounds that is of interest relates to compounds of formula I or Ia, or a pharmaceutically acceptable salt or solvate thereof, wherein all R⁴ groups represent hydrogen. Within this subset of the invention, all other variables are as originally defined with respect to formula I.

Another subset of compounds that is of interest relates to compounds of formula I or Ia, or a pharmaceutically acceptable salt or solvate thereof, wherein each R⁴ is H or is selected from the group consisting of: CH₃, phenyl unsubstituted or substituted with 1-3 halo groups and pyridyl unsubstituted or substituted with 1-3 halo groups. Within this subset of the invention, all other variables are as originally defined with respect to formula I.

Another subset of compounds that is of interest relates to compounds of formula I or Ia, or a pharmaceutically acceptable salt or solvate thereof, wherein ring B represents a phenyl or thiophene ring and each R⁴ is selected from hydrogen and halo, and in particular, fluoro. Within this subset of the invention, all other variables are as originally defined with respect to formula I.

Another subset of compounds that is of interest relates to compounds of formula I or Ia, or a pharmaceutically acceptable salt or solvate thereof, wherein ring B represents a cyclohexene ring with 1-3 R⁴ groups selected from hydrogen, halo, C₁₋₃alkyl and 0-1 R⁴ groups is selected from heteroaryl and aryl, said C₁₋₃alkyl, heteroaryl and aryl groups optionally substituted with 1-3 halo groups, and 1 OC₁₋₃alkyl, OH or NH₂ group. Within this subset of the invention, all other variables are as originally defined with respect to formula I.

Another subset of compounds that is of interest relates to compounds of formula I or Ia, or a pharmaceutically acceptable salt or solvate thereof, wherein ring B represents a cyclohexene ring, and 3 R⁴ groups are present and represent H or methyl. Within this subset of the invention, all other variables are as originally defined with respect to formula I.

Another subset of compounds that is of interest relates to compounds of formula I or Ia, or a pharmaceutically acceptable salt or solvate thereof, wherein ring B represents a cyclohexene ring, and 3 R⁴ groups are present 1 of which represents phenyl substituted with 1-3 halo atoms, and the remainder of the R⁴ groups represent H. Within this subset of the invention, all other variables are as originally defined with respect to formula I.

Another subset of compounds that is of interest relates to compounds of formula I or Ia, or a pharmaceutically acceptable salt or solvate thereof, wherein each R′ is H or is independently selected from the group consisting of:

(a) halo, OH, CO₂H, CN, NH₂, S(O)₂R^{e}, C(O)R^{e}, OC(O)R^{e} and CO₂R^{e}, wherein R^{e} represents C₁₋₄alkyl or phenyl, said C₁₋₄alkyl and phenyl each being optionally substituted with 1-3 groups, 1-3 of which are selected from halo and C₁₋₃alkyl, and 1-2 of which are selected from the group consisting of: OC₁₋₃alkyl, haloC₁₋₃alkyl, haloC₁₋₃alkoxy, OH, NH₂ and NHC₁₋₃alkyl; and

(b) phenyl or a 5-6 membered heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH; CO₂H; CN; NH₂ and S(O)O₂R^{e} wherein R^{e} is as described above; and
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, NH₂, NHC₄alkyl, N(C₁₋₄alkyl)₂ and CN. Within this subset of the invention, all other variables are as originally defined with respect to formula I.

More particularly, another subset of compounds that is of interest relates to compounds of formula I or Ia, or a pharmaceutically acceptable salt or solvate thereof, wherein each R′ is selected from the group consisting of: H, halo, NH₂ and OH. Within this subset of the invention, all other variables are as originally defined with respect to formula I.

Even more particularly, another subset of compounds that is of interest relates to compounds of formula I or Ia, or a pharmaceutically acceptable salt or solvate thereof, wherein 2 R¹ moieties are H and 1 R¹ moiety is selected from the group consisting of phenyl or a 5-6 membered heteroaryl group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 1 of which is selected from the group consisting of OH, CN and NH₂. Within this subset of the invention, all other variables are as originally defined with respect to formula I.

Another subset of compounds that is of interest relates to compounds of formula I or Ia, or a pharmaceutically acceptable salt or solvate thereof, wherein one R′ group is a member selected from the group consisting of: phenyl and pyridyl substituted with 1-3 of F, Cl, OH, CH₃ and OCH₃, and the remaining R′ groups represent hydrogen. Within this subset of the invention, all other variables are as originally defined with respect to formula I.

Another subset of compounds that is of interest relates to compounds of formula I or Ia, or a pharmaceutically acceptable salt or solvate thereof, wherein 3 R¹ groups are present, one of which represents a pyridyl ring substituted with a fluorine atom, and the remainder of the R¹ groups represent hydrogen. Within this subset of the invention, all other variables are as originally defined with respect to formula I.

Another subset of compounds that is of interest relates to compounds of formula I or Ia, or a pharmaceutically acceptable salt or solvate thereof, wherein 3 Rlgroups are present, one of which represents a pyridyl ring substituted with a hydroxyl group, and the remainder of the Rlgroups represent hydrogen. Within this subset of the invention, all other variables are as originally defined with respect to formula I.

A subset of compounds that is of particular interest relates to compounds of formula I or Ia, or a pharmaceutically acceptable salt or solvate thereof wherein:

ring A represents a 6-10 membered aryl, or a 5-13 membered heteroaryl or a non-aromatic or partially aromatic heterocyclic group, containing at least one heteroatom selected from O, S, and N, and 0-2 additional N atoms;

ring B is selected from phenyl, thiophene and cyclohexenyl;

one of x and y is 0 and the other is 1;

R^{a}, R^{b} and R^{c} are selected from H and CH₃;

R² and R³ represent H;

each R¹ is H or is independently selected from the group consisting of:

(a) halo, OH, CO₂H, CN, NH₂, S(O)₀₋₂R^{e}, C(O)R^{e}, OC(O)R^{e} and CO₂R^{e}, wherein R^{e} represents C₁₋₄alkyl or phenyl, said C₁₋₄alkyl and phenyl each being optionally substituted with 1-3 groups, 1-3 of which are selected from halo and C₁₋₃alkyl, and 1-2 of which are selected from the group consisting of: OC₁₋₃alkyl, haloC₁₋₃alkyl, haloC₁₋₃alkoxy, OH, NH₂ and NHC₁₋₃alkyl; and

(b) phenyl or a 5-6 membered heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH; CO₂H; CN; NH₂ and S(O)₀₋₂R^{e} wherein R^{e} is as described above; and
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, NH₂, NHC₁₋₄alkyl, N(C₁₋₄alkyl)₂ and CN, and when ring B represents phenyl or thiophene, each R⁴ group is selected from hydrogen and halo, and in particular, fluoro, and when ring B represents a cyclohexene ring, 1-3 R⁴ groups are selected from hydrogen, halo and C₁₋₃alkyl and 0-1 R⁴ groups are selected from heteroaryl and aryl, said C₁₋₃alkyl, heteroaryl and aryl groups being optionally substituted with 1-3 halo groups, and 1 OC₁₋₃alkyl, OH or NH₂ group. Within this subset of the invention, all other variables are as originally defined with respect to formula I.

Representative examples of species that are of interest are shown below in Table I Within this subset of compounds, all other variables are as originally defined with respect to formula I.

TABLE I

| | |
|---|---|
| | COMPOUND 1 |
| 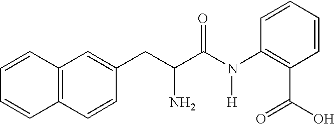 | |
| | COMPOUND 2 |
| 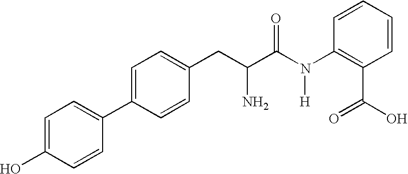 | |
| | COMPOUND 3 |
| 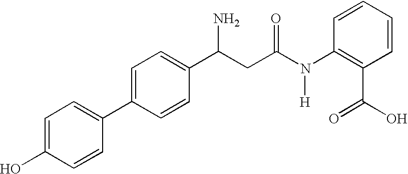 | |
| | COMPOUND 4 |
| 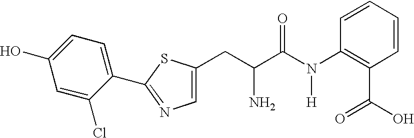 | |
| | COMPOUND 5 |
| 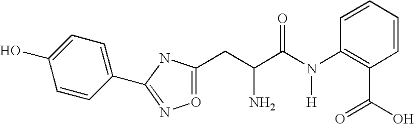 | |
| | COMPOUND 6 |
| 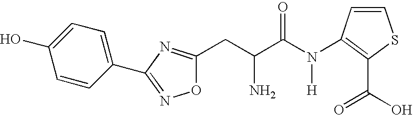 | |
| | COMPOUND 7 |
| 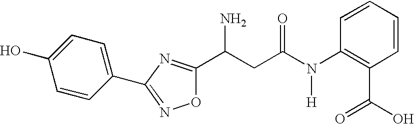 | |
| | COMPOUND 8 |
| 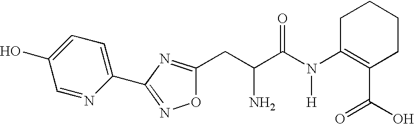 | |
| | COMPOUND 9 |
| 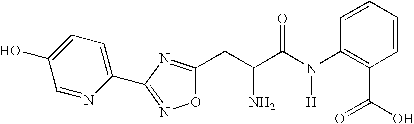 | |
| | COMPOUND 10 |
| 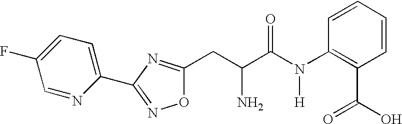 | |
| | COMPOUND 11 |
| 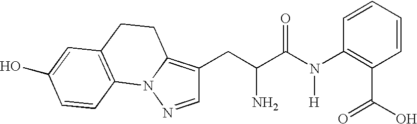 | |
| | COMPOUND 12 |
| 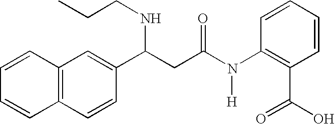 | |
| | COMPOUND 13 |
| 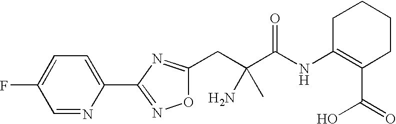 | |
| | COMPOUND 14 |
| 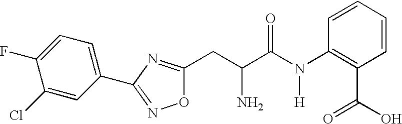 | |
| | COMPOUND 15 |
| 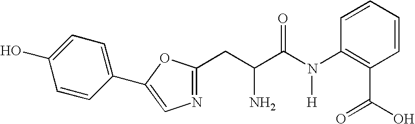 | |
| | COMPOUND 16 |
| 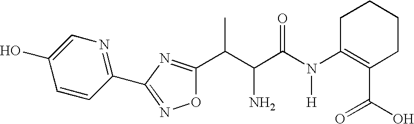 | |
| | COMPOUND 17 |
| 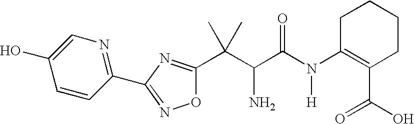 | |
| | COMPOUND 18 |
| 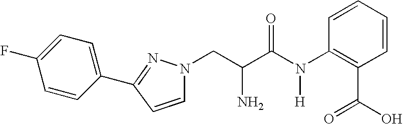 | |
| | COMPOUND 19 |
| 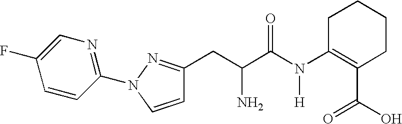 | |
| | COMPOUND 20 |
| 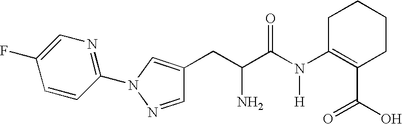 | |
| | COMPOUND 21 |
| 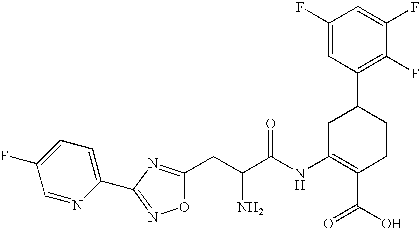 | |
| | COMPOUND 22 |
| 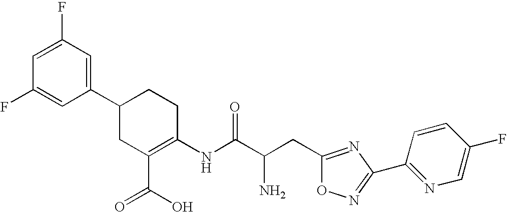 | |
| | COMPOUND 23 |
| 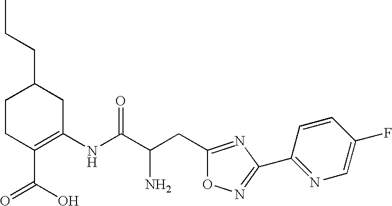 | |
| | COMPOUND 24 |
| 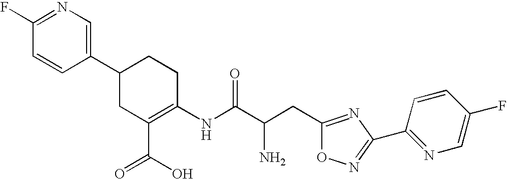 | |
| | COMPOUND 25 |
| 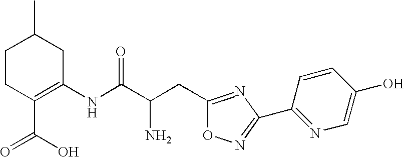 | |
| | COMPOUND 26 |
| 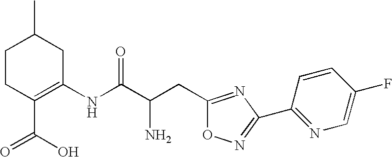 | |
| | COMPOUND 27 |
| 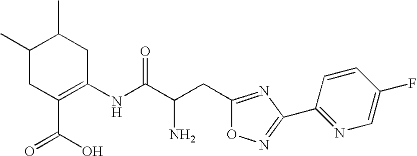 | |
| | COMPOUND 28 |
| 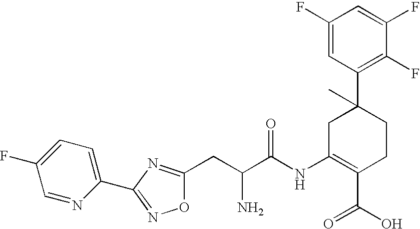 | |
| | COMPOUND 29 |
| 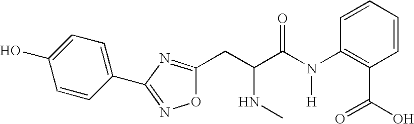 | |
| | COMPOUND 30 |
| 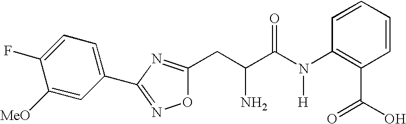 | |
| | COMPOUND 31 |
| 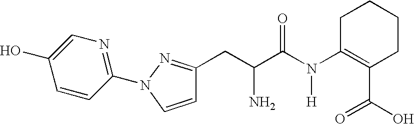 | |
| | COMPOUND 32 |
| 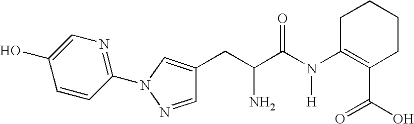 | |
| | COMPOUND 33 |
| 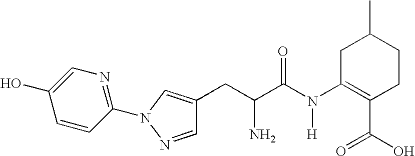 | |

Pharmaceutically acceptable salts and solvates thereof are included as well.

All of the compounds of formula I contain asymmetric centers and can thus occur as racemates and racemic mixtures, single enantiomers, diastereomeric mixtures and individual diastereomers. All such isomeric forms are included.

Moreover, chiral compounds possessing one stereocenter of general formula I or Ia, may be resolved into their enantiomers in the presence of a chiral environment using methods known to those skilled in the art. Chiral compounds possessing more than one stereocenter may be separated into their diastereomers in an achiral environment on the basis of their physical properties using methods known to those skilled in the art. Single diastereomers that are obtained in racemic form may be resolved into their enantiomers as described above.

If desired, racemic mixtures of compounds may be separated so that individual enantiomers are isolated. The separation can be carried out by methods well known in the art, such as the coupling of a racemic mixture of compounds of Formula I or Ia, to an enantiomerically pure compound to form a diastereomeric mixture, which is then separated into individual diastereomers by standard methods, such as fractional crystallization or chromatography. The coupling reaction is often the formation of salts using an enantiomerically pure acid or base. The diasteromeric derivatives may then be converted to substantially pure enantiomers by cleaving the added chiral residue from the diastereomeric compound.

The racemic mixture of the compounds of Formula I or Ia can also be separated directly by chromatographic methods utilizing chiral stationary phases, which methods are well known in the art.

Alternatively, enantiomers of compounds of the general Formula I may be obtained by stereoselective synthesis using optically pure starting materials or reagents. Some of these optically pure starting materials may be obtained cormnercially from the chiral pool, such as natural amino acids.

Some of the compounds described herein exist as tautomers, which have different points of attachment for hydrogen accompanied by one or more double bond shifts. For example, a ketone and its enol form are keto-enol tautomers. Or for example, a 2-hydroxyquinoline can reside in the tautomeric 2-quinolone form. The individual tautomers as well as mixtures thereof are included.

### Dosing Information

The dosages of compounds of formula I or a pharmaceutically acceptable salt or solvate thereof vary within wide limits. The specific dosage_regimen and levels for any particular patient will depend upon a variety of factors including the age, body weight, general health, sex, diet, time of administration, route of administration, rate of excretion, drug combination and the severity of the patient's condition. Consideration of these factors is well within the purview of the ordinarily skilled clinician for the purpose of determining the therapeutically effective or prophylactically effective dosage amount needed to prevent, counter, or arrest the progress of the condition. Generally, the compounds will be administered in amounts ranging from as low as about 0.01 mg/day to as high as about 2000 mg/day, in single or divided doses. A representative dosage is about 0.1 mg/day to about 1 g/day. Lower dosages can be used initially, and dosages increased to further minimize any untoward effects. It is expected that the compounds described herein will be administered on a daily basis for a length of time appropriate to treat or prevent the medical condition relevant to the patient, including a course of therapy lasting months, years or the life of the patient.

### Combination Therapy

One or more additional active agents may be administered with the compounds described herein. The additional active agent or agents can be lipid modifying compounds or agents having other pharmaceutical activities, or agents that have both lipid-modifying effects and other pharmaceutical activities. Examples of additional active agents which may be employed include but are not limited to HMG-CoA reductase inhibitors, which include statins in their lactonized or dihydroxy open acid forms and pharmaceutically acceptable salts and esters thereof, including but not limited to lovastatin (see U.S. Pat. No. 4,342,767), simvastatin (see U.S. Pat. No. 4,444,784), dihydroxy open-acid simvastatin, particularly the ammonium or calcium salts thereof, pravastatin, particularly the sodium salt thereof (see U.S. Pat. No. 4,346,227), fluvastatin particularly the sodium salt thereof (see U.S. Pat. No. 5,354,772), atorvastatin, particularly the calcium salt thereof (see U.S. Pat. No. 5,273,995), pitavastatin also referred to as NK-104 (see PCT international publication number WO 97/23200) and rosuvastatin, also known as CRESTOR®; see U.S. Pat. No. 5,260,440); HMG-CoA synthase inhibitors; squalene epoxidase inhibitors; squalene synthetase inhibitors (also known as squalene synthase inhibitors), acyl-coenzyme A: cholesterol acyltransferase (ACAT) inhibitors including selective inhibitors of ACAT-1 or ACAT-2 as well as dual inhibitors of ACAT-1 and -2; microsomal triglyceride transfer protein (MTP) inhibitors; endothelial lipase inhibitors; bile acid sequestrants; LDL receptor inducers; platelet aggregation inhibitors, for example glycoprotein IIb/IIIa fibrinogen receptor antagonists and aspirin; human peroxisome proliferator activated receptor gamma (PPAR-gamma) agonists including the compounds commonly referred to as glitazones for example pioglitazone and rosiglitazone and, including those compounds included within the structural class known as thiazolidine diones as well as those PPAR-gamma agonists outside the thiazolidine dione structural class; PPAR-alpha agonists such as clofibrate, fenofibrate including micronized fenofibrate, and gemfibrozil; PPAR dual alpha/gamma agonists; vitamin B₆ (also known as pyridoxine) and the pharmaceutically acceptable salts thereof such as the HCl salt; vitamin B₁₂ (also known as cyanocobalamin); folic acid or a pharmaceutically acceptable salt or ester thereof such as the sodium salt and the methylglucamine salt; anti-oxidant vitamins such as vitamin C and E and beta carotene; beta-blockers; angiotensin II antagonists such as losartan; angiotensin converting enzyme inhibitors such as enalapril and captopril; renin inhibitors, calcium channel blockers such as nifedipine and diltiazem; endothelin antagonists; agents that enhance ABCA1 gene expression; cholesteryl ester transfer protein (CETP) inhibiting compounds, 5-lipoxygenase activating protein (FLAP) inhibiting compounds, 5-lipoxygenase (5-LO) inhibiting compounds, framesoid X receptor (FXR) ligands including both antagonists and agonists; Liver X Receptor (LXR)-alpha ligands, LXR-beta ligands, bisphosphonate compounds such as alendronate sodium; cyclooxygenase-2 inhibitors such as rofecoxib and celecoxib; and compounds that attenuate vascular inflammation.

Cholesterol absorption inhibitors can also be used in the present invention. Such compounds block the movement of cholesterol from the intestinal lumen into enterocytes of the small intestinal wall, thus reducing serum cholesterol levels. Examples of cholesterol absorption inhibitors are described in U.S. Pat. Nos. 5,846,966, 5,631,365, 5,767,115, 6,133,001, 5,886,171, 5,856,473, 5,756,470, 5,739,321, 5,919,672, and in PCT application Nos. WO 00/63703, WO 00/60107, WO 00/38725, WO 00/34240, WO 00/20623, WO 97/45406, WO 97/16424, WO 97/16455, and WO 95/08532. The most notable cholesterol absorption inhibitor is ezetimibe, also known as 1-(4-fluorophenyl)-3(R)-[3(S)-(4-fluorophenyl)-3-hydroxypropyl)]-4(S)-(4-hydroxyphenyl)-2-azetidinone, described in U.S. Pat. Nos. 5,767,115 and 5,846,966.

Therapeutically effective amounts of cholesterol absorption inhibitors include dosages of from about 0.01 mg/kg to about 30 mg/kg of body weight per day, preferably about 0.1 mg/kg to about 15 mg/kg.

For diabetic patients, the compounds used in the present invention can be administered with conventional diabetic medications. For example, a diabetic patient receiving treatment as described herein may also be taking insulin or an oral antidiabetic medication. One example of an oral antidiabetic medication useful herein is metformin.

In the event that these niacin receptor agonists induce some degree of vasodilation, it is understood that the compounds of formula I may be co-dosed with a vasodilation suppressing agent. Consequently, one aspect of the methods described herein relates to the use of a compound of formula I or a pharmaceutically acceptable salt or solvate thereof in combination with a compound that reduces flushing. Conventional compounds such as aspirin, ibuprofen, naproxen, indomethacin, other NSAIDs, COX-2 selective inhibitors and the like are useful in this regard, at conventional doses. Alternatively, DP antagonists are useful as well. Doses of the DP receptor antagonist and selectivity are such that the DP antagonist selectively modulates the DP receptor without substantially modulating the CRTH2 receptor. In particular, the DP receptor antagonist ideally has an affinity at the DP receptor (i.e., Kᵢ) that is at least about 10 times higher (a numerically lower K; value) than the affinity at the CRTH2 receptor. Any compound that selectively interacts with DP according to these guidelines is deemed “Dselective”. This is in accordance with US Published Application No. 2004/0229844A1 published on Nov. 18, 2004.

Dosages for DP antagonists as described herein, that are useful for reducing or preventing the flushing effect in mammalian patients, particularly humans, include dosages ranging from as low as about 0.01 mg/day to as high as about 100 mg/day, administered in single or divided daily doses. Preferably the dosages are from about 0.1 mg/day to as high as about 1.0 g/day, in single or divided daily doses.

Examples of compounds that are particularly useful for selectively antagonizing DP receptors and suppressing the flushing effect include the compounds that are disclosed in WO2004/103370A1 published on Dec. 2, 2004, as well as the pharmaceutically acceptable salts and solvates thereof.

The compound of formula I or a pharmaceutically acceptable salt or solvate thereof and the DP antagonist can be administered together or sequentially in single or multiple daily doses, e.g., bid, tid or qid, without departing from the invention. If sustained release is desired, such as a sustained release product showing a release profile that extends beyond 24 hours, dosages may be administered every other day. However, single daily doses are preferred. Likewise, morning or evening dosages can be utilized.

### Salts and Solvates

Salts and solvates of the compounds of formula I are also included in the present invention, and numerous pharmaceutically acceptable salts and solvates of nicotinic acid are useful in this regard. Alkali metal salts, in particular, sodium and potassium, form salts that are useful as described herein. Likewise alkaline earth metals, in particular, calcium and magnesium, form salts that are useful as described herein. Various salts of amines, such as ammonium and substituted ammonium compounds also form salts that are useful as described herein. Similarly, solvated forms of the compounds of formula I are useful within the present invention. Examples include the hemihydrate, mono-, di-, tri- and sesquihydrate.

The compounds of the invention also include esters that are pharmaceutically acceptable, as well as those that are metabolically labile. Metabolically labile esters include C₁₋₄ alkyl esters, preferably the ethyl ester. Many prodrug strategies are known to those skilled in the art. One such strategy involves engineered amino acid anhydrides possessing pendant nucleophiles, such as lysine, which can cyclize upon themselves, liberating the free acid. Similarly, acetone-ketal diesters, which can break down to acetone, an acid and the active acid, can be used.

Zwitterionic forms of the compounds of formula I are included.

The compounds used in the present invention can be administered via any conventional route of administration. The preferred route of administration is oral.

### Pharmaceutical Compositions

The pharmaceutical compositions described herein are generally comprised of a compound of formula I or a pharmaceutically acceptable salt or solvate thereof, in combination with a pharmaceutically acceptable carrier.

Examples of suitable oral compositions include tablets, capsules, troches, lozenges, suspensions, dispersible powders or granules, emulsions, syrups and elixirs. Examples of carrier ingredients include diluents, binders, disintegrants, lubricants, sweeteners, flavors, colorants, preservatives, and the like. Examples of diluents include, for example, calcium carbonate, sodium carbonate, lactose, calcium phosphate and sodium phosphate. Examples of granulating and disintegrants include corn starch and alginic acid. Examples of binding agents include starch, gelatin and acacia. Examples of lubricants include magnesium stearate, calcium stearate, stearic acid and talc. The tablets may be uncoated or coated by known techniques. Such coatings may delay disintegration and thus, absorption in the gastrointestinal tract and thereby provide a sustained action over a longer period.

In one embodiment of the invention, about 1 mg to about 1000 mg of a compound of formula I, or a pharmaceutically acceptable solvate or solvate thereof, is combined with a pharmaceutically acceptable carrier to form a pharmaceutical composition. Preferably this is a tablet or a capsule.

In another embodiment of the invention, a compound of formula I or a pharmaceutically acceptable salt or solvate thereof is combined with another therapeutic agent and the carrier to form a fixed combination product. This fixed combination product is preferably a tablet or capsule for oral use.

More particularly, in another embodiment of the invention, a compound of formula I or a pharmaceutically acceptable salt or solvate thereof (about 1 to about 1000 mg) and the second therapeutic agent (about 1 to about 500 mg) are combined with the pharmaceutically acceptable carrier, providing a tablet or capsule for oral use.

Sustained release over a longer period of time may be particularly important in the formulation. A time delay material such as glyceryl monostearate or glyceryl distearate may be employed. The dosage form may also be coated by the techniques described in the U.S. Pat. Nos. 4,256,108; 4,166,452 and 4,265,874 to form osmotic therapeutic tablets for controlled release.

Other controlled release technologies are also available and are included herein. Typical ingredients that are useful to slow the release of nicotinic acid in sustained release tablets include various cellulosic compounds, such as methylcellulose, ethylcellulose, propylcellulose, hydroxypropylcellulose, hydroxyethylcellulose, hydroxypropylmethylcellulose, microcrystalline cellulose, starch and the like. Various natural and synthetic materials are also of use in sustained release formulations. Examples include alginic acid and various alginates, polyvinyl pyrrolidone, tragacanth, locust bean gum, guar gum, gelatin, various long chain alcohols, such as cetyl alcohol and beeswax.

Optionally and of even more interest is a tablet as described above, comprised of a compound of formula I or a pharmaceutically acceptable salt or solvate thereof, and further containing an HMG Co-A reductase inhibitor, such as simvastatin or atorvastatin. This particular embodiment optionally contains the DP antagonist as well.

Typical release time frames for sustained release tablets in accordance with the present invention range from about 1 to as long as about 48 hours, preferably about 4 to about 24 hours, and more preferably about 8 to about 16 hours.

Hard gelatin capsules constitute another solid dosage form for oral use. Such capsules similarly include the active ingredients mixed with carrier materials as described above. Soft gelatin capsules include the active ingredients mixed with water-miscible solvents such as propylene glycol, PEG and ethanol, or an oil such as peanut oil, liquid paraffin or olive oil.

Aqueous suspensions are also contemplated as containing the active material in admixture with excipients suitable for the manufacture of aqueous suspensions. Such excipients include suspending agents, for example sodium carboxymethylcellulose, methylcellulose, hydroxypropylmethylcellulose, sodium alginate, polyvinylpyrrolidone, tragacanth and acacia; dispersing or wetting agents, e.g., lecithin; preservatives, e.g., ethyl, or n-propyl para-hydroxybenzoate, colorants, flavors, sweeteners and the like.

Dispersible powders and granules suitable for preparation of an aqueous suspension by the addition of water provide the active ingredients in admixture with a dispersing or wetting agent, suspending agent and one or more preservatives. Suitable dispersing or wetting agents and suspending agents are exemplified by those already mentioned above.

Syrups and elixirs may also be formulated.

More particularly, a pharmaceutical composition that is of interest is a sustained release tablet that is comprised of a compound of formula I or a pharmaceutically acceptable salt or solvate thereof, and a DP receptor antagonist that is selected from the group consisting of compounds A through AJ in combination with a pharmaceutically acceptable carrier.

Yet another pharmaceutical composition that is of more interest is comprised of a compound of formula I or a pharmaceutically acceptable salt or solvate thereof and a DP antagonist compound selected from the group consisting of compounds A, B, D, E, X, AA, AF, AG, AH, AI and AJ, in combination with a pharmaceutically acceptable carrier.

Yet another pharmaceutical composition that is of more particular interest relates to a sustained release tablet that is comprised of a compound of formula I or a pharmaceutically acceptable salt or solvate thereof, a DP receptor antagonist selected from the group consisting of compounds A, B, D, E, X, AA, AF, AG, AH, AI and AJ, and simvastatin or atorvastatin in combination with a pharmaceutically acceptable carrier.

The term “composition”, in addition to encompassing the pharmaceutical compositions described above, also encompasses any product which results, directly or indirectly, from the combination, complexation or aggregation of any two or more of the ingredients, active or excipient, or from dissociation of one or more of the ingredients, or from other types of reactions or interactions of one or more of the ingredients. Accordingly, the pharmaceutical composition of the present invention encompasses any composition made by admixing or otherwise combining the compounds, any additional active ingredient(s), and the pharmaceutically acceptable excipients.

Another aspect of the invention relates to the use of a compound of formula I or a pharmaceutically acceptable salt or solvate thereof and a DP antagonist in the manufacture of a medicament. This medicament has the uses described herein.

More particularly, another aspect of the invention relates to the use of a compound of formula I or a pharmaceutically acceptable salt or solvate thereof, a DP antagonist and an HMG Co-A reductase inhibitor, such as simvastatin, in the manufacture of a medicament. This medicament has the uses described herein.

Compounds of the present invention have anti-hyperlipidemic activity, causing reductions in LDL-C, triglycerides, apolipoprotein a and total cholesterol, and increases in HDL-C. Consequently, the compounds of the present invention are useful in treating dyslipidemias. The present invention thus relates to the treatment, prevention or reversal of atherosclerosis and the other diseases and conditions described herein, by administering a compound of formula I or a pharmaceutically acceptable salt or solvate in an amount that is effective for treating, preventing or reversing said condition. This is achieved in humans by administering a compound of formula I or a pharmaceutically acceptable salt or solvate thereof in an amount that is effective to treat or prevent said condition, while preventing, reducing or minimizing flushing effects in terms of frequency and/or severity.

One aspect of the invention that is of interest is a method of treating atherosclerosis in a human patient in need of such treatment comprising administering to the patient a compound of formula I or a pharmaceutically acceptable salt or solvate thereof in an amount that is effective for treating atherosclerosis in the absence of substantial flushing.

Another aspect of the invention that is of interest relates to a method of raising serum HDL levels in a human patient in need of such treatment, comprising administering to the patient a compound of formula I or a pharmaceutically acceptable salt or solvate thereof in an amount that is effective for raising serum HDL levels.

Another aspect of the invention that is of interest relates to a method of treating dyslipidemia in a human patient in need of such treatment comprising administering to the patient a compound of formula I or a pharmaceutically acceptable salt or solvate thereof in an amount that is effective for treating dyslipidemia.

Another aspect of the invention that is of interest relates to a method of reducing serum VLDL or LDL levels in a human patient in need of such treatment, comprising administering to the patient a compound of formula I or a pharmaceutically acceptable salt or solvate thereof in an amount that is effective for reducing serum VLDL or LDL levels in the patient in the absence of substantial flushing.

Another aspect of the invention that is of interest relates to a method of reducing serum triglyceride levels in a human patient in need of such treatment, comprising administering to the patient a compound of formula I or a pharmaceutically acceptable salt or solvate thereof in an amount that is effective for reducing serum triglyceride levels.

Another aspect of the invention that is of interest relates to a method of reducing serum Lp(a) levels in a human patient in need of such treatment, comprising administering to the patient a compound of formula I or a pharmaceutically acceptable salt or solvate thereof in an amount that is effective for reducing serum Lp(a) levels. As used herein Lp(a) refers to lipoprotein (a).

Another aspect of the invention that is of interest relates to a method of treating diabetes, and in particular, type 2 diabetes, in a human patient in need of such treatment comprising administering to the patient a compound of formula I or a pharmaceutically acceptable salt or solvate thereof in an amount that is effective for treating diabetes.

Another aspect of the invention that is of interest relates to a method of treating metabolic syndrome in a human patient in need of such treatment comprising administering to the patient a compound of formula I or a pharmaceutically acceptable salt or solvate thereof in an amount that is effective for treating metabolic syndrome.

Another aspect of the invention that is of particular interest relates to a method of treating atherosclerosis, dyslipidemias, diabetes, metabolic syndrome or a related condition in a human patient in need of such treatment, comprising administering to the patient a compound of formula I or a pharmaceutically acceptable salt or solvate thereof and a DP receptor antagonist, said combination being administered in an amount that is effective to treat atherosclerosis, dyslipidemia, diabetes or a related condition in the absence of substantial flushing.

Another aspect of the invention that is of particular interest relates to the methods described above wherein the DP receptor antagonist is selected from the group consisting of compounds A through AJ and the pharmaceutically acceptable salts and solvates thereof.

### Methods of Synthesis for Compounds of Formula I

Representative compounds of formula I have been prepared by the following reaction schemes. It is understood that other synthetic approaches to these structure classes are conceivable to one skilled in the art. Therefore these reaction schemes should not be construed as limiting the scope of the invention. All substituents are as defined above unless indicated otherwise.

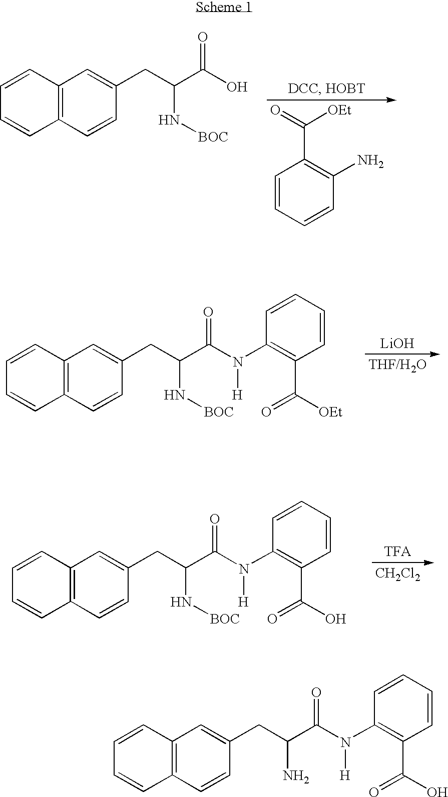

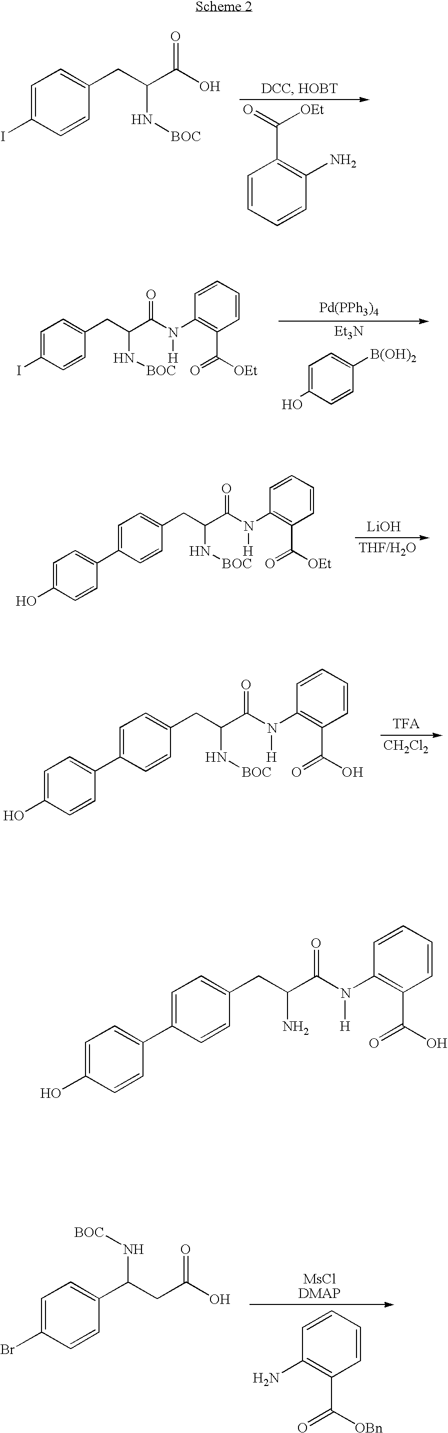

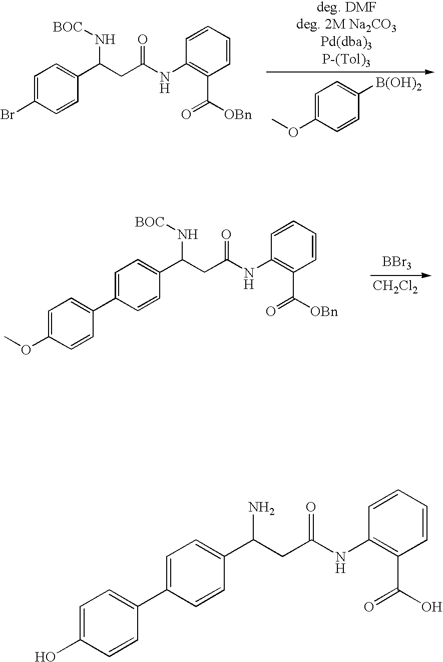

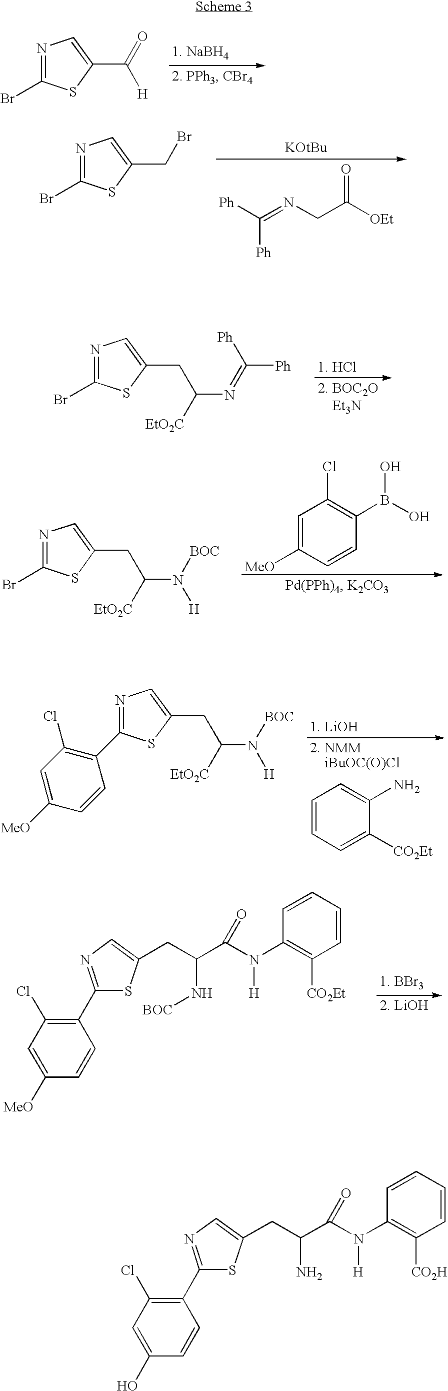

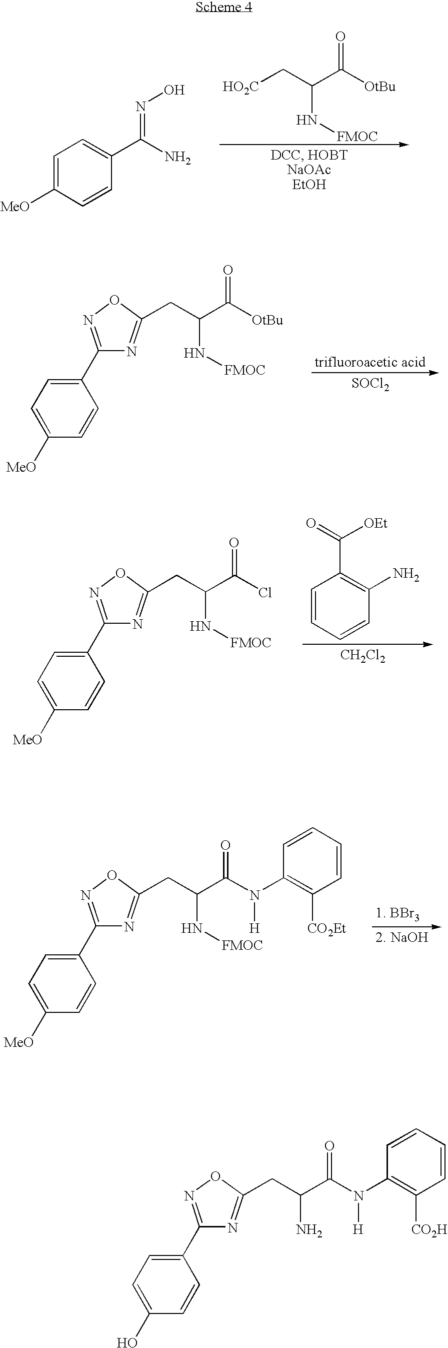

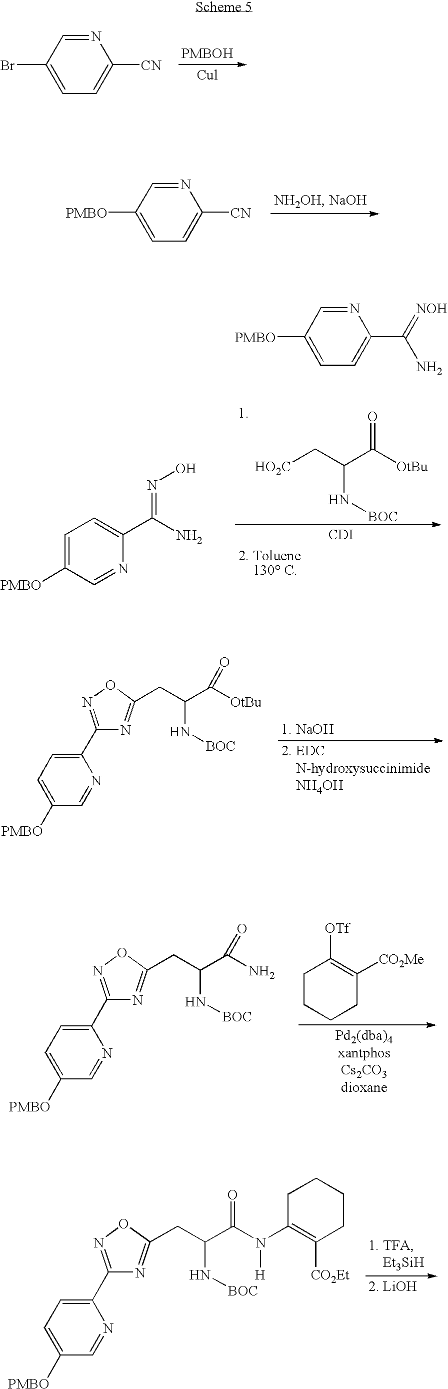

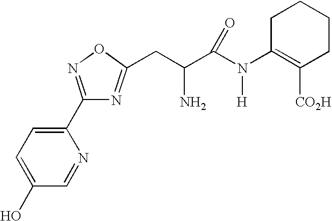

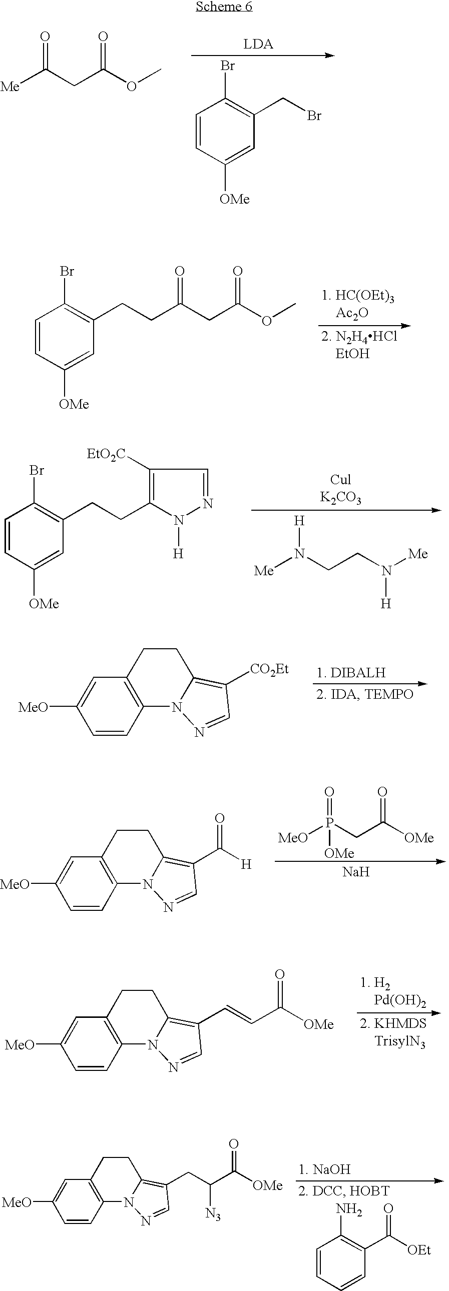

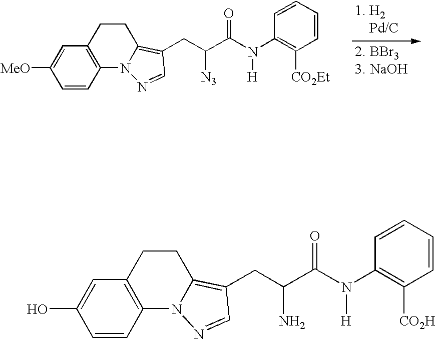

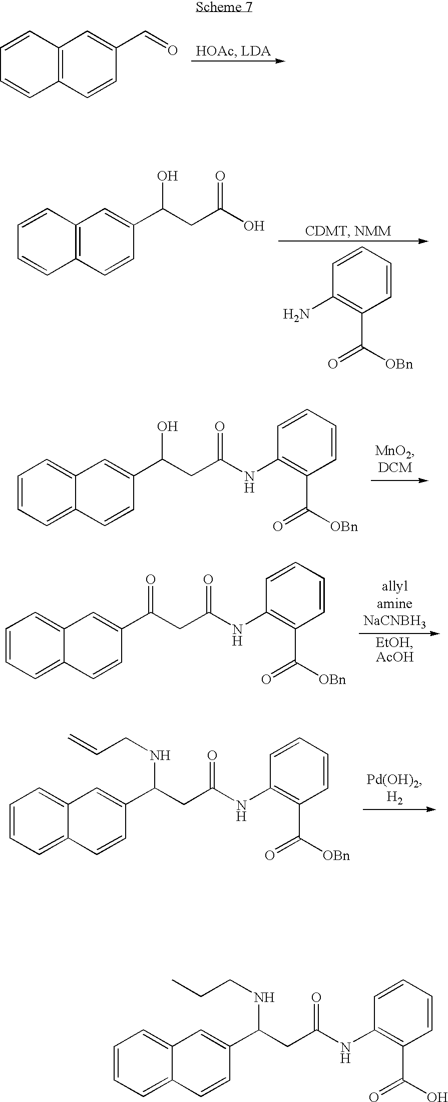

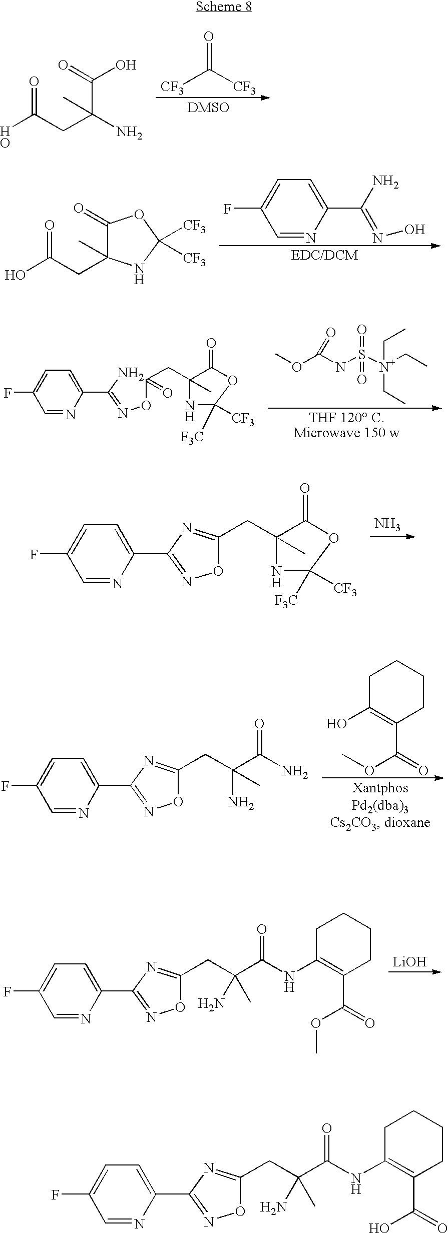

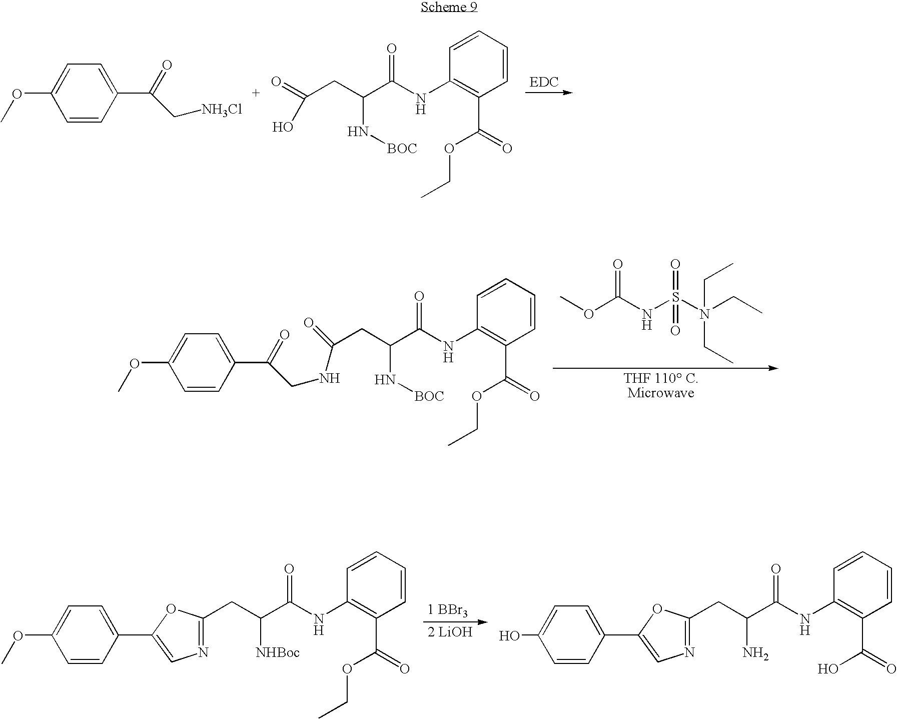

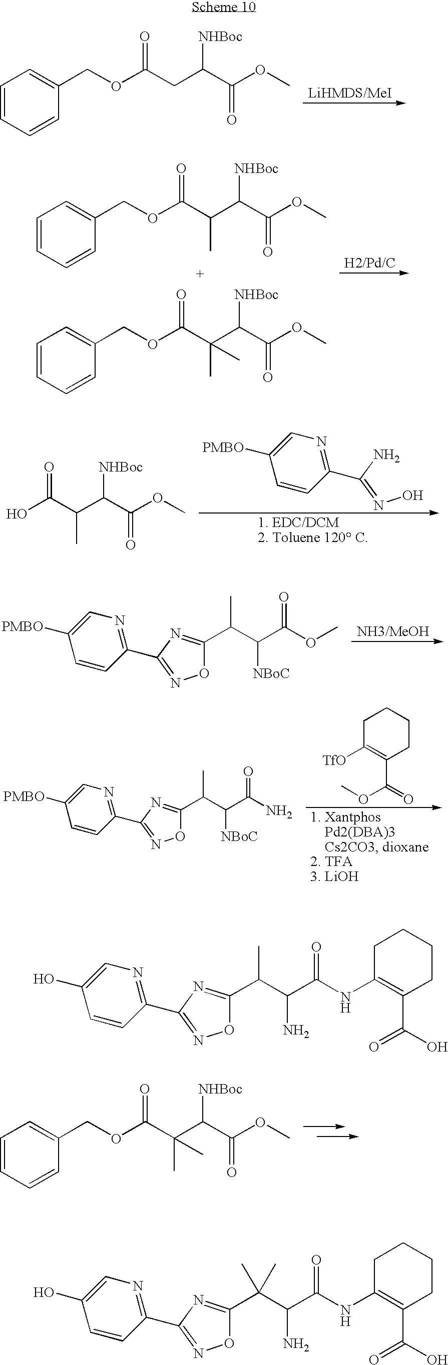

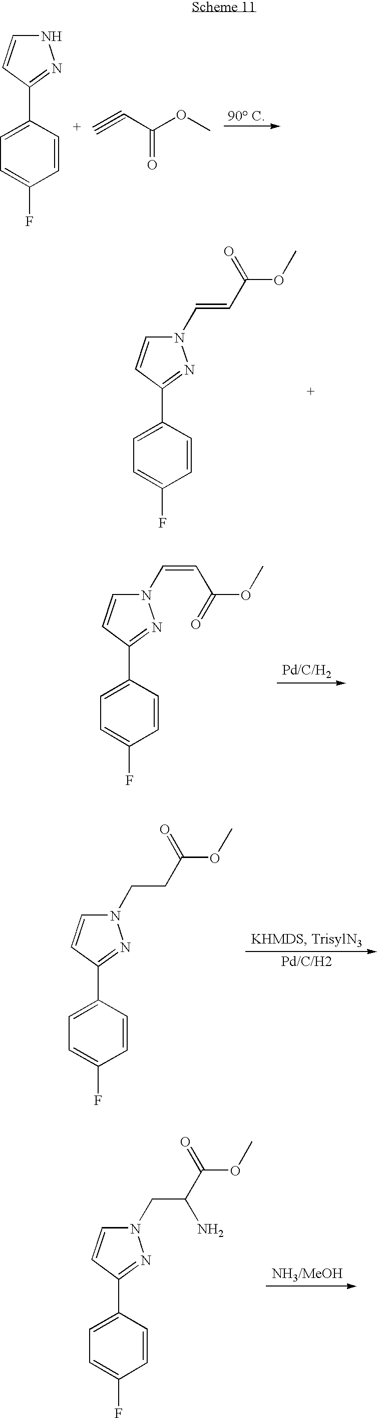

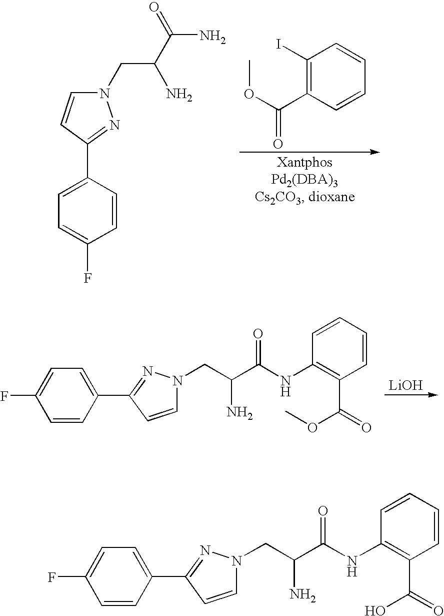

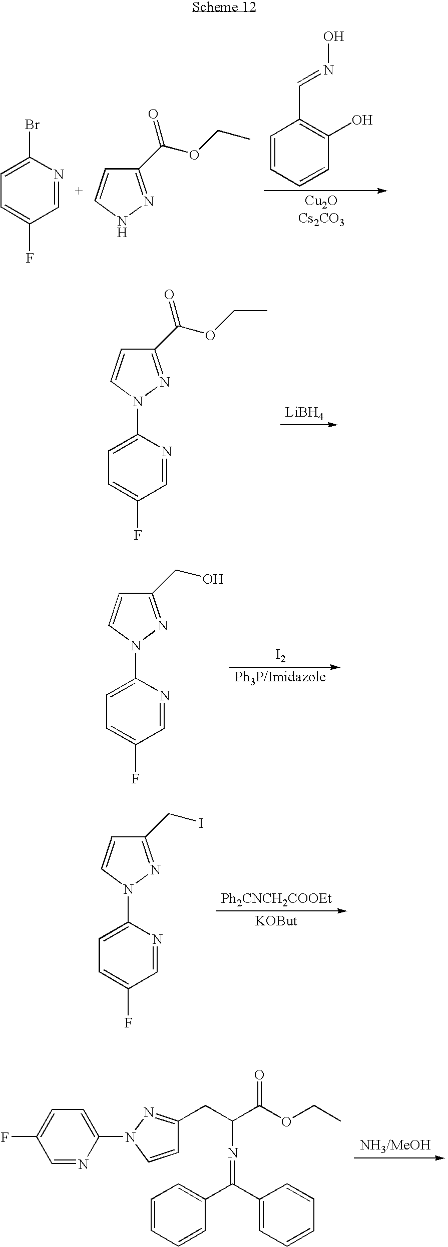

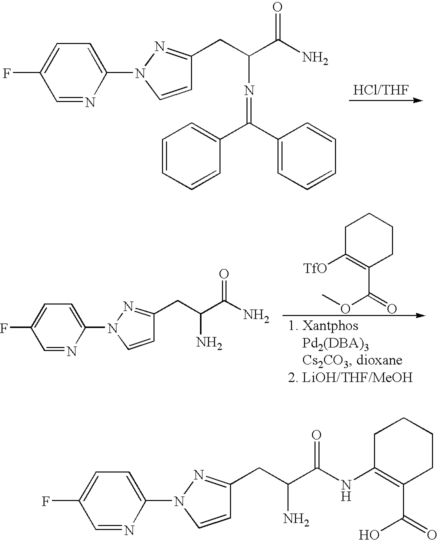

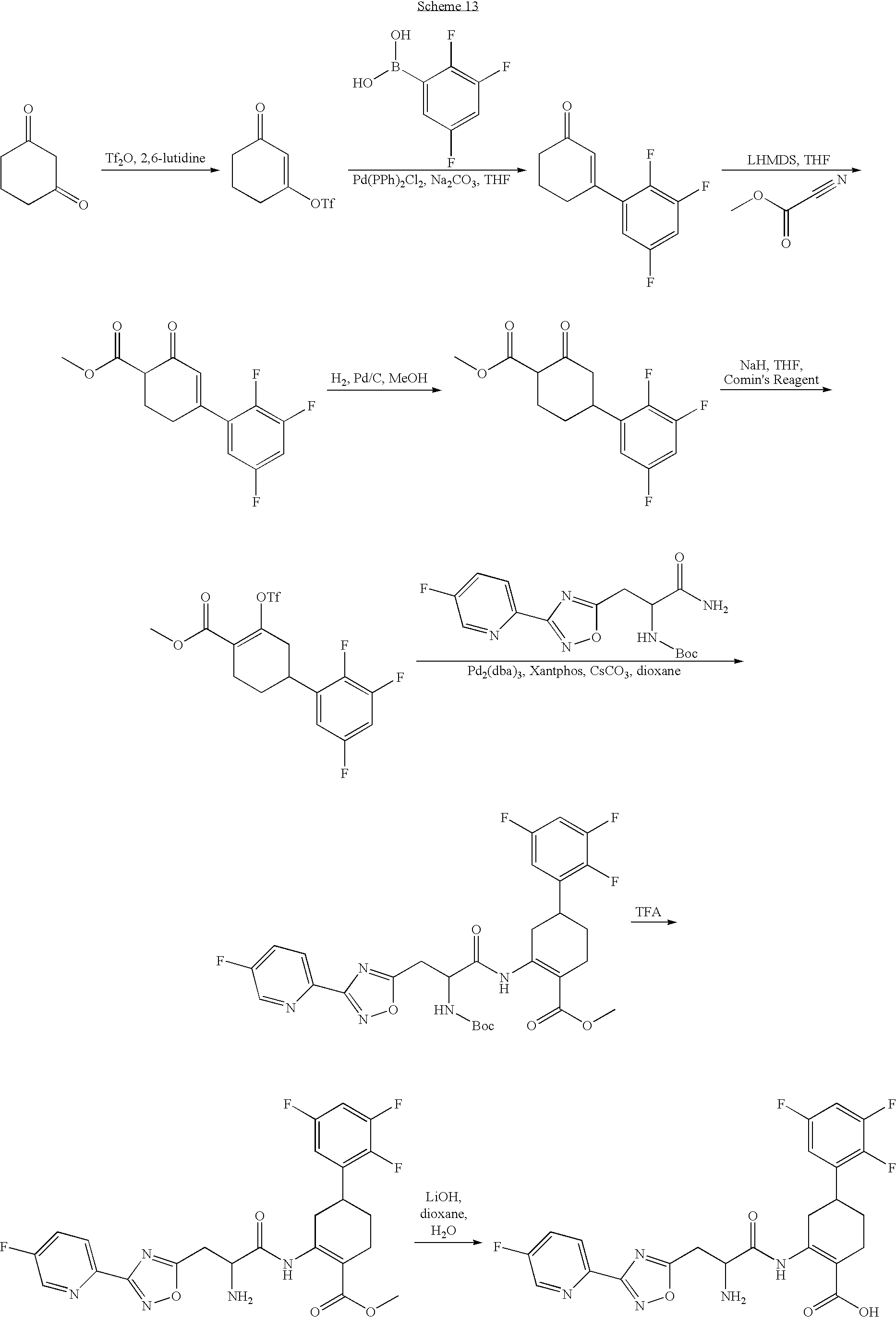

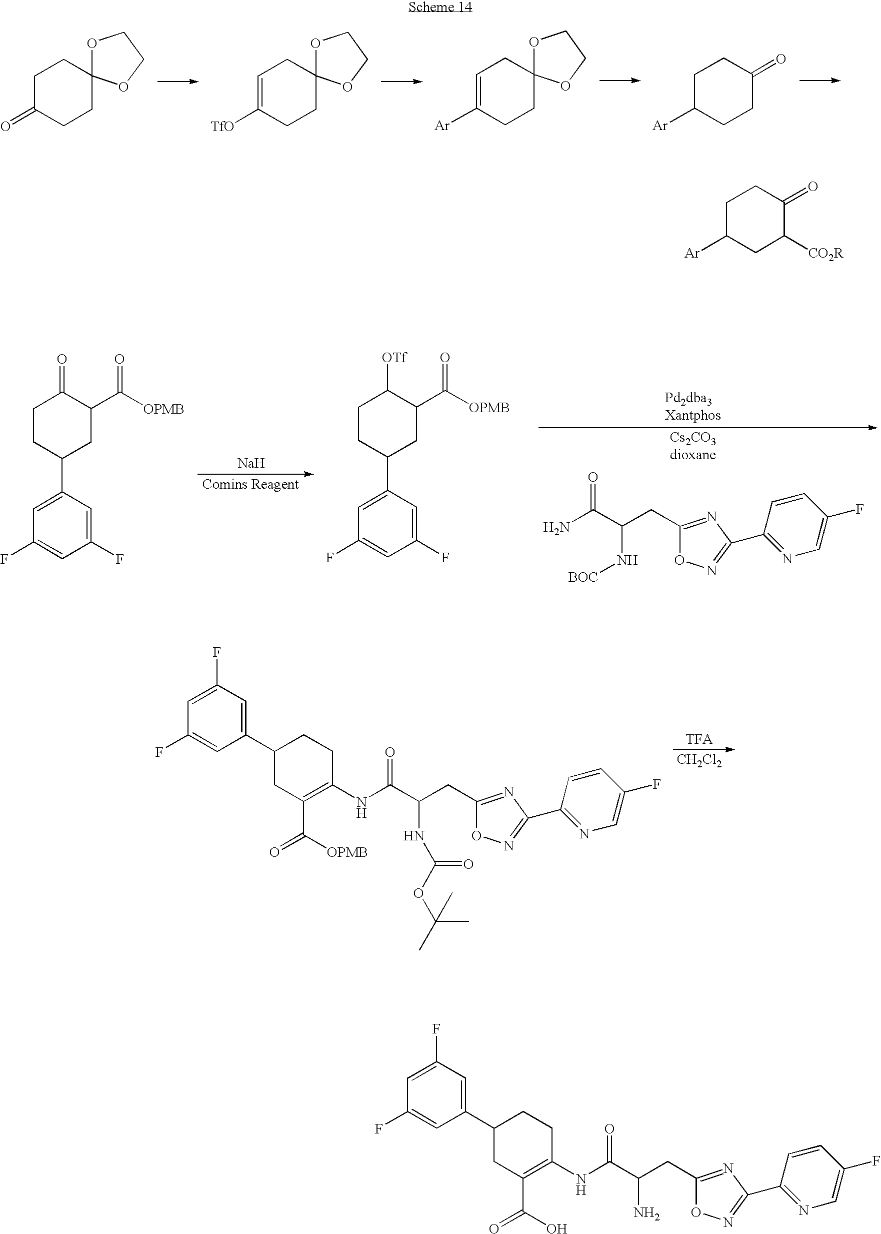

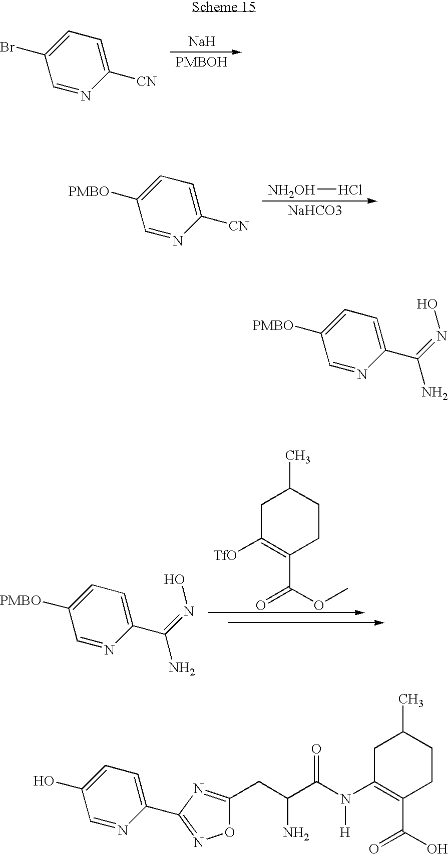

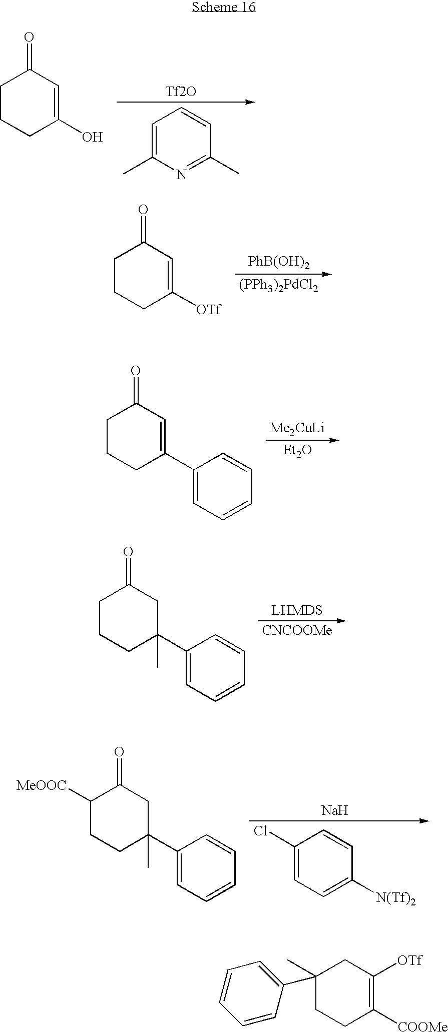

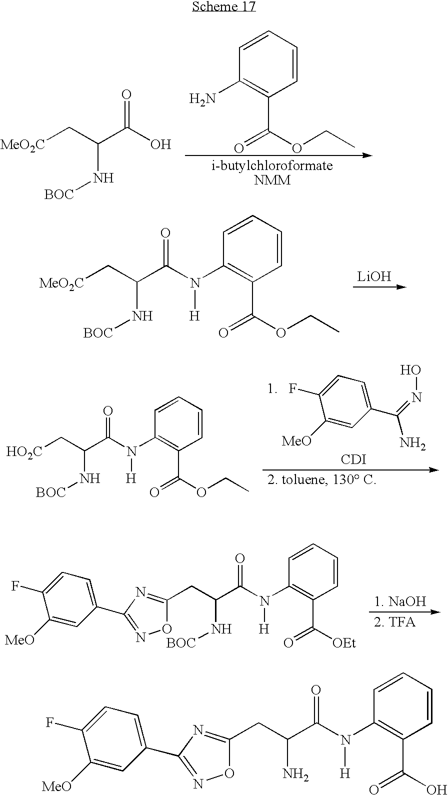

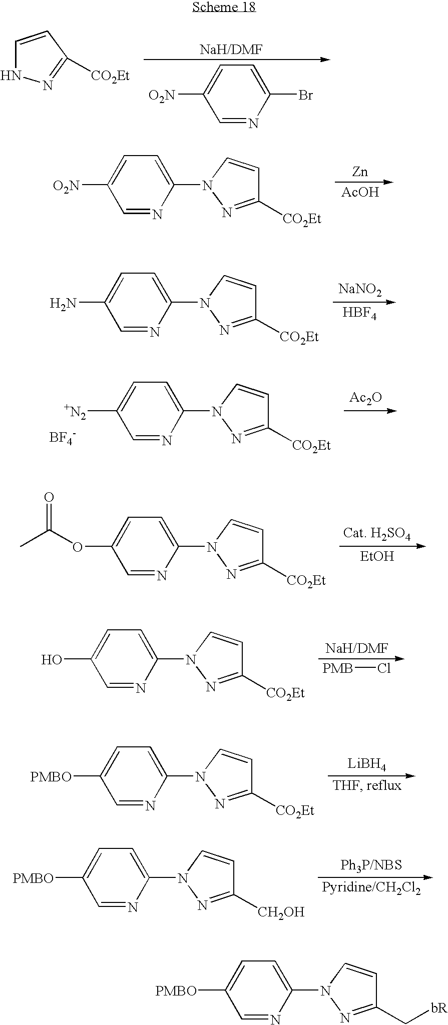

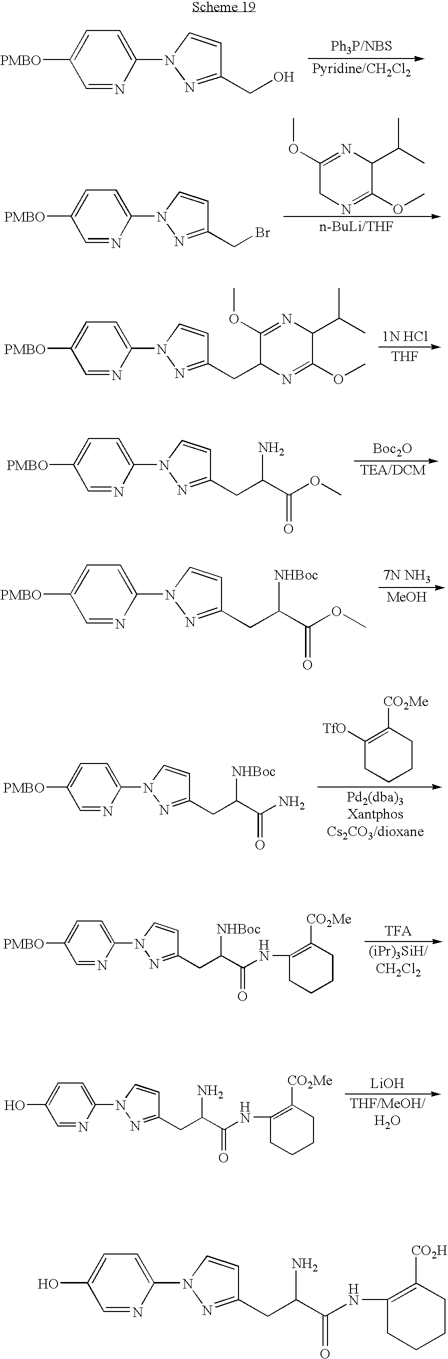

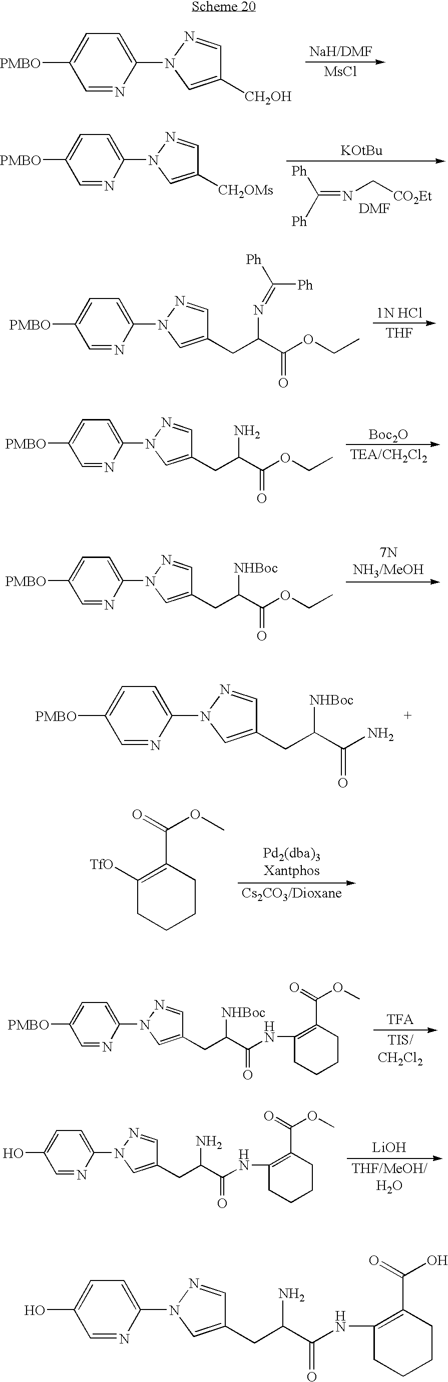

The various organic group transformations and protecting groups utilized herein can be performed by a number of procedures other than those shown in the schemes above. References for other synthetic procedures that can be utililized for the preparation of intermediates or compounds disclosed herein can be found in, for example, M. B. Smith, J. March Advanced Organic Chemistry, 5^{th} Edition, Wiley-Interscience (2001); R. C. Larock Comprehensive Organic Transformations, A Guide to Functional Group Preparations, 2^{nd} Edition, VCH Publishers, Inc. (1999); T. L. Gilchrist Heterocyclic Chemistry, 3^{rd} Edition, Addison Wesley Longman Ltd. (1997); J. A. Joule, K. Mills, G. F. Smith Heterocyclic Chemistry, 3^{rd} Edition, Stanley Thornes Ltd. (1998); G. R. Newkome, W. W. Paudler Contempory Heterocyclic Chemistry, John Wiley and Sons (1982); or Wuts, P. G. M.; Greene, T. W.; Protective Groups in Organic Synthesis, 3^{rd} Edition, John Wiley and Sons, (1999).

### REPRESENTATIVE EXAMPLES

The following examples are provided to more fully illustrate the present invention, and shall not be construed as limiting the scope in any manner. Unless stated otherwise:

(i) all operations were carried out at room or ambient temperature (RT or rt), that is, at a temperature in the range 18-25° C.;

(ii) evaporation of solvent was carried out using a rotary evaporator under reduced pressure (4.5-30 mmHg) with a bath temperature of up to 50° C.;

(iii) the course of reactions was followed by thin layer chromatography (TLC) and/or tandem high performance liquid chromatography (HPLC) followed by mass spectroscopy (MS), herein termed LCMS, and any reaction times are given for illustration only;

(iv) yields, if given, are for illustration only;

(v) the structure of all final compounds was assured by at least one of the following techniques: MS or proton nuclear magnetic resonance (1H NNM) spectrometry, and the purity was assured by at least one of the following techniques: TLC or HPLC;

(vi) 1H NMR spectra were recorded on either a Varian Unity or a Varian Inova instrument at 500 or 600 MHz using the indicated solvent; when line-listed, NMR data is in the form of delta values for major diagnostic protons, given in parts per million (ppm) relative to residual solvent peaks (multiplicity and number of hydrogens); conventional abbreviations used for signal shape are: s. singlet; d. doublet (apparent); t. triplet (apparent); m. multiplet; br. broad; etc.;

(vii) MS data were recorded on a Waters Micromass unit, interfaced with a Hewlett-Packard (Agilent 1100) HPLC instrument, and operating on MassLynx/OpenLynx software; electrospray ionization was used with positive (ES+) or negative ion (ES−) detection; the method for LCMS ES+ was 1-2 mL/min, 10-95% B linear gradient over 5.5 min (B=0.05% TFA-acetonitrile, A=0.05% TFA-water), and the method for LCMS ES− was 1-2 mL/min, 10-95% B linear gradient over 5.5 min (B=0.1% formic acid—acetonitrile, A=0.1% formic acid—water), Waters XTerra C18-3.5 um-50×3.0 mmID and diode array detection;

(viii) automated purification of compounds by preparative reverse phase HPLC was performed on a Gilson system using a YMC-Pack Pro C18 column (150×20 mm i.d.) eluting at 20 mL/min with 0-50% acetonitrile in water (0.1% TFA);

(ix) column chromatography was carried out on a glass silica gel column using Kieselgel 60, 0.063-0.200 mm (Merck), or a Biotage cartridge system;

(x) chemical symbols have their usual meanings; the following abbreviations have also been used v (volume), w (weight), b.p. (boiling point), m.p. (melting point), L (litre(s)), mL (millilitres), g (gram(s)), mg (milligrams(s)), mol (moles), mmol (millimoles), eq or equiv (equivalent(s)), IC50 (molar concentration which results in 50% of maximum possible inhibition), EC50 (molar concentration which results in 50% of maximum possible efficacy), uM (micromolar), nM (nanomolar).

(xi) definitions and acronyms are as follows:
DIBALH is diisobutyl aluminum hydride;
HOBt is N-hydroxy benzotriazole;
DCC is dicyclohexyl carbodiimide;
THF is tetrahydrofuran;
DMF is dimethylformamide;
DCM is dichloromethane (methylene chloride);
OTf is triflate;
TFA is trifluoroacetic acid;
EDC is 1-ethyl-3-(3-dimethylaminopropyl)carbodiimide hydrochloride;
LDA is lithium diisopropyl amide;
TEMPO is 2,2,6,6-tetramethyl-1-piperidinyloxy, free radical;
DMSO is dimethylsulfoxide;
Comins' Reagent is 2-[N,N-Bis(trifluoromethylsulfonyl)amino]-5-chloropyridine;
Burgess Reagent is (methoxycarbonylsulfamoyl)triethylammonium hydroxide-inner salt;
KHMDS is potassium hexamethyldisilazane;
DMAP is N,N-dimethyl-4-aminopyridine;
NMM is N-methylmorpholine;
TrisylN3 is triisopropylphenyl azide;
IPA is isopropyl alcohol;
PMBOH is paramethoxybenzyl alcohol;
CDI is carbonyl diimidazole.

### Example 1

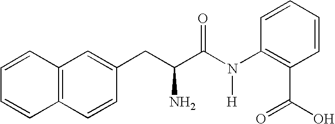

Commercially available N-(tert-butoxycarbonyl)-3-(2-naphthyl)L-alanine (500 mg, 1.58 mmol) in 10 mL of CH₂Cl₂ was cooled to −10° C. and DCC (394 mg, 1.9 mmol) followed by HOBT (215 mg, 1.59 mmol) were added. The reaction mixture was stirred for 1 h, and ethyl 2-aminobenzoate (263 mg, 1.59 mmol) was added. The reaction mixture was allowed to warm to room temperature and stirred for 12-24 h. Upon completion, a saturated solution of sodium bicarbonate (50 mL) was added, and the biphasic mixture was allowed to stir for 10 minutes. The organic layer was separated, dried over sodium sulfate, concentrated in vacuo, and purified by flash chromatography (Biotage 40M) to give the desired product. To a solution of amide (420 mg, 0.90 mmol) in 5 mL of THF/MeOH/H₂O (2:5:1), was added potassium hydroxide (153 mg, 2.72 mmol). The biphasic solution was allowed to stir for 12 h. Following completion, the reaction was concentrated in vacuo, diluted with 10 mL of water, cooled to 0° C. and acidified with concentrated HCl to a pH of 3. The acidic solution was extracted three times with ethyl acetate (10 mL), and the organic extracts were dried with sodium sulfate and concentrated in vacuo. Without further purification, the anthranilic acid (391 mg, 0.9 mmol) was diluted with 4 ml of CH₂Cl₂/trifluoracetic acid (1:1) and allowed to stir at room temperature for 4 h. Upon completion, the reaction mixture was concentrated and purified by preparative reverse phase HPLC on a Gilson system to afford the desired product. ¹H NMR (CD₃OD, 500 MHz) δ 8.51 (d, 1H), 7.99 (d, 1H), 7.81 (m, 2H), 7.74 (m, 2H), 7.57 (t, 1H), 7.45 (m, 2H), 7.39 (d, 1H), 7.17 (t, 1H), 4.41 (m, 1H), 3.43 (m, 2H); LCMS m/z 335 (M+H).

### Example 2

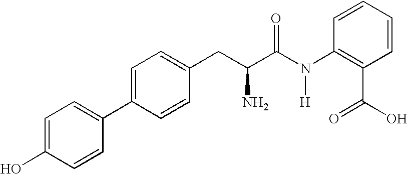

Commercially available N-(tert-butoxycarbonyl)-p-iodo-L-phenylalanine (2 g, 5.11 mmol) in 50 mL of CH₂Cl₂ was cooled to −10° C., and DCC (1.26 g, 6.1 mmol) followed by HOBT (828 mg, 6.13 mmol) were added. The reaction mixture was stirred for 1 h and ethyl 2-aminobenzoate (1.01 g, 6.13 mmol) was added. The reaction mixture was allowed to warm to room temperature and stirred for 12-24 h. Upon completion, a saturated solution of sodium bicarbonate (50 mL) was added, and the biphasic mixture was allowed to stir for 10 minutes. The organic layer was separated, dried over sodium sulfate, concentrated in vacuo, and purified by flash chromatography (Biotage 40M) to give the desired product. To a degassed solution of the amide (100 mg, 0.18 mmol) in 1 mL of dioxane was added 4-hydroxyphenylboronic acid (103 mg, 0.74 mmol), triethylamine (74 mg, 0.74 mmol), and tetrakis-triphenylphosphine palladium (21.4 mg, 0.02 mmol). The resulting mixture was heated in the microwave for 10 minutes at 100° C. Following the reaction completion, the mixture was concentrated in vacuo, and purified by flash chromatography (Biotage 40S) to give the desired product. To a solution of the amide (94 mg, 0.19 mmol) in 5 mL of THF/MeOH/H₂O (2:5:1), was added lithium hydroxide (91 mg, 3.8 mmol). The biphasic solution was allowed to stir for 12 h. Following the completion, the reaction was concentrated in vacuo, diluted with 10 mL of water, cooled to 0° C. and acidified with concentrated HCl to a pH of 3. The acidic solution was extracted three times with ethyl acetate (10 mL) and the organic extracts were dried with sodium sulfate and concentrated in vacao. Without further purification, the anthranilic acid (90 mg, 0.19 mmol) was diluted with 4 ml of CH₂Cl₂/trifluoracetic acid (1:1) and allowed to stir at room temperature for 4 h. Upon completion, the reaction mixture was concentrated and purified by preparative reverse phase HPLC on a Gilson system to afford the desired product. ¹H NMR (CD₃OD, 500 MHz) δ 8.52 (m, 1H), 8.05 (m, 1H), 7.58 (m, 1H), 7.48 (d, 2H), 7.38 (m, 2H), 7.29 (d, 2H), 7.20 (t, 1H), 6.83 (m, 2H), 4.30 (m, 1H), 3.28 (m, 2H); LCMS m/z 377 (M+H).

### Example 3

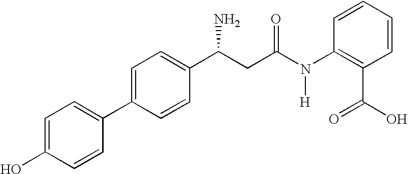

Commercially available (R)—N—BOC-3-amino-3-(4-bromophenyl)propanoic acid (500 mg, 1.45 mmol) was dissolved in anhydrous methylene chloride under argon atmosphere at 0° C. The solution was treated with methanesulfonyl chloride (0.12 mL, 1.45 mmol) and 4-dimethylaminopyridine (444 mg, 3.63 mmol), and was maintained at 0° C. for 15 min. Upon the addition of benzyl anthranilate (330 mg, 1.45 nmol), the solution was heated to 45° C. for 15 h. The reaction mixture was partitioned between water and ethyl acetate, the organic phase separated, dried over anhydrous sodium sulfate, and evaporated under reduced pressure. The crude product was purified by preparative RPHPLC. This intermediate (40 mg, 0.08 mmol) was dissolved in degassed anhydrous DMF under argon atmosphere. To this solution was added 4-methoxyphenylboronic acid (19 mg, 0.12 mmol), degassed aqueous 2M Na₂CO₃ (0.08 mL, 0.16 nmol), Pd(dba)₃ (4 mg), P-(Tos)₃ (2.5 mg). Microwave conditions (250 psi, 150 W, 100° C.) were used to heat the reaction mixture for 20 min. The reaction mixture was cooled, and partitioned between pH 7 buffer and ethyl acetate. The organic phase was then separated, dried, and concentrated in vacuo. Preparative RPHPLC afforded the product. This biphenyl intermediate (20 mg, 0.04 mmol) was combined with anhydrous methylene chloride and BBr₃ (0.4 mL, 0.40 mmol) at 0° C. The solution was allowed to slowly wann to room temperature and was monitored by LCMS. After 1 hour, the reaction mixture was partitioned between pH 7 Buffer and ethyl acetate, dried, and evaporated under reduced pressure. The desired product was purified by preparative RPHPLC. ¹H NMR (DMSO-d₆, 500 MHz) (11.71(s, 1H), 8.81 (s, 1H), 8.85 (s, 2H), 7.53 (d, 1H), 7.11(d, 1H), 6.57 (s, 4H), 6.63-6.58 (m, 3H), 6.23 (t, 1H), 5.98 (d, 2H), 3.92 (t, 1H), 2.57-2.34 (m, 2H); LCMS m/z 377 (M+H).

### Example 4

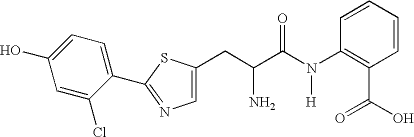

Commercially available 2-bromo-5-formylthiazole (5 g, 26 mmol) in tetrahydrofuran (50 mL) was cooled to 0° C. To this solution was added portionwise, sodium borohydride (1.23 g, 32 mmol), and the reaction mixture was stirred for 1 h at 0° C., and then allowed to warm to room temperature and stirred for another hour. Upon reaction completion, water (100 ml) was added and the mixture was allowed to stir for 30 minutes. The reaction mixture was concentrated in vacuo and purified via flash chromatography (Biotage 40M). To the corresponding thiazole-alcohol (3.87 g, 20 mmol) in CH₂Cl₂ (100 mL) at 0° C. was added carbon tetrabromide (13.2 g, 40 mmol) and triphenylphosphine (10 g, 40 mmol). The reaction mixture was allowed to stir at room temperature for 1 h. The mixture was concentrated in vacuo and purified via flash chromatography (Biotage 40 M). To a pre-cooled (0° C.) solution containing commercially available ethyl N-(diphenylmethylene) glycinate (2.87 g, 10.7 mmol) in tetrahydrofuran (18 mL), was added potassium tert-butoxide (1.2 g, 10.7 mmol) in tetrahydrofuran (25 mL). The reaction mixture was stirred at this temperature for 30 minutes and cooled to −78° C. To this pre-cooled (−78° C.) solution was added the thiazolyl bromide (1.83 g, 7.1 mmol) in tetrahydrofuran (8 mL). The reaction mixture was stirred at this temperature for 30 minutes, and then allowed to stir at room temperature for 1 h. A saturated solution of ammonium chloride (40 mL) was then added, the organic layer was separated, and the aqueous layer was extracted with ethyl acetate (2×50 mL). The organic layers were combined, dried over sodium sulfate, concentrated in vacuo, and purified by flash chromatography (Biotage 40M). To the corresponding Schiff base (3.17 g, 7.1 mmol) was added concentrated hydrochloric acid (9 mL), and the reaction mixture was allowed to stir for 1 h at room temperature. Following the completion of the reaction, the aqueous layer was washed 3 times with ethyl acetate (20 mL), and the aqueous layer was concentrated in vacuo. Without further purification, the amine (1.99 g, 7.16 mmol) in CH₂Cl₂ (100 mL) was treated with triethylamine (2.89 g, 29 mmol) and di-tert-butyl dicarbonate (3.1 g, 14.3 mmol). The reaction mixture was stirred for 12 h at room temperature. Upon reaction completion, a saturated solution of sodium bicarbonate (100 mL) was added, and the mixture was allowed to stir for 30 minutes. The organic layer was separated, and the aqueous layer was extracted with CH₂Cl₂ (2×50 mL). The organic layers were combined, dried over sodium sulfate, concentrated in vacuo, and purified by flash chromatography (Biotage 40 M). To the amino acid (0.82 g, 2.1 mmol) in toluene (20 mL) was added (2-chloroA-methoxyphenyl)boronic acid (0.81 g, 4.3 mmol), tetrakis-triphenylphosphine palladium (0.12 g, 0.1 mmol), and potassium carbonate (0.89 g, 6.4 mmol). The reaction mixture was heated to 100° C. for 12 h. Following the reaction completion, the mixture was concentrated in vacuo and purified via flash chromatography (Biotage 40M). To the desired amino acid (0.57 g, 1.3 mmol) in tetrahydrofuran (6 mL) was added water (6 mL), methanol (1 mL), and lithium hydroxide (0.12 g, 5.2 mmol). The biphasic reaction mixture was allowed to stir at room temperature for 12 h. The mixture was concentrated in vacuo, diluted with 10 mL of water, cooled to 0° C. and acidified with concentrated HCl to a pH of 3. The acidic solution was extracted three times with ethyl acetate (10 mL), and the organic extracts were dried with sodium sulfate and concentrated in vacuo. Without further purification, the carboxylic acid (0.14 g, 0.33 mmol) in tetrahydrofuran (5 mL) at −20° C. was treated with 4-methylmorpholine (0.067 g, 0.67 mmol), followed by the dropwise addition of isobutyl chloroformate (0.045 g, 0.33 mol). The reaction mixture was stirred for 10 minutes, followed by the addition of ethyl-2-aminobenzoate (0.11 g, 0.67 mmol). The mixture was stirred at −20° C. for 2 h and then room temperature for 12 h. Following the reaction completion, the precipitate was filtered off and the filtrate was concentrated in vacuo and purified via flash chromatography (Biotage 40S). To the purified anthranilic acid derivative (18 mg, 33 mmol) in CH₂Cl₂ (3 mL) at 0° C., was added borontribromide (1M, 0.33 mmol). The mixture was allowed to stir at 0° C. for 10 minutes and then room temperature for 1 h. Following the reaction completion, water (10 mL) was added, and the biphasic mixture was stirred for 10 minutes. The reaction mixture was then concentrated in vacuo, diluted with 10 mL of water, cooled to 0° C. and basified with sodium hydroxide to a pH of 14. The basic reaction mixture was allowed to stir for 12 h at room temperature. The mixture was concentrated in vacuo and then diluted with water (2 mL). The aqueous solution was acidified with concentrated hydrochloric acid (pH=3) and then purified by reverse phase HPLC (Gilson) to provide the desired racemnic product. ¹H NMR (CD₃OD, 500 MHz) δ 8.53 (d, 1H), 8.09 (d, 1H), 7.84 (d, 1H), 7.7 (s, 1H), 7.61 (m, 1H), 7.23 (m, 1H), 6.91 (d, 1H), 6.81 (m, 1H), 4.43 (m, 1H), 3.60 (m, 2H); LCMS m/z 418 (M+H).

### Example 5

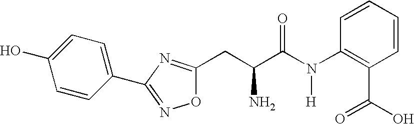

To the commercially available N′-hydroxy-4-methoxybenzenecarboximidamide (1.2 g, 7.23 mmol) and Fmoc-tert-butoxy-aspartic acid (2.4 g, 6.0 mmol) in CH₂Cl₂/DMF (15 mL, 9:1) at −10° C., was added HOBT (0.98 g, 7.2 mmol) and DCC (1.49 g, 7.2 mmol). The reaction mixture was stirred at this temperature for 20 minutes and then stirred at room temperature for 3 h. Following the reaction completion, the solution was concentrated in vacuo, diluted with ethyl acetate (50 mL), washed with a saturated solution of sodium bicarbonate (50 mL), dried over sodium sulfate, and concentrated in vacuo. Without further purification, the aspartic acid derivative (3.37 g, 6.02 mmol) in ethanol (20 mL), was treated with sodium acetate (0.49 g, 6.02 mmol) in water (2 mL). The reaction mixture was then heated for 3 h at 86° C. The mixture was concentrated and purified via flash chromatography (Biotage 40M). To the purified oxadiazole (2.39 g, 4.3 mmol) in CH₂Cl₂ (5 mL) was added trifluoroacetic acid (2 mL), and the mixture was allowed to stir for 3 h at room temperature. At this time, the reaction mixture was concentrated, and the crude acid (1.0 g, 2.15 mmol) in toluene (10 mL) was subjected to thionyl chloride (2 mL). The reaction mixture was heated to 95° C. for 2 h. Following the completion of the reaction, the solution was concentrated, diluted with CH₂Cl₂ (10 mL), and ethyl aminobenzoate (1.1 g, 6.8 mmol) was added dropwise. The reaction mixture was allowed to stir at room temperature for 12 h, at which time the mixture was quenched with a saturated solution of sodium bicarbonate (20 mL) and allowed to stir for 20 minutes. The organic layer was isolated, dried over sodium sulfate, concentrated in vacuo, and purified by flash chromatography (Biotage 41M). To the pure anthranilic acid derivative (0.17 g, 0.27 mmol) in CH₂Cl₂ (5 mL) cooled to 0° C., was added a solution of borontribromide (1M, 2.68 mmol). The reaction mixture was allowed to stir at room temperature for 2 h. At this time, the reaction mixture was concentrated in vacuo, diluted with water (3 mL) and basified with solid sodium hydroxide (pH=13). The basic solution was allowed to stir at room temperature for 12 h. The aqueous solution was acidified (pH=3) with concentrated hydrochloric acid, and purified by reverse phase HPLC (Gilson) to afford the desired product. ¹H NMR (CD₃OD, 500 MHz) δ 8.49 (d, 11H), 8.1 (d, 1H), 7.86 (d, 2H), 7.60 (t, 11H), 7.23 (t, 1H), 6.86 (d, 21), 4.73 (t, 2H), 3.73 (m, 1H); LCMS m/z 369 (M+H).

### Example 6

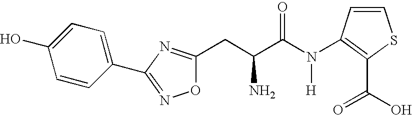

Example 6 was generated under similar reaction conditions described in the examples above and shown in Scheme 4. Example 6 utilized commercially available methyl 3-amino-2-thiophenecarboxylate (Aldrich) as a starting material to obtain the desired product. ¹H NMR (CD₃OD, 500 MHz) δ 8.00 (d, 1H), 7.99 (d, 2H), 7.70 (d, 1H), 6.88 (d, 2H), 4.80 (m, 1H), 3.67 (m, 2H); LCMS m/z 375 (M+H).

### Example 7

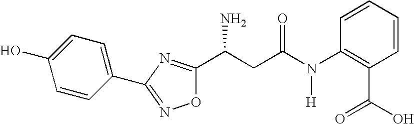

Example 7 was generated under similar reaction conditions described in the examples above and shown in Scheme 4. Example 7 utilized commercially available orthogonally protected Fmoc-D-Asp (OtBu)-OH (Advanced Chemtech) as a starting material to obtain the desired product. ¹H NMR (CD₃)₂SO, 500 MHz) δ 11.26 (s, 1H), 10.2 (s, 1H), 8.30 (m, 1H), 7.98 (m, 1H), 7.85 (m, 2H), 7.58 (m, 1H), 7.20 (m, 1H), 6.93 (m, 2H), 5.21 (m, 1H), 3.17 (m, 2H); LCMS m/z 369 (M+H).

### Example 8

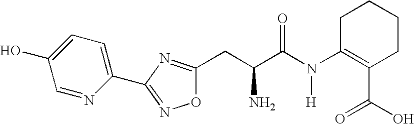

To a mixture of 5-bromo-2-cyanopyridine (1 g, 5.5 mmol), cesium carbonate (3.6 g, 11 mmol), 4-methoxybenzyl alcohol (1.5 g, 10.9 mmol) in a solution of 20 mL of toluene, was quickly added 1,10-phenanthroline (98 mg, 0.55 miol) and copper(I) iodide (52 mg, 0.27 mmol) under nitrogen. The reaction mixture was heated at 120° C. overnight. To the mixture was then added water (150 mL), and partitioned twice with ethyl acetate (2×100 mL). The aqueous layer was then extracted twice with dichloromethane (2×100 mL). The combined organic phases were dried with sodium sulfate and concentrated in vacuo. The residue was dissolved in DMSO and purified by RPHPLC to give 5-(4-methoxybenzyloxy)-2-cyanopyridine as a pale yellow solid. To a slurry of this intermediate (60 mg, 0.25 mmol) and hydroxylamine hydrochloride (38 mg, 0.55 mmol) in 8 mL of ethanol, was added 0.17 mL of 3 N sodium hydroxide aqueous solution. The reaction mixture was stirred at 23° C. overnight. The residue was purified by RPHPLC to give 5-(4-methoxybenzyloxy)-2-hydroxyamidinylpyridine as a white solid. To the commercially available Boc-tert-butoxy-aspartic acid (10.0 g, 35 mmol) in CH₂Cl₂ (100 μL) was added CDI (11 g, 69 mmol). The reaction mixture was stirred at room temperature for 1 hour and then the corresponding N′-hydroxy-pyridinecarboximidamide prepared above (19.0 g, 69 mmol) was added. The reaction was allowed to stir for 2 hours, at which time the reaction was filtered, and the organic layer was washed with saturated ammonium chloride (100 mL), dried over sodium sulfate, and concentrated in vacuo. Without further purification, the aspartic acid derivative (5.0 g, 9.1 mmol) in toluene (50 mL) was heated at 130° C. for 16 hours. The mixture was concentrated in vacuo and purified via flash chromatography (Biotage 40M). To a solution of the oxadiazole (3.71 mg, 7.0 mmol) in 50 mL of THF/MeOH/H₂O (2:5:1), was added sodium hydroxide (0.84 g, 21 mmol). The biphasic solution was allowed to stir for 12 h. The mixture was concentrated in vacuo, diluted with 10 mL of water, cooled to 0° C. and acidified with concentrated HCl to a pH of 3. The acidic solution was extracted three times with ethyl acetate (20 mL) and the organic extracts were dried with sodium sulfate and concentrated in vacuo. Without further purification, the acid (1.77 g, 3.76 mmol) in CH₂Cl₂ (50 μL), was treated with N-hydroxysuccinimmide (649 mg, 5.64 mmol) and EDC (1.09 g, 5.64 mmol). The reaction mixture was allowed to stir for 4 hours and then diluted with ethyl acetate (100 mL). The mixture was filtered, the organic layer washed with water (3×50 mL), dried over sodium sulfate and concentrated in vacuo. The activated ester was diluted with dioxane (100 mL), ammonium hydroxide (10 mL) was added, and the reaction mixture was allowed to stir for 1 hour. Following the completion of the reaction, the organic layer was isolated, dried over sodium sulfate and concentrated in vacuo and purified via flash chromatography (Biotage 40 M). To the purified amide (0.32 g, 0.69 mmol) in a degassed solution of dioxane (7 mL) was added the corresponding triflate (0.26 g, 0.83 mmol), cesium carbonate (0.32 g, 0.97 mmol), xantphos ligand (0.8 g, 0.13 mmol), and Pd₂(dba)₃ catalyst (0.6 g, 0.07 mmol), and the reaction mixture was heated to 75° C. for 6 hours. The mixture was cooled, filtered, concentrated in vacuo, and purified via flash chromatography (Biotage 40 M). To the desired cycloalkene (0.10 g, 0.1 mmol) in CH₂Cl₂ (5 mL) at 0° C. was added triethylsilane (0.1 mL) and trifluoroacetic acid (1 mL). The reaction mixture was allowed to stir for 4 hours at room temperature. The mixture was neutralized with a saturated solution of sodium bicarbonate (5 mL), the organic layer was separated, dried over sodium sulfate and concentrated in vacuo. The amine, in tetrahydrofuran (2 μL) at 0° C., was then treated with methanol (1 mL) and a 1M solution of lithium hydroxide (1 mL). The reaction mixture was allowed to stir for 6 hours. The reaction mixture was acidified to pH=2 with 2M hydrochloric acid, and the mixture purified by reverse phase HPLC (Gilson) to afford the desired product. ¹H NMR (500 MHz, (CD₃)₂SO) δ 11.6 (s, 1H), 8.54 (s, 1H), 8.28 (s, 1H), 7.93 (d, 1H), 7.34 (d, 1H), 4.55 (m, 1H), 3.59 (m, 2H), 2.75 (m, 2H), 2.24 (m, 2H), 1.56 (m, 4H); LCMS m/z 396 (M+Na).

### Example 9

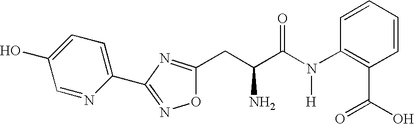

Example 9 was generated under similar reaction conditions described in the examples above and shown in Scheme 4. Example 9 utilized the 5-(4-methoxybenzyloxy)-2-hydroxy-amidinylpyridine (also shown in Scheme 5) as an intermediate to obtain the desired product. ¹H NMR (DMSO-d₆, 500 MHz) δ 11.32 (s, 1H), 8.62 (s, 1H), 8.28 (m, 1H), 8.21 (d, 1H), 7.98 (d, 1H), 7.90 (d, 1H), 7.66 (t, 1H), 7.31 (m, 2H), 4.70 (m, 1H), 3.65 (m, 2H); LCMS m/z 370 (M+H).

### Example 10

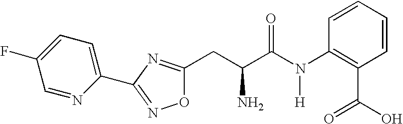

Example 10 utilized a 5-fluoro-2-hydroxyamidinylpyridine as an intermediate to obtain the desired product. To a mixture of 5-amino-2-yanopyridine (100 g, 840 mmol) cooled to −10° C. was added HF-pyridine (500 mL, 70% v/v). Sodium nitrite (91 g, 1.32 mol) was added in portions. The reaction was then stirred at −10° C. for 45 minutes, room temperature for 30 minutes, and 80° C. for 90 minutes. Upon completion, the reaction was cooled to room temperature and quenched with ice/water. The aqueous solution was extracted with CH₂Cl₂, dried over magnesium sulfate and concentrated. The fluoropyridine (40 g, 328 mmol) was treated with sodium carbonate (82 g, 773 mmol) and hydroxylamine-hydrochloride (45 g, 652 mmol) in methanol (300 mL). The reaction was allowed to stir for 24 h and upon completion, the reaction was concentrated in vacuo, diluted with water, filtered and dried under vacuum.

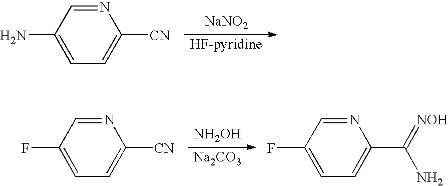

Example 10 was generated under similar reaction conditions described in the examples above and shown in Schemes 4 and 5. ¹H NMR (DMSO-d₆, 500 MHz) δ 12.0 (s, 1H), 8.79 (s, 1H), 8.23 (m, 1H), 8.14 (m, 1H), 7.97 (m, 1H), 7.64 (m, 1H), 7.26 (m, 1H), 4.64 (m, 1H), 3.56 (m, 2H); LCMS m/z 394 (M+Na).

### Example 11

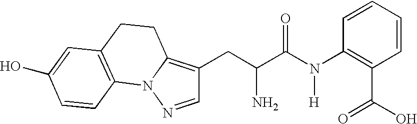

Commercially available ethylacetoacetate (10 g, 77 mmol) in 100 ml of THF was cooled to −78° C. Lithium diisopropylamide (2M, 153.6 mmol) was added dropwise, and the reaction mixture was allowed to stir at low temperature for 1 h. To this reaction mixture was added a solution of 2-bromo-5-methoxybenzyl bromide (24 g, 84 mmol) in 100 mL of THF. The reaction mixture was allowed to warm to room temperature and stir for 4 h. Upon reaction completion, a saturated solution of ammonium chloride (IL) was added and the biphasic mixture was allowed to stir for 30 minutes. The mixture was extracted three times with CH₂Cl₂ (100 mL), the organic layers were combined, dried over sodium sulfate, concentrated in vacuo, and purified using flash chromatography (Biotage 40M). To the purified ester (30 g, 91.7 mmol) was added triethylorthoformate (20.4 g, 138 mmol) and acetic anhydride (50 mL). The mixture was heated at 120° C. for 3 h. Following reaction completion, the reaction mixture was partitioned between ethyl acetate (100 mL) and saturated sodium bicarbonate (100 mL). The aqueous solution was further extracted with ethyl acetate (3×100 mL), the organic phase was combined, dried over sodium sulfate and concentrated in vacuo. To the crude ester (35 g, 92 mmol) was added ethanol (100 mL) followed by a solution of hydrazine hydrochloride (12.5 g, 183 mmol) in water (10 mL) and the reaction mixture was refluxed for 2 h. Upon reaction completion, the solution was concentrated in vacuo, diluted with ethyl acetate (100 mL), washed with saturated sodium bicarbonate (3×50 mL), dried with sodium sulfate, concentrated in vacuo and purified via flash chromatography (Biotage 40M). To the corresponding pyrazole (23 g, 9.2 mmol) in degassed toluene (20 mL) was added copper iodide (0.087 g, 0.46 mmol), potassium carbonate (3.81 g, 27.6 mmol), and dimethylethylenediamine (162 mg, 1.84 mmol). The reaction mixture was heated at 110° C. for 12 h. Upon reaction completion, the mixture was concentrated in vacuo, diluted with ethyl acetate and washed with 1M HCl (100 mL). The organic phase was dried over sodium sulfate, concentrated in vacuo, and purified by flash chromatography (Biotage 40M). The purified ester (656 mg, 2.42), in toluene (10 mL) was cooled to −78° C., and DIBALH (1M, 4.82 mmol) was added dropwise. The reaction mixture was warmed to room temperature and allowed to stir for 2 h. Following reaction completion, the mixture was quenched at 0° C. with 1M HCl (50 mL). The aqueous layer was extracted with ethyl acetate (3×20 mL), the organic layers were combined, dried over sodium sulfate, and concentrated in vacuo. The crude alcohol was purified via flash chromatography (Biotage (40M). To the pure alcohol (537 mg, 2.33 mmol) in CH₂Cl₂ (10 mL) at 0° C. was added iodobenzene diacetate (1.33 g, 4.15 mmol) and TEMPO (43 mg, 0.28 mmol). The reaction mixture was allowed to stir for 4 h at room temperature. Following reaction completion, the mixture was quenched with saturated sodium bicarbonate (20 mL), and the aqueous layer was extracted with CH₂Cl₂ (3×10 mL). The organic layers were combined, dried over sodium sulfate, concentrated in vacuo, and purified via flash chromatography (Biotage 40M). The corresponding aldehyde (508 mg, 2.23 mmol) was added dropwise in tetrahydrofuran (10 mL) to a premixed solution of sodium hydride (80 mg, 3.34 mmol) and trimethyl phosphonoacetate (608 mg, 3.34 mmol) at 0° C. The reaction mixture was allowed to stir for 3 h at room temperature. Upon reaction completion, the reaction mixture was concentrated and purified via flash chromatography (Biotage 40M). To the purified acetate (688 mg, 2.41 mmol) in 4 ml of MeOH/CH₂Cl₂ (3:1) was added 68 mg of 10% palladium hydroxide. The heterogenous reaction mixture was charged with a balloon of hydrogen gas and allowed to stir at room temperature for 5 h. The reaction mixture was filtered, the filtrate was concentrated and purified via flash chromatography (Biotage 40M). To a pre-cooled (−78° C.) solution of the purified ester (409 mg, 1.43 mmol), in 10 ml THF was added potassium hexamethyldisilane (0.5M, 2.86 mmol). The reaction mixture was stirred at −78° C. for 30 minutes at which time trisylazide (885 mg, 2.86 mmol) in THF (10 mL) was added dropwise. The mixture was allowed to stir at low temperature for 10 minutes followed by the addition of acetic acid (172 mg, 2.86 mmol). The reaction mixture was warmed to room temperature and allowed to stir for 2 h. After the reaction was complete, CH₂Cl₂ (50 mL) was added and the organic layer was washed with saturated sodium bicarbonate (50 mL). The organic phase was dried over sodium sulfate, concentrated in vacuo, and purified (Biotage 40M). To the pure azide (468 mg, 1.43 mmol) in 5 mL of THF/water (2:1) at room temperature was added lithium hydroxide (137 mg, 5.72 mmol). The biphasic mixture was stirred for 12 h at room temperature. Upon completion, the reaction mixture was concentrated in vacuo, diluted with 10 mL of water, cooled to 0° C. and acidified with concentrated HCl to a pH of 3. The acidic solution was extracted three times with ethyl acetate (10 mL), and the organic extracts were dried with sodium sulfate and concentrated in vacuo. Without further purification, the acid (201 mg, 0.64 mmol) in CH₂Cl₂ (20 ml) at 0° C. was treated with DCC (264 mg, 1.28 mmol) and HOBT (173 mg, 1.28 mmol) and allowed to stir for 1 h. Ethyl aminobenzoate (211 mg, 1.28 mmol) was subsequently added, and the reaction mixture was allowed to stir at room temperature for 18 h. Following reaction completion, a saturated solution of sodium bicarbonate was added and this mixture was allowed to stir for 30 minutes. The organic layer was then separated and the aqueous layer was extracted with CH₂Cl₂ (3×10 mL). The organic layers were combined, dried over sodium sulfate, concentrated in vacuo, and purified by flash chromatography (Biotage 40M). To the purified anthranilic acid (147 mg, 0.32 mmol) in ethanol (5 mL) was added 10% palladium on carbon (14.7 mg). The reaction mixture was charged with hydrogen gas (balloon) and allowed to stir at room temperature for 2 h. Following the reaction completion, the mixture was filtered and the filtrate was concentrated in vacuo. To the desired amine (48 mg, 0.11 mmol), without further purification, in CH₂Cl₂ (4 mL) at 0° C. was added a solution of boron tribromide (1M, 1.1 mmol). The mixture was allowed to warm to room temperature and stirred for 2 h. At this time, the mixture was quenched with water (4 mL), and allowed to stir at room temperature for 30 minutes. Upon reaction completion, the biphasic mixture was concentrated, diluted with THF/water (5 mL, 2:1), and sodium hydroxide (100 mg, 2.5 mmol) was added. The reaction mixture was stirred for 5 h at room temperature. The reaction mixture was concentrated in vacuo, diluted with 10 mL of water, cooled to 0° C. and acidified with concentrated HCl to a pH of 3. The crude residue was purified by reverse phase HPLC (Gilson) to give the desired racemic product. ¹H NMR (CD₃OD, 500 MHz) δ 8.58 (d, 1H), 8.1(d, 1H), 7.59 (m, 1H), 7.52 (d, 1H), 7.45 (s, 1H), 7.19 (t, 1H), 6.72 (m, 2H), 4.23 (m, 1H), 3.16 (m, 2H), 2.86 (m, 3H), 2.68 (m, 1H); LCMS m/z 393 (M+H).

### Example 12

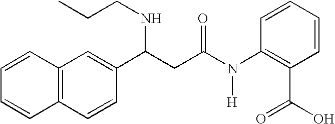

Acetic acid (1.15 g, 19.2 miol) in 140 mL of tetrahydrofuran was cooled to −78° C., and treated with lithium diisopropylamide (1.8 M, 22.2 mL, 40 mmol). The mixture was maintained for 30 min, and then commercially available 2-naphthaldehyde (2.5 g, 16.0 mmol) was added as a solution in 20 mL of tetrahydrofuran. The mixture was warmed to room temperature, aged for 3 h, partitioned between water and diethyl ether, the aqueous phase acidified with 2N HCl to pH 2, and extracted with ethyl acetate. The organic phase was concentrated in vacuo to provide the clean hydroxy acid. This intermediate (150 mg, 0.694 mmol) was dissolved in THF (5 mL) and chlorodimethoxytriazine (0.764 mmol, 134 mg) and N-methylmorpholine (0.833 mmol, 85 mg) were added. The resulting reaction mixture was allowed to stir for 1 hour at 0° C. before the addition of anthranilic acid benzyl ester (0.902 mmol, 208 mg). After the reaction mixture was warmed to room temperature over 15 hours, it was diluted with water and extracted with ethyl acetate. The combined evaporated organic residue was purified by preparatory thin layer chromatography (EtOAC, dichloromethane). This intermediate (40 mg, 0.094 mmol), was dissolved in dichloromethane (2 mL) and placed in a sealed pressure vessel. To this was added manganese dioxide (0.47 mmol, 41 mg), and the resulting reaction mixture was heated to 38° C. for 4 hours. Following filtration through Celite and concentration under reduced pressure, the residue was purified by preparatory thin layer chromatography (acetone, hexanes). This ketone (10 mg, 0.024 mmol), allylamine (0.026 mmol, 0.002 mL), and acetic acid (0.118 mmol, 0.007 mL) were dissolved in ethanol (1 mL) and the resulting reaction mixture was refluxed for 2 hours before the addition of sodium cyanoborohydride (0.048 mmol, 3 mg) in methanol (0.5 mL). This solution was then held at 45° C. for 4 days, before partition between water and ethyl acetate. Evaporation of the organic layer gave an organic residue that was purified by prep HPLC (acetonitirile-water-TFA). This allyl amine (10 mg, 0.022 mmol) was dissolved in a 1:1 mixture of dichloromethane and methanol and a catalytic amount of 20% palladium hydroxide on carbon (5 mg) was added. The reaction mixture was exposed to a hydrogen atmosphere for 3 hours before it was filtrated through Celite, concentrated under reduced pressure, and purified by prep HPLC to provide the racemic product. ¹H NMR (CD₃OD, 600 MHz) δ 8.44 (d, 1H), 8.04 (s, 1H), 8.02 (dd, 1H), 7.99 (d, 1H), 7.98-7.89 (m, 2H), 7.59 (dd, 1H), 7.56-7.54 (m, 2H), 7.51 (t, 1H), 7.13 (t, 1H), 4.96 (t, 1H), 3.42 (dd, 1H), 3.25 (dd, 1H), 3.00-2.96 (m, 1H), 2.85-2.81 (m, 1H), 1.77-1.69 (m, 2H), 0.96 (t, 3H); LCMS m/z 377 (M+H).

### Example 13

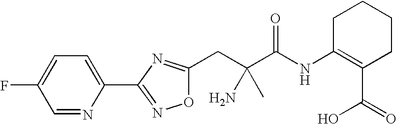

DL-α-methyl aspartic acid (1 g, 6.8 mmol) in DMSO (3 mL) hexafluoroacetone (3 eq) was added and stirred at RT for 5 h with dry ice condenser sealed. The mixture was partitioned between DCM and ice water after excess hexafluoroacetone was evaporated. The organic layer was washed with H₂O and brine to obtain the pure protected intermediate acid. EDC (331 mg, 2.0 eq, 1.728 mmol) was added to this acid (1 eq, 255 mg, 0.864 mmol) in DCM for 1 h then the fluoropyridyl hydroxyamidine (2.1 eq, 281 mg, 1.814 mmol) was added and stirred for another 2 h at RT. The reaction mixture was filtered through SiO₂, and washed with water, NH₄Cl, water, brine and dried to obtain the acylated intermediate as a crude product, which was treated with Burgess reagent (3×1 eq) in THF and heated with a microwave at 150 w, 120° C. for 3×6 min. The oxadiazole was obtained after column chromatography purification. Then NH₄OH (1 mL) was added to this protected intermediate (50 mg) in dioxane and sonicated for 1 h at RT, followed by evaporation of the solvent. This carboxamide intermediate (40 mg 1 eq, 0.121 mmol) was combined with Pd₂(DBA)₃ (0.1 eq, 11 mg), Xantphos (0.2 eq, 14 mg), CS2CO₃ (1.4 eq, 55 mg) and the required triflate described in prior examples (1.2 eq 42 mg), and the mixture in dioxane (1 mL) under N₂ was heated to 80° C. for 12 h. The mixture was cooled and diluted with CH₂Cl₂ (2 mL), and filtered through Celite. The filtrate was dried and purified by recrystallization with Et₂O/hexanes to obtain a light yellow solid. Lastly, LiOH (0.5 m, 3 eq) was added to this methyl ester (1 eq, 48 mg) in THF at 0° C. and stirred at RT for 8 h. The mixture was acidified to pH=7 with AcOH at 0° C., and the organic solvent was removed in vacuo. The crude residue was purified by HPLC to obtain the product as a white solid. ¹H NMR, CD₃OD a 8.67(d, 1H), 8.25 (dd, 1H), 7.86 (t, 1H), 3.76 (q, 2H), 3.31 (s, 3H), 2.31 (m, 2H), 1.69 (m, 2H), 1.64 (m, 4H); LCMS m/z 388 (M−H).

### Example 14

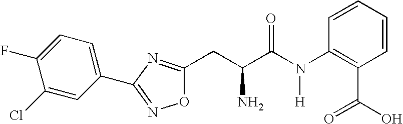

The preparation of Example 14 followed similar procedures described above. ¹H NMR, CD₃OD δ 8.52(d, 1H), 8.16 (dd, 1H), 8.12 (d, 1H), 8.03 (m, 1H), 7.61 (t, 1H), 7.41 (t, 1H), 7.25 (t, 1H), 4.75 (t, 1H), 3.76 (dq, 2H); LCMS m/z 405 (M+H).

### Example 15

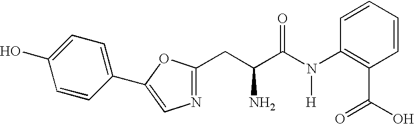

The preparation of Example 15 followed similar procedures described above, as illustrated in Scheme 9. ¹H NMR, CD₃OD δ 8.53(d, 1H), 8.09 (dd, 1H), 7.60 (t, 1H), 7.42 (d, 2H), 7.23 (t, 1H), 7.23 (s, 1H), 6.78 (d, 2H), 4.65 (t, 1H), 3.60 (dq, 2H); LCMS m/z 368 (M+H).

### Example 16

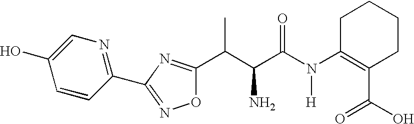

At −78° C., LiHMDS (2.25 eq, 53.42 mmol, 1 M/THF) was added to the diester of aspartic acid (1 eq. 8.005 g, 23.74 mmol) in THF (100 mL) and aged for 30 min under N₂. The solution was treated with MeI (1.2 eq. 4.05 g, 28.49 mmol), and this solution was stirred at −78° C. for another 6 h. The solution was quenched with saturated NH₄Cl (aq) solution at low temperature and extracted with AcOEt (3×100 mL). The combined organic layer was dried and purified by column chromatography to obtain both monomethylated and dimethylated products. Pd/C (˜100 mg) was added to the monomethylated intermediate (5 g) in MeOH and then hydrogenated for 16 h to obtain the mono acid product intermediate. Example 16 was subsequently synthesized following similar reaction conditions described in the examples above. ¹H NMR, Cf)₃OD δ 8.28 (d, 1H), 8.08 (d, 1H), 7.39 (dd, 1H), 4.50 (d, 1H), 3.90 (m, 1H), 2.85 (m, 2H), 2.34 (br, 2H), 1.68 (m, 4H), 1.62 (d, 3H); LCMS m/z 386 (M−H).

### Example 17

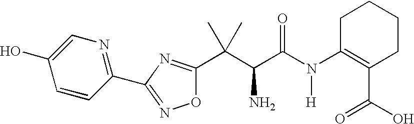

Example 17 was obtained in a similar manner as described for Example 16 above when using the dimethylated aspartate intermediate. ¹H NMR, CD₃OD δ 8.28 (d, 1H), 8.08 (d, 1H), 7.41 (dd, 1H), 4.43 (s, 1H), 2.85 (m, 2H), 2.34 (br, 2H), 1.69 (d, 6H), 1.60 (m, 4H); LCMS m/z 424 (M+Na).

### Example 18

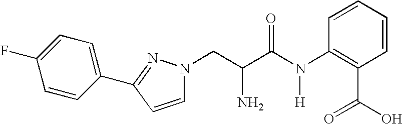

The parafluorophenyl pyrazole (200 g) and propargylate (1 g) were mixed and heated to 90° C. for 15 h, dried in vacuo to obtain a crude mixture of products, which were hydrogenated in MeOH/Pd/C at RT for 16 h to obtain the saturated ester intermediate after filtration and removal of solvent in vacuo. Then KHMDS (2 eq, 0.5 M, 8.54 mL) was added to this ester (530 mg) in THF (20 mL) at −78° C. and stirred for 30 min. Trisylazide (2 eq, 1.321 g) in THF (10 mL) was added. The mixture was allowed to stir at −78° C. for 10 min followed by addition of acetic acid (2 eq, 0.244 mL). The solution was warmed to RT overnight, and CH₂Cl₂ was added, and then washed with NaHCO₃, followed by water. The product was purified by Biotage (25S) hexane/AcOEt 10-20% to obtain the azidoester as a colorless oil. This oil was dissolved in MeOH and Pd/C was added under N2, followed by a balloon hydrogenation for 16 h to obtain the α-amino-methyl ester. This intermediate (260 mg) was dissolved in 7 N NH₃/MeOH (8 mL) and heated to 52° C. for 5 h, and the solvent removed in vacuo to obtain the amino carboxamide. This intermediate was elaborated into Example 18 under similar reaction conditions described above. ¹H NMR, CD₃OD δ 8.44 (d, 1H), 8.07 (dd, 1H), 7.75 (dd, 2H), 7.66 (dd, 1H), 7.57 (t, 1H), 7.21 (t, 1H), 7.07 (t, 2H), 6.59 (d, 1H), 4.80 (m, 2H), 4.69 (t, 1H); LCMS m/z 369 (M+H).

### Example 19

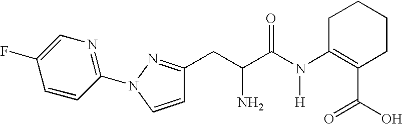

The fluoro bromopyridine (1 eq, 1 g), pyrazole (4 eq, 5.023 g), ligand (0.2 eq, 0.196 g), Cu₂O (0.05 eq, 51 mg) and Cs₂CO₃ (2 eq, 4.65 g) were mixed in CH₃CN (8 mL) and heated to 82° C. in a sealed vessel for 16 h under N2. The solution was diluted with DCM and filtered through Celite, partitioned with water, and then brine. The product was evaporated in vacuo, and purified by column chromatography (SiO₂) with 10 to 20% EtOAc/hexanes to obtain the major regioisomeric product as a white solid. Then LiBH₄ (2 eq, 128 mg) was added to this ester intermediate (1 eq, 690 mg) in THF (30 mL) and heated to reflux for 15 h. Then 0.1 N HCl (a few drops) was added and stirred for 1 h, followed by a DCMIH₂O partition, and the aqueous layer was basified with NaOH to pH=9 and extracted with DCM. The combined organic phase was dried to obtain the alcohol as a white solid. Iodine (1.52 eq, 1.058 g) in AcOEt (25 ml) was added to an AcOEt (25 mL) solution of this alcohol (1 eq, 530 mg), followed by Ph₃P (1.52 eq, 1.094 g) and imidazole (1.52 eq, 0.284 g) over 10 min at RT. The solution was stirred for 1 h and washed with Na₂S₂O₃ and brine. The product was dried in vacuo, and the solid residue was extracted with Hexanes 3×70 ml and filtered. The filtration was dried to obtain the iodide product as a white solid. Then KOtBu (1.5 eq, 250 mg) was added to N-(diphenylmethylene)-glycine ethyl ester (1.5 eq, 595 mg) in THF at RT and stirred for 10 min. To this solution was added the iodide intermediate (1 eq, 450 mg) in THF (5 mL) at −78° C., and the mixture was slowly warmed to RT over 2 h. An additional 1 eq of KOtBu was added to the solution at RT and stirred for 50 h at RT. The mixture was quenched with NH₄Cl and extracted with DCM, washed with H₂O and then brine, and dried in vacuo. The residue was purified by column chromatography (hex/AcOEt-20%) to obtain the product. This intermediate (1 eq. 200 mg) was dissolved in saturated 7 N NH₃/MeOH (7 mL) solution and heated to 60° C. for 24 h in a sealed tube. The reaction mixture was dried in vacuo and, the residue was dissolved in 5 ml THF and 1 N HCl (2 mL) at RT and heated to 60° C. for 20 min. The THF was removed in vacuo. The aqueous layer was washed with Et₂O, dried in vacuo to obtain the amino carboxamide as a white solid HCl-salt. The amide intermediate (1 eq, 68 mg), triflate (1.2 eq, 82 mg), Pd₂(DBA)₃ (0.1 eq.), Xantphos (0.2 eq) and Cs₂CO₃ (2.4 eq, 186 mg) were combined in dioxane (2 mL) under N₂ and heated to 75° C. for 13 h. The mixture was cooled and diluted with CH₂Cl₂ (2 mL), filtered through Celite, and the CH₂Cl₂ removed in vacuo, and Et₂O was added to the filtrate and extracted with 3 N HCl (3×10 mL). The combined aqueous layer was basified with Na₂CO₃ to pH=9 at 0° C. and extracted with AcOEt (3×10 mL). The combined organic layer was dried in vacuo to obtain the crude product as a light yellow oil. Lastly, LiOH (0.5 M, 3 mL) was added to this ester in THF/MeOH at 0° C. and stirred for 20 h. Then AcOH was added to acidify to pH=7 at 0° C. and HPLC purification provided the product. ¹H NMR, CD₃OD δ 8.48 (d, 1H), 8.30 (d, 1H), 7.99 (dd, 1H), 1.74 (m, 1H), 6.43 (d, 1H), 4.37 (t, 1H), 3.50 (d, 2H), 2.88 (m, 2H), 2.29 (br, 2H), 1.62 (m, 4H); LCMS m/z 374 (M+H).

### Example 20

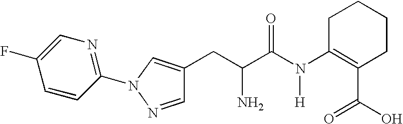

Example 20 was prepared using similar procedures described herein. ¹H NMR, CD₃OD 8.48 (s 1H), 8.30 (d, 1H), 7.95 (dd, 1H), 7.77 (dt, 1H), 7.65 (s, 1H), 4.20 (t, 1H), 3.20 (d, 2H), 2.90 (m, 2H), 2.32 (m, 2H), 1.66 (m, 4H); LCMS m/z 374 (M+H).

### Example 21

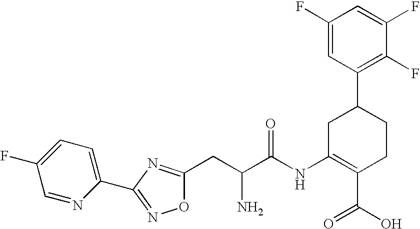

The Intermediate A was prepared as described above. The enantiomers can be resolved by chiral SFC-HPLC on a ChiralPak AS-H column using 25% MeOH/CO₂ to provide Enantiomer A as the faster eluting product after 2.1 minutes and Enantiomer B as the slower eluting product after 3.0 minutes. It is noted that basic conditions such as hydroxide may racemize the amino stereocenter, and in some cases alternate ester protection (eg. methyl versus PMB or benzyl) strategies are useful to suppress potential epimerization.

### Resolved Intermediate A

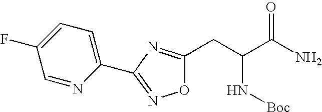

To a solution of cyclohexane 1,3-dione (1.0 g, 8.92 mmol) and 2,6-lutidine (2.07 mL, 17.84 mmol) in DCM cooled to 0° C. was added trifluoromethane sulfonic anhydride (2.25 mL, 13.38 mmol). The reaction mixture was stirred at room temperature for 30 minutes and quenched by the addition of 1N HCl. The resulting mixture was extracted with DCM. The organic layer was washed with 1N HCl, dried over anhydrous sodium sulfate, filtered and concentrated in vacuo. The residue was purified by flash chromatography using 20% ethyl acetate hexanes to give the desired product as a light brown oil. To a solution of this triflate (8.71 g, 35.7 mmol) in THF (100 mL) was added 2,3,5-trifluorophenyl boronic acid, Na₂CO₃ (50 mL, 2.0 M solution) and dichlorobis(triphenylphosphine)palladium (1.0 g). The resulting mixture was heated at 60° C. under a nitrogen atmosphere. After 30 minutes, the reaction was cooled to room temperature and diluted with ethyl acetate. The organic layer was washed with brine, dried over anhydrous sodium sulfate, filtered and concentrated in vacuo. The residue was purified by flash chromatography using 10% ethyl acetate hexanes to give the desired compound as a light yellow solid. To a solution of this intermediate (7.5 g, 33.15 mmol) in anhydrous THF cooled to −78° C. under a nitrogen atmosphere was added LHMDS (36.5 mL, 36.5 mmol, 1.0 M in THF). The reaction mixture was stirred at 0° C. for 25 minutes. It was then cooled to −78° C. and methyl cyanoformate (3.16 mL, 39.78 mmol) was added. After 30 minutes, the reaction was quenched by pouring into water (100 mL). The resulting mixture was extracted with ethyl acetate (3×). The organic layer was washed with brine dried over anhydrous sodium sulfate, filtered and concentrated in vacuo. The residue was purified by flash chromatography (silica-gel) using 10% ethyl acetate-hexanes to give the desired product as a yellow solid. To a solution of this intermediate (7.49 g, 26.37 mmol) in methanol (100 mL) was added Pd/C (100 mg, 10% by weight). The resulting reaction was stirred under H₂ balloon for 18 hours. The reaction mixture was filtered through celite. The filtrate was concentrated in vacuo and purified by flash chromatography using 10% ethyl acetate-hexanes to give the desired product as a colorless oil. To a solution of this intermediate (4.71 g, 16.47 mmol) in anhydrous THF (100 mL) cooled to 0° C. was added sodium hydride (0.99 g, 24.7 mmol, 60% dispersion). After 20 minutes, 2-[N,N-Bis(trifluoromethylsulfonyl)amino]-5-chloropyridine (7.76 g, 19.76 mmol) was added. The reaction was stirred at room temperature for 4 hours and then quenched with water. The resulting mixture was extracted with ethyl acetate (2×). The organic layer was washed with brine, dried over anhydrous sodium sulfate, filtered and concentrated in vacuo. The residue was purified by flash chromatography using 5% ethyl acetate-hexanes to give the desired product as a colorless oil. To a solution of this triflate intermediate (0.13 g, 0.31 mmol) and Intermediate A (0.090 g, 0.26 mmol) in anhydrous dioxane (3 mL) was added Xantphos (30 mg, 0.051 nm aol), cesium carbonate (117 mg, 0.36 mmol), then Pd₂(dba)₃ (23 mg, 0.026 mmol). The resulting mixture was stirred at 70° C. for 4.5 hours then left stirring at room temperature overnight. The reaction was filtered through a pad of celite. The celite was washed with ethyl acetate and dichloromethane. The filtrate and combined washes were concentrated in vacuo and purified by flash chromatography using a gradient of 0-30% ethyl acetate-hexanes over 10 column volumes, 30% ethyl acetate-hexanes for 6 column volumes, followed by a gradient of 30-100% ethyl acetate-hexanes over 7 column volumes. The desired product was isolated as a pale yellow solid. The individual stereoisomers were isolated at this intermediate by coupling the enantiomerically pure camino amides with the racemic triflate followed by chiral HPLC resolution of the resulting diastereomers. Two of the diastereomers prepared were resolved on a ChiralPak IA column using 7% EtOH-heptane to provide Isomer A as the faster eluting isomer after 70 minutes and Isomer B as the slower eluting isomer after 81 minutes. And two of the diastereomers were resolved on a ChiralPak OD-H column using 8% EtOH-heptane to provide Isomer C as the faster eluting isomer after 48 minutes and Isomer D as the slower eluting isomer after 55 minutes.

To a solution of intermediate Isomer D (29 mg, 0.046 mmol) in DCM (1 mL) at 0° C. was added triisopropylsilane (0.15 mL, 0.73 mmol) followed by TFA (0.5 mL). The reaction was stirred at room temperature for 1 hour then neutralized to pH=7 with saturated aqueous NaHCO₃. The resulting mixture was extracted with DCM (3×). The organic layers were dried over anhydrous sodium sulfate, filtered and concentrated in vacuo to give the product as a white solid. To a solution of this ester intermediate (29 mg) in dioxane (1.5 mL) at 0° C. was added 1N LiOH (1 mL). The mixture was stirred at room temperature for 2 hours. The reaction was quenched by the addition of 1N HCl (1 mL). The resulting mixture was extracted with ethyl acetate then DCM. The combined organic layers were dried over anhydrous sodium sulfate, filtered and concentrated in vacuo. The residue was purified by reverse phase HPLC (Gilson) to give the desired product as a white solid. ¹H NMR 8 (500 MHz, DMSO) 11.46 (s, 1H), 8.77 (d, 1H), 8.15 (dd, 1H), 7.95 (t, 1H), 7.41 (m, 1H), 7.11 (m, 1H), 4.58 (t, 1H), 3.60 (m, 2H, partially obscured by water), 3.16 (m, 1H), 3.09 (d, 1H), 2.77 (m, 1H), 2.46-2.36 (overlapping m, 2H), 1.86-1.75 (overlapping m, 2H); LCMS m/z 504 (M−H). Likewise all four isomers were prepared.

### Example 22

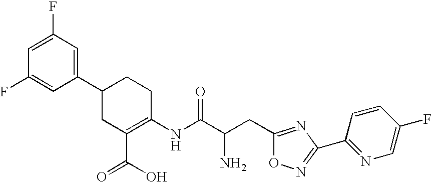

Standard access to the arylated beta-ketoester shown in Scheme 14 provides an intermediate that can be triflated. Thus to a solution of 1,4-cyclohexane dione mono-ethylene ketal (4.0 g, 25.61 mmol) in anhydrous THF (130 mL) cooled to −78° C. under a N₂ atmosphere was added LiHMDS (28 mL, 28 mmol, 1.0 M in THF). After stirring for 1 hour a solution 2-[N,N-Bis(trifluoromethylsulfonyl)amino]-5-chloropyridine (10.0 g, 25.46 mmol) in THF (100 mL) was added. The reaction was warmed to room temperature and stirred for 18 hours. The reaction was quenched with water and the resulting mixture was extracted with ethyl acetate(3×). The combined organic layers were washed with brine, dried over anhydrous sodium sulfate, filtered and concentrated in vacuo. The residue was purified by flash chromatography (Biotage, Horizon) using (0% EtOAc/Hexane+20% EtOAc/Hexane) to give the desired product as a colorless oil. To a solution of this intermediate triflate (1 eq) in THF was added the requisite boronic acid (1 eq), and tetrakis triphenyl phosphine palladium (0) (cat. 5%). Aqueous sodium carbonate solution (1M) was added, the reaction mixture was flushed with N₂ and heated to 50° C. for 1 hour. The mixture was cooled to room temperature, diluted with ethyl acetate, washed with brine, and dried over sodium sulfate. The crude material was purified by flash chromatography to give the desired product. To a solution of the olefinic ketal in MeOH was added palladium on carbon (5%) in MeOH. The reaction mixture was stirred under a hydrogen balloon for 18 hours, and then filtered through celite and concentrated in vacuo. The crude material was dissolved in THF/EtOH/3N HCl (5:2:4) was added. The resulting mixture was stirred at room temperature for 18 hours. The reaction mixture was concentrated in vacuo. The residue was diluted with ethyl acetate, and adjusted to pH=8 with 1 N NaOH. The resulting mixture was extracted with EtOAc (2×), washed with brine and dried over Na₂SO₄, filtered and concentrated in vacuo. The crude material was purified by flash chromatography to give the desired product. To a solution of this intermediate (1 eq) in anhydrous THF (61 mL) cooled to −78° C. under a N₂ atmosphere was added LiHMDS (1.5 eq, 1.0 M in TH). After 1 hour, methyl cyanoformate (1.4 eq) was added and the reaction mixture was allowed to warm to 40° C. over 2 hours. The mixture was quenched with 1N HCl and extracted with EtOAc (2×). The organic layer was washed with brine and dried over Na₂SO₄, filtered and concentrated in vacuo. This material was used in the next step without any further purification. The ketoester (347 mg, 0.93 mmol) was dissolved in anhydrous THF (10 mL). The mixture was cooled to 0° C. and treated with NaH (60%, 44 mg, 1.11 mmol). The ice bath is removed and warmed to room temperature over 30 minutes. At this point, Comins' reagent (369 mg, 0.927 mmol) is added and stirred overnight. The mixture is then quenched with 1N HCl (to pH 7) and extracted with EtOAC (2×). The organic phase is washed with brine and dried over Na₂SO₄, filtered and concentrated to yield a brown oil, which was purified byPTLC (10% EtOAc/hexane). This triflate (387 mg, 0.764 mmol), is combined with the enantiomerically pure carboxamide described in above examples (224 mg, 0.637 mmol), cesium carbonate (245 mg, 0.764 nmol), Xantphos (74 mg, 0.127 mmol) and anhydrous dioxane (6 mL). The reaction vessel was flushed with N₂ then treated with Pd₂ dba₃ (35 mg, 0.038 mmol) and the mixture heated to 75° C. overnight, cooled to room temperature then filtered through celite and concentrated, purified crude material by PTLC (30% EtOAc/hexane) and the separated enantiomers (at aryl stereocenter) was conducted by normal phase chiral SFC (ChiralPak IA, 25% IPA/CO₂). This protected intermediate (12 mg, first diastereomer to elute by chiral SFC) was dissolved in anhydrous CH₂Cl₂ (11 mL), treated with TFA (0.3 mL) and the mixture stirred overnight, cooled to 0° C. and then neutralized to pH 7 with saturated NaHCO₃ (aq), extracted with CH₂Cl₂(2×), washed with brine and dried over Na₂SO₄, filtered, and concentrated. The product was purified by reverse phase HPLC (10→100% MeCN/H₂O (1% TFA) to provide a final white powder. ¹H NMR (CD₃OD, 500 mHz), δ 8.68-8.67 (d, 1H), 8.30-8.27 (dd, 1H), 7.87-7.83 (m, 1H), 6.89-6.86 (m, 2H), 6.79-6.74 (m, 1H), 4.67-4.64 (m, 1H), 3.80-3.77 (m, 1H), 3.70-3.64 (m, 1H), 3.16-3.11 (m, 1H), 3.03-2.97 (m, 1H), 2.84-2.80 (m, 1H), 2.74-2.70 (m, 1H), 2.33-2.27 (m, 1H), 2.01-1.99 (m, 1H), 1.8-1.72 (m, 1H); LCMS m/z 488 (M+H).

### Example 23

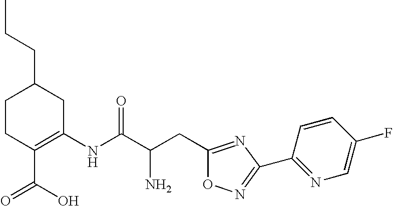

Propylmagnesium chloride (2 M in THF) was added to 3-ethoxy-2-cyclohexen-1-one (3.5 g, 25 mmol) in THF (100 mL) at 0° C. The reaction mixture was stirred overnight at room temperature and quenched with 1N HCl. This solution was washed twice with ethyl acetate and the combined organics were washed with brine and dried over sodium sulfate. Solvent was removed. At −78° C. LHMDS (32 mL, 32 mmol, 1.0 M in THF) was added to ketone in THF (100 mL). This was stirred at 0° C. for 40 minutes and then methyl cyanoformate (3 mL, 37 mmol) was added at −78° C. This reaction was then slowly warmed to room temperature and quenched with 1 N HCl. The solution was washed with ethyl acetate and the organic layer was washed with brine and dried over sodium sulfate. Solvent was removed and the residue was redissolved in MeOH (100 mL). The mixture was stirred under a balloon of hydrogen in the presence of 10% palladium on carbon (200 mg) overnight. The reaction mixture was filtered through celite and the solvent was removed. The ketoester was purified by flash chromatography using a 0-30% ethyl acetate/hexanes gradient. This ketoester (1.5 g, 7.6 mmol) was heated at reflux in 4-methoxylbenzyl alcohol (2.5 mL) and toluene (50 mL) for 24 h. Solvent was removed and product was purified by flash chromatography using a 0-30% ethyl acetate/hexanes gradient. Using methods described in previous examples, Example 23 was obtained. ¹H NMR (CD₃OD, 500 MHz) δ 8.68 (d, 1H), 8.28 (m, 1H), 7.85 (td, 1H), 4.63 (m, 1H), 3.71 (m, 1H), 3.08 (m, 2H), 2.52-2.39 (m, 2H), 2.26 (m, 1H), 1.79 (m, 1H), 1.37 (m, 2H), 1.31 (m, 3H), 1.18 (m, 1H), 0.93 (m, 3H). LCMS m/z 418 (M+H).

### Example 24

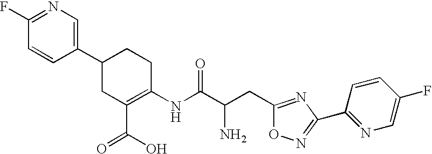

Example 24 was prepared under similar conditions described in the examples above. ¹H NMR (DMSO-d6, 500 MHz) δ 8.80 (d, 1H), 8.18-8.15 (m, 2H), 7.99 (m, 1H), 7.92 (m, 1H), 7.13 (m, 1H) 4.54 (m, 1H), 3.70-3.52 (m, 2H), 3.02-2.95 (m, 2H), 2.67-2.61 (m, 1H), 2.35-2.23 (m, 1H), 1.90 (m, 1H), 1.76 (m, 1H), 1.15 (m, 1H); LCMS m/z 471 (M+H).

### Example 25

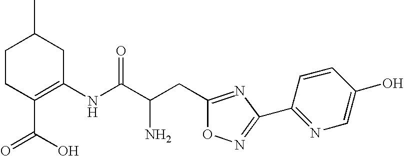

The N′-hydroxy-pyridinecarboximidamide intermediate for Example 25 was prepared according to an alternate procedure. Top-methoxybenzyl alcohol, in DMF (100 mL) at 0° C., was added sodium hydride (1.09 g, 46 mmol). The reaction mixture stirred for 30 minutes at room temperature, at which time, 5-bromo-2-cyanopyridine (7.1 g, 39 mmol) was added in portions. The mixture stirred for Ih and then was diluted with ethyl acetate (100 mL) and water (100 mL). The mixture was extracted with CH₂Cl₂ (100 mL), dried over sodium sulfate, concentrated in vacuo, and purified via flash chromatography (Biotage 40M). To the pyridine derivative (8.82 g, 37 mmol), in methanol (100 mL) at room temperature was added sodium bicarbonate (6.1 g, 73 mmol) and hydroxylamine-HCl (5.1 g, 73 mmol). The mixture was allowed to stir at room temperature for 24 h. The reaction mixture was filtered and the white solid was washed with chilled water and dried overnight. Once dry, the carboximidamide was used without further purification, toward the synthesis of Example 25. ¹H NMR (DMSO-d₆, 500 MHz) δ 11.56 (m, 1H), 8.25 (s, 1H), 7.93 (m, 1H), 7.33 (m, 1H), 4.55 (m, 1H), 3.5 (m, 2H), 2.83 (m, 2H), 2.24 (m, 2H), 1.60 (m, 2H), 1.13 (m, 1H), 0.96 (m, 3H); LCMS m/z 388 (M+H).

### Example 26

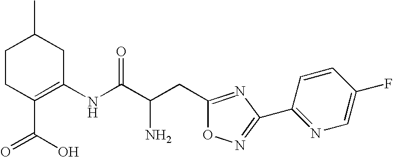

Example 26 was prepared under similar conditions described in the examples above. ¹H NMR (DMSO-d₆, 500 MHz) δ 11.56 (m, 1H), 8.78 (s, 1H), 8.17 (m, 1H), 7.98 (m, 1H), 4.54 (m, 1H), 3.63 (m, 2H), 2.88 (m, 2H), 2.33 (m, 2H), 1.65 (m, 2H), 1.15 (m, 1H), 1.12 (m, 3H); LCMS m/z 412 (M+Na).

### Example 27

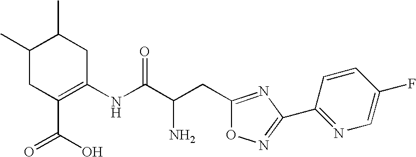

Example 27 was prepared under similar conditions described in the examples above. The 3,4-dimethylcyclohexanone starting material is commercially available as both the racemic-anti and racemic-syn isomers. For the anti-product of Example 27; ¹H NMR (DMSO-d₆, 500 MHz) δ 11.53 (m, 1H), 8.78 (s, 1H), 8.17 (m, 1H), 7.99 (m, 1H), 4.56 (m, 1H), 3.6 (m, 2H), 2.92 (m, 2H), 2.41 (m, 2H), 1.80 (m, 1H), 1.25 (m, 1H), 0.92 (m, 6H); LCMS m/z 404 (M+1). For the syn-product of Example 27; ¹H NMR (DMSO-d₆, 500 MHz) δ 11.58 (m, 1H), 8.77 (s, 1H), 8.15 (m, 1H), 7.97 (m, 1H), 4.53 (m, 1H), 3.61 (m, 2H), 2.81 (m, 2H), 2.49 (m, 2H), 1.97 (m, 1H), 1.79 (m, 1H), 0.88 (m, 6H); LCMS m/z 404 (M+H).

### Example 28

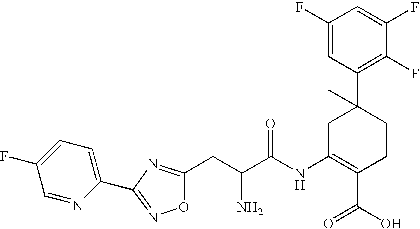

As illustrated in Scheme 16, to a solution of cyclohexane 1,3-dione (1.0 g, 8.92 mmol) and 2,6-lutidine (2.07 mL, 17.84 mmol) in DCM cooled to 0° C. was added trifluoromethane sulfonic anhydride (2.25 mL, 13.38 mmol). The reaction mixture was stirred at room temperature for 30 minutes and quenched by the addition of 1N HCl. The resulting mixture was extracted with DCM. The organic layer was washed with 1N HCl, dried over anhydrous sodium sulfate, filtered and concentrated in vacuo. The residue was purified by flash chromatography using 20% ethyl acetate hexanes to give the desired product a light brown oil. To a solution of this intermediate (1.0 g, 4.09 mmol) in THF (5 mL) was added phenyl boronic acid (749 mg, 6.13 mmol), Na₂CO₃ (3 ml, 1.0M solution) and dichlorobis(triphenylphosphine)palladium (144 mg, 0.2 nmol). After heating the reaction mixture at 50° C. for 30 minutes it was cooled to room temperature and diluted with ethyl acetate. The organic layer was washed with brine, dried over anhydrous sodium sulfate, filtered and concentrated in vacuo. The residue was purified by flash chromatography using 10% ethyl acetate-hexanes to give the desired compound as a white solid. To a suspension of copper(I) iodide (3.77 g, 19.8 mmol) in anhydrous diethyl ether (30 mL) cooled to 0° C. under a N₂ atmosphere was added drop-wise methyl lithium (24.8 mL, 39.6 mmol). After 15 minutes, the reaction mixture was cooled to −78° C. and a solution of the enone intermediate (0.69 g, 3.96 mmol) in ether (20 mL) was added. The reaction mixture was slowly warmed to room temperature and stirred for 1 hour. The mixture was quenched by the addition of saturated ammonium chloride solution. The resulting bi-phasic mixture was filtered through celite and washed extensively with ethyl acetate. The layers in the filtrate were separated and the aqueous layer extracted with ethyl acetate. The organic layer was washed with brine, dried over anhydrous sodium sulfate, filtered and concentrated in vacuo. The residue was purified by flash chromatography using 5% ethyl acetate hexanes to give the desired compound. To a solution of this intermediate (0.64 g, 3.36 mmol) in anhydrous THF (20 mL) cooled to −78° C. was added LHMDS (4 mL, 4.04 mmol, 1.0 M in THF). After 20 minutes, methyl cyanoformate (0.32 mL, 4.04 mmol) was added. The mixture was slowly warmed to −20° C. and quenched with 1N HCl. The resulting mixture was extracted with ethyl acetate (3×). The combined organic layers were washed with brine, dried over anhydrous sodium sulfate, filtered and concentrated in vacuo. The residue was purified by flash chromatography using 10% ethyl acetate-hexanes to give the desired product. To a solution of this intermediate (0.548 g, 2.22 mmol) in anhydrous THF (20 mL) cooled to 0° C. was added sodium hydride (0.133 g, 3.34 mmol, 60% by weight). After 30 minutes, 2-[N,N-Bis(trifluoromethylsulfonyl)amino]-5-chloropyridine (1.04 g, 2.66 mmol) was added. The reaction mixture was stirred at room temperature for two hours and then quenched with saturated ammonium chloride solution. The resulting mixture was extracted with ethyl acetate (3×). The combined organic layers were washed with brine, dried over anhydrous sodium sulfate, filtered and concentrated in vacuo. The residue was purified by flash chromatography using 2% then 5% ethyl acetate-hexanes to give the desired product as a colorless oil. Example 28 was prepared under these described conditions utilizing the appropriate 2,3,5-trifluorophenyl boronic acid, and similar procedures described in the examples above. ¹H NMR (DMSO-d₆, 500 MHz) δ 11.29 (s, 1H), 8.72 (m, 1H), 8.09 (m, 1H), 7.97 (m, 1H), 7.40 (m, 1H), 7.06 (m, 1H), 4.60 (m, 1H), 3.29 (m, 2H), 2.91 (m, 1H), 2.78 (m, 1H), 2.35 (m, 1H), 2.16 (m, 1H), 1.90 (m, 1H), 1.80 (m, 1H), 1.34 (m, 3H); LCMS m/z 542 (M+Na).

### Example 29

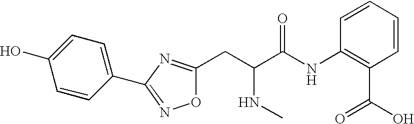

Example 29 was prepared directly from Example 5 via standard reductive amination conditions known to those skilled in the art, utilizing the solid trimeric form of paraformaldehyde. ¹H NMR (CD₃OD, 500 MHz) δ 8.49 (m, 1H), 8.12 (s, 1H), 7.87 (m, 2H), 7.63 (m, 1H), 7.27 (m, 1H), 6.78 (m, 2H), 4.75 (m, 1H), 3.79 (m, 2H), 2.64 (m, 3H); LCMS m/z 383 (M+H).

### Example 30

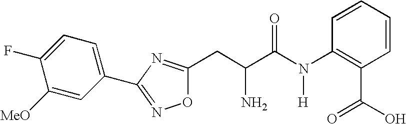

Example 30 was prepared under similar conditions described in the examples above and illustrated in Scheme 17. ¹H NMR (CD₃OD, 500 MHz) δ 8.53 (m, 1H), 8.13 (m, 1H), 7.7 (m, 2H), 7.25 (m, 3H), 4.79 (m, 1H), 3.90 (s, 3H), 3.77 (m, 2H); LCMS m/z 401 (M+H).

### Example 31

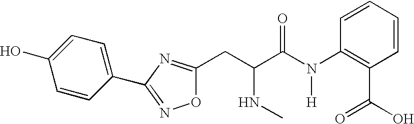

To the solution of ethyl-3-pyrazole carboxylate (3.53 g, 25.2 mmol) in DMF (40 mL) at 0° C. was added sodium hydride (60%, 1.21 g, 30.2 mmol). The resulting mixture was stirred at room temperature for 40 min followed by the addition of 5-nitro-2-bromopyridine (5.1 g, 25.2 mmol). After being stirred for 20 min, the reaction mixture was partitioned between dichloromethane (1000 mL) and water (500 mL), the organic phase was washed with water (3×500 mL), dried over sodium sulfate, and concentrated in vacuo. The residue was purified by flash chromatography using 80% DCM/hexane to give the desired biaryl product. To the solution of this nitro intermediate (6.77 g, 25.8 mmol) in acetic acid (220 mL) was added zinc powder (16.77 g, 258 mmol). The resulting mixture was heated at 60° C. for 30 min before it was filtered. The filtrate was concentrated in vacuum. To the residue was added DCM (1000 mL) and saturated sodium bicarbonate (1000 mL), and the resulting mixture was stirred at room temperature overnight. The organic phase was then washed with saturated sodium bicarbonate, dried over sodium sulfate, and concentrated in vacuo. The residue was purified by flash chromatography using 5% methanol in DCM (containing 0.1% triethylamine) to give the desired product as a yellow solid. To the solution of this aminopyridine (5.96 g, 25.7 mmol) in tetrafluoroboric acid (48%, 130 mL) at 0° C. was added a solution of sodium nitrite (1.95 g, 28.3 mmol) in water (20 mL) dropwise. The resulting solution was stirred at 0° C. for 1 h before filtration. The solid was washed with water and diethyl ether to give the desired product as a yellow solid. The mixture of diazo intermediate (6.66 g) in acetic anhydride (250 mL) was heated at 70° C. overnight before it was concentrated in vacuo. The residue was purified by flash chromatography eluting with DCM to give the desired product as a white solid. The solution of this acetate intermediate (3.5 g, 12.7 mmol) in ethanol (400 mL) in the presence of 4 drops of sulfuric acid was heated under reflux overnight. After being concentrated in vacuo, the residue was partitioned between DCM (300 mL) and water (200 mL). The pH of the resulting mixture was adjusted to pH=5 by saturated sodium bicarbonate solution. The DCM phase was dried with sodium sulfate and concentrated in vacuo to give the product hydroxypyridine as a solid. To this alcohol intermediate (2.86 g, 12.3 mmol) in DMF (40 mL) at 0° C. was added sodium hydride (60%, 589 mg, 14.73 mmol). The resulting mixture was stirred at room temperature for 40 min followed by adding 4-methoxybenzyl chloride (2.31 g, 14.73 mmol) and sodium iodide (10 mg). The resulting mixture was heated at 80° C. for 0.5 h. After being cooled to room temperature, the reaction mixture was partitioned between DCM (500 mL) and brine (500 mL). The DCM phase was washed with brine (3×500 mL), dried over sodium sulfate, and concentrated in vacuo. The residue was treated with 20% EtOAc/hexane (50 mL) and the mixture was filtered to give the desired product. The filtrate was concentrated and the resulting residue was purified by flash chromatography using 20% EtOAc/hexane to give additional product as a white solid. A suspension of this ester intermediate (4.13 g, 11.9 mmol) and lithium borohydride (384 mg, 17.6 mmol) in THF (300 mL) was heated under reflux overnight before it was cooled to 0° C. and quenched by 1N HCl until pH=6. The resulting mixture was diluted in EtOAc (400 mL) and washed with saturated sodium bicarbonate (2×400 mL), dried over sodium sulfate and concentrated in vacuo to give the desired product as a white solid. To a solution of this hydroxymethylene intermediate (3.7 g, 11.88 mmol) in DCM (200 mL) at 0° C. was added pyridine (1.13 g, 14.27 mmol), triphenylphosphine (8.73 g, 33.29 mmol) and NBS (6.34 g, 35.66 mmol). The resulting solution was stirred at 0° C. for 1.5 h. The DCM phase was washed with brine, dried over sodium sulfate and concentrated in vacuo. The residue was purified by flash chromatography eluting with DCM to give the product bromomethylene intermediate as a white solid. As shown in Scheme 19, Example 31 was prepared from this bromomethylene intermediate under conditions well-described in the literature and the examples above. ¹H NMR, (500 MHz, DMSO-d6): δ: 11.61(1H, s), 10.2 (1H, s), 8.38 (1H, s), 8.37 (3H, s), 7-95(1H, d), 7.71 (1H, m), 7.35 (1H, m), 6.37(1H, d), 4.30 (1H, m), 3.22 (2H, d), 2.72 (2H, m), 2.20 (2H, m), 1.52 (4H, m); LCMS m/z 372 (M+H).

### Example 32

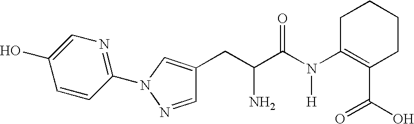

Example 32 was prepared from commercially available ethyl 4-pyrazolecarboxylate following similar conditions described in the examples above and illustrated in Scheme 20. ¹H NMR, (500 MHz, CD₃OD): δ: 8.36(1H, s), 7.96 (1H, s), 7.72 (1H, m), 7.59 (1H, s), 7.34 (1H, m), 4.20 (1H, m), 3.20 (2H, d), 2.92 (2H, m), 2.32 (2H, m), 1.62 (4H, m); LCMS m/z 372 (M+H).

### Example 33

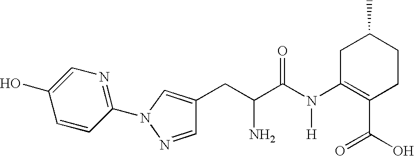

Example 33 was prepared under similar conditions described in the examples above, utilizing the commercially available (R)-3-methylcyclohexanone. ¹H NMR (CD₃OD-d₆, 500 MHz) δ 8.37 (1H, d), 7.97 (1H, s), 7.72 (1H, d), 7.60 (1H, d), 7.37 (1H, dd), 4.20 (1H, q), 3.32 (1H, s), 3.21 (2H, d), 3.05 (1H, m), 2.50 (2H, m), 2.22 (1H, m), 1.70 (2H, m), 1.02 (3H, d); LCMS m/z 386 (M+H).

### Biological Assays

The activity of the compounds of the present invention regarding niacin receptor affinity and function can be evaluated using the following assays:

### 3H-Niacin Binding Assay:

1. Membrane: Membrane preps are stored in liquid nitrogen in:
20 mM HEPES, pH 7.4
0.1 mM EDTA

Thaw receptor membranes quickly and place on ice. Resuspend by pipetting up and down vigorously, pool all tubes, and mix well. Use clean human at 15 μg/well, clean mouse at 10 ug/well, dirty preps at 30 ug/well.
1a. (human): Dilute in Binding Buffer.
1b. (human+4% serum): Add 5.7% of 100% human serum stock (stored at −20° C.) for a final concentration of 4%. Dilute in Binding Buffer.
1c. (mouse): Dilute in Binding Buffer.
   2. Wash buffer and dilution buffer: Make 10 liters of ice-cold Binding Buffer:
20 mM HEPES, pH 7.4
1 mM MgCl₂
0.01% CHAPS (w/v)
use molecular grade or ddH₂O water
   3. [5,6-³H]—nicotinic acid: American Radiolabeled Chemicals, Inc. (cat #ART-689). Stock is ˜50 Ci/mmol, 1 mCi/ml, 1 ml total in ethanol→20 μM

Make an intermediate ³H-niacin working solution containing 7.5% EtOH and 0.25 μM tracer. 40 μL of this will be diluted into 200 μL total in each well->1.5% EtOH, 50 nM tracer final.

### 4. Unlabeled Nicotinic Acid:

Make 100 nM, 10 mM, and 80 μM stocks; store at −20° C. Dilute in DMSO.

### 5. Preparing Plates:

1) Aliquot manually into plates. All compounds are tested in duplicate. 10 mM unlabeled nicotinic acid must be included as a sample compound in each experiment.
2) Dilute the 10 nM compounds across the plate in 1:5 dilutions (8 μl:40 μl).
3) Add 195 μL binding buffer to all wells of Intermediate Plates to create working solutions (250 μM→0). There will be one Intermediate Plate for each Drug Plate.
4) Transfer 5 μL from Drug Plate to the Intermediate Plate. Mix 4-5 times.

### 6. Procedure:

1) Add 140 μL of appropriate diluted 19CD membrane to every well. There will be three plates for each drug plate: one human, one human+serum, one mouse.
2) Add 20 μL of compound from the appropriate intermediate plate
3) Add 40 μL of 0.25 μM ³H-nicotinic acid to all wells.
4) Seal plates, cover with aluminum foil, and shake at RT for 34 hours, speed 2, titer plate shaker.
5) Filter and wash with 8×200 μL ice-cold binding buffer. Be sure to rinse the apparatus with >1 liter of water after last plate.
6) Air dry overnight in hood (prop plate up so that air can flow through).
7) Seal the back of the plate
8) Add 40 μL Microscint-20 to each well.
9) Seal tops with sealer.
10) Count in Packard Topcount scintillation counter.
11) Upload data to calculation program, and also plot raw counts in Prism, determining that the graphs generated, and the IC₅₀ values agree.

The compounds of the invention generally have an IC₅₀ in the ³H-nicotinic acid competition binding assay within the range of 1 nM to about 25 μM.

### ³⁵S-GTPγS Binding Assay:

Membranes prepared from Chinese Hamster Ovary (CHO)-K1 cells stably expressing the niacin receptor or vector control (7 μg/assay) were diluted in assay buffer (100 mM HEPES, 100 mM NaCl and 10 mM MgCl₂, pH 7.4) in Wallac Scintistrip plates and pre-incubated with test compounds diluted in assay buffer containing 40 μM GDP (final [GDP] was ˜10 μM) for 10 minutes before addition of ³⁵S-GTPγS to 0.3 nM. To avoid potential compound precipitation, all compounds were first prepared in 100% DMSO and then diluted with assay buffer resulting in a final concentration of 3% DMSO in the assay. Binding was allowed to proceed for one hour before centrifuging the plates at 4000 rpm for 15 minutes at room temperature and subsequent counting in a TopCount scintillation counter. Non-linear regression analysis of the binding curves was performed in GraphPad Prism.

### Membrane Preparation

### Materials:

CHO-K¹ cell culture medium: F-12 Kaighn's Modified Cell Culture Medium with 10% FBS, 2 mM L-Glutamine, 1 mM Sodium Pyruvate and 400 μg/ml G418

| | |
|---|---|
| Membrane Scrape Buffer: | 20 mM HEPES |
| | 10 mM EDTA, pH 7.4 |
| Membrane Wash Buffer: | 20 mM HEPES |
| | 0.1 mM EDTA, pH 7.4 |
| Protease Inhibitor Cocktail: | P-8340, (Sigma, St. Louis, MO) |

### Procedure:

(Keep everything on ice throughout prep; buffers and plates of cells)
Aspirate cell culture media off the 15 cm² plates, rinse with 5 mL cold PBS and aspirate.
Add 5 ml Membrane Scrape Buffer and scrape cells. Transfer scrape into 50 mL centrifuge tube. Add 50 uL Protease Inhibitor Cocktail.
Spin at 20,000 rpm for 17 minutes at 4° C.
Aspirate off the supernatant and resuspend pellet in 30 mL Membrane Wash Buffer. Add 50 μL Protease Inhibitor Cocktail.
Spin at 20,000 rpm for 17 minutes at 4° C.
Aspirate the supernatant off the membrane pellet. The pellet may be frozen at −80° C. for later use or it can be used immediately.

### Assay

### Materials:

Guanosine 5′-diphosphate sodium salt (GDP, Sigma-Aldrich Catalog #87127)
Guanosine 5′-[γ³⁵S] thiotriphosphate, triethylammonium salt ([³⁵S]GTPγS, Amersham Biosciences Catalog #SJ1320, ˜1000 Ci/mmol)
96 well Scintiplates (Perkin-Elmer #1450-501)
Binding Buffer: 20 mM HEPES, pH 7.4
   100 mM NaC₁₋₁₀
   mM MgCl₂
GDP Buffer: binding buffer plus GDP, ranging from 0.4 to 40 μM, make fresh before assay

### Procedure:

(total assay volume=100 μwell)

25 μL GDP buffer with or without compounds (final GDP 10 μM—so use 40 μM stock)

50 μL membrane in binding buffer (0.4 mg protein/mL)

25 μL [³⁵S]GTPγS in binding buffer. This is made by adding 5 μl [³⁵S]GTPγS stock into 10 mL binding buffer (This buffer has no GDP)
Thaw compound plates to be screened (daughter plates with 5 μL compound @ 2 mM in 100% DMSO)
Dilute the 2 mM compounds 1:50 with 245 μL GDP buffer to 40 μM in 2% DMSO. (Note: the concentration of GDP in the GDP buffer depends on the receptor and should be optimized to obtain maximal signal to noise; 40 μM).
Thaw frozen membrane pellet on ice. (Note: they are really membranes at this point, the cells were broken in the hypotonic buffer without any salt during the membrane prep step, and most cellular proteins were washed away)
Homogenize membranes briefly (few seconds—don't allow the membranes to warm up, so keep on ice between bursts of homogenization) until in suspension using a POLYTRON PT3100 (probe PT-DA 3007/2 at setting of 7000 rpm). Determine the membrane protein concentration by Bradford assay. Dilute membrane to a protein concentrations of 0.40 mg/ml in Binding Buffer. (Note: the final assay concentration is 20 μg/well).
Add 25 μL compounds in GDP buffer per well to Scintiplate.
Add 50 μL of membranes per well to Scintiplate.
Pre-incubate for 5-10 minutes at room temperature. (cover plates with foil since compounds may be light sensitive)
Add 25 μL of diluted [³⁵S]GTPγS. Incubate on shaker (Lab-Line model #1314, shake at setting of 4) for 60 minutes at room temperature. Cover the plates with foil since some compounds might be light sensitive.
Assay is stopped by spinning plates sealed with plate covers at 2500 rpm for 20 minutes at 22° C.
Read on TopCount NXT scintillation counter—35S protocol.

The compounds of the invention generally have an EC₅₀ in the functional in vitro GTPγS binding assay within the range of about less than 1 μM to as high as about 100 μM.

### Flushing Via Laser Dopler

Male C57B16 mice (˜25 g) are anesthetized using 10 mg/ml/kg Nembutal sodium. When antagonists are to be administered they are co-injected with the Nembutal anesthesia. After ten minutes the animal is placed under the laser and the ear is folded back to expose the ventral side. The laser is positioned in the center of the ear and focused to an intensity of 8.4-9.0 V (with is generally ˜4.5 cm above the ear). Data acquisition is initiated with a 15 by 15 image format, auto interval, 60 images and a 20 sec time delay with a medium resolution. Test compounds are administered following the 10th image via injection into the peritoneal space. Images 1-10 are considered the animal's baseline and data is normalized to an average of the baseline mean intensities.

Materials and Methods—Laser Doppler Pirimed PimII; Niacin (Sigma); Nembutal (Abbott labs).

All patents, patent applications and publications that are cited herein are hereby incorporated by reference in their entirety. While certain preferred embodiments have been described herein in detail, numerous alternative embodiments are seen as falling within the scope of the invention.

1. A compound represented by formula I: 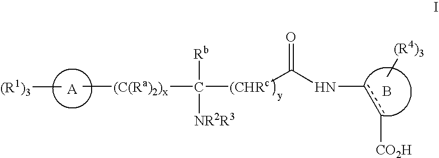
or a pharmaceutically acceptable salt, solvate or ester thereof wherein:
ring A represents a 6-10 membered aryl, a 5-13 membered heteroaryl or a non-aromatic or partially aromatic heterocyclic group, said heteroaryl and non-aromatic and partially aromatic heterocyclic groups containing at least one heteroatom selected from O, S, S(O), S(O)₂ and N, and optionally containing 1 other heteroatom selected from O and S, and optionally containing 1-3 additional N atoms, with up to 5 heteroatoms being present;
ring B represents a phenyl, thiophene or a cyclohexenyl ring in which the dotted line and the line which it is adjacent to represent in combination a double bond;
each R¹ is H or is independently selected from the group consisting of:
a) halo, OH, CO₂H, CN, NH₂, S(O)₀₋₂R^{e}, C(O)R^{e}, OC(O)R^{e} and CO₂R^{e}, wherein R^{e} represents C₁₋₄alkyl or phenyl, said C₁₋₄alkyl and phenyl each being optionally substituted with 1-3 groups, 1-3 of which are selected from halo and C₁₋₃alkyl, and 1-2 of which are selected from the group consisting of: OC₁₋₃alkyl, haloC₁₋₃alkyl, haloC₁₋₃alkoxy, OH, NH₂ and NHC₁₋₃alkyl;
b) C₁₋₆ alkyl and OC₁₋₆alkyl, said C₁₋₆alkyl and alkyl portion of OC₁₋₆-alkyl being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy and CN;
c) NHC₁₋₄alkyl and N(C₁₋₄alkyl)₂, the alkyl portions of which are optionally substituted as set forth in (b) above;
d) C(O)NH₂, C(O)NHC₁₋₄alkyl, C(O)N(C₁₋₄alkyl)₂, C(O)Hetcy, C(O)NHOC₁₋₄alkyl and C(O)N(C₁₋₄alkyl)(OC₁₋₄alkyl), the alkyl portions of which are optionally substituted as set forth in (b) above;
e) NR′C(O)R″, NR′SO₂R″, NR′CO₂R″ and NR′C(O)NR″R′″ wherein:
R′ represents H, C₁₋₃alkyl or haloC₁₋₃alkyl,
R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄haloalkyl, NH₂, NHC₁₋₄alkyl, N(C₁₋₄alkyl)₂, CN, Hetcy, Aryl and HAR,
said Hetcy, Aryl and HAR being further optionally substituted with 1-3 halo, C₁₋₄alkyl, C₁₋₄alkoxy, haloC₁₋₄alkyl or haloC₁₋₄alkoxy groups; and
(b) Hetcy, Aryl or HAR, each being optionally substituted with 1-3 members selected from the group consisting of: halo, C₁₋₄alkyl, C₁₋₄alkoxy, haloC₁₋₄alkyl and haloC₁₋₄alkoxy groups;
and R′″ representing H or R″;
f) phenyl or a 5-6 membered heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH; CO₂H; CN; NH₂ and S(O)₀₋₂R^{e} wherein R^{e} is as described above;
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, NH₂, NHC₁₋₄alkyl, N(C₁₋₄alkyl)₂ and CN;
iii) C(O)NH₂, C(O)NHC₁₋₄alkyl, C(O)N(C₁₋₄alkyl)₂, C(O)NHOC₁₋₄alkyl and C(O)N(C₁₋₄alkyl)(OC₁₋₄alkyl), the alkyl portions of which are optionally substituted as set forth in b) above; and
iv) NR′C(O)R″, NR′SO₂R″, NR′CO₂R″ and NR″C(O)NR″R′″ wherein R′, R″ and R′″ are as described above;
one of x and y is 0 and the other is 1;
each R^{a}, R^{b} and R^{c} are selected from H, C₁₋₃alkyl and haloC₁₋₃alkyl;
R² and R³ represent H, C₁₋₃alkyl or haloC₁₋₃alkyl;
3 R⁴ groups are present, 0-1 of which represents Aryl, HAR or Hetcy, said Aryl, HAR or Hetcy group being optionally substituted with up to 3 groups, 1-3 of which are halo, and 0-1 of which are selected from the group consisting of: OH, NH₂, C₁₋₃alkyl, C₁₋₃alkoxy, haloC₁₋₃alkyl and haloC₁₋₃alkoxy;
and the remainder of the R⁴groups are selected from the group consisting of: H, halo, C₁₋₃alkyl, C₁₋₃alkoxy, OH, NH₂, NHC₁₋₃alkyl, N(C₁₋₃alkyl)₂ and CN, said alkyl and alkyl portions of C₁₋₃alkoxy, NHC₁₋₃alkyl and N(C₁₋₃alkyl)₂ being optionally substituted with 1-3 groups, 0-3 of which are halo, and 0-1 of which are selected from the group consisting of: OC₁₋₃alkyl, OH, NH₂, NHC₁₋₃alkyl, N(C₁₋₃alkyl)₂, CN, Hetcy, Aryl and HAR,
said Aryl and HAR being further optionally substituted with 1-3 groups, 0-3 of which are halo, and 0-1 of which are selected from the group consisting of: OH, NR₂, C₁₋₃alkyl, C₁₋₃alkoxy, haloC₁₋₃alkyl and haloC₁₋₃alkoxy groups.

2. A compound represented by formula Ia: 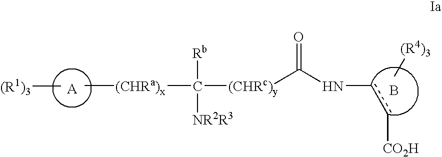
or a pharmaceutically acceptable salt, solvate or ester thereof wherein:
ring A represents a 6-10 membered aryl, a 5-13 membered heteroaryl or a non-aromatic or partially aromatic heterocyclic group, said heteroaryl and non-aromatic and partially aromatic heterocyclic groups containing at least one heteroatom selected from O, S, S(O), S(O)₂ and N, and optionally containing 1 other heteroatom selected from O and S, and optionally containing 1-3 additional N atoms, with up to 5 heteroatoms being present;
ring B represents a phenyl, thiophene or a cyclohexenyl ring in which the dotted line and the line which it is adjacent to represent in combination a double bond;
each R′ is H or is independently selected from the group consisting of:
a) halo, OH, CO₂H, CN, NH₂, S(O)₀₋₂R^{e}, C(O)R^{e}, OC(O)R^{e} and CO₂R^{e}, wherein R^{e} represents C₁₋₄alkyl or phenyl, said C₁₋₄alkyl and phenyl each being optionally substituted with 1-3 groups, 1-3 of which are selected from halo and C₁₋₃alkyl, and 1-2 of which are selected from the group consisting of: OC₁₋₃alkyl, haloC₁₋₃alkyl, haloC₁₋₃alkoxy, OH, NH₂ and NHC₁₋₃alkyl;
b) C₁— alkyl and OC₁₋₆alkyl, said C₁₋₆alkyl and alkyl portion of OC₁₋₆alkyl being optionally substituted with 1-3 groups, 1-3 of which are halo and 1-2 of which are selected from: OH, CO₂H, CO₂C₁₋₄alkyl, CO₂C₁₋₄haloalkyl, OCO₂C₁₋₄alkyl, NH₂, NHC₁₋₄alkyl, N(C₁₋₄alkyl)₂, Hetcy and CN;
c) NHC₁₋₄alkyl and N(C₁₋₄alkyl)₂, the alkyl portions of which are optionally substituted as set forth in (b) above;
d) C(O)NH₂, C(O)NHC₁₋₄alkyl, C(O)N(C₁₋₄alkyl)₂, C(O)Hetcy, C(O)NHOC₁₋₄alkyl and C(O)N(C₁₋₄alkyl)(OC₁₋₄alkyl), the alkyl portions of which are optionally substituted as set forth in (b) above;
e) NR′C(O)R″, NR′SO₂R″, NR′CO₂R″ and NR′C(O)NR″R′″ wherein:
R′ represents H, C₁₋₃alkyl or haloC₁₋₃alkyl,
R″ represents (a) C₁₋₈alkyl optionally substituted with 1-4 groups, 0-4 of which are halo, and 0-1 of which are selected from the group consisting of: OC₁₋₆alkyl, OH, CO₂H, CO₂C₁₋₄alkyl, CO₂C₁₋₄haloalkyl, NH₂, NHC₁₋₄alkyl, N(C₁₋₄alkyl)₂, CN, Hetcy, Aryl and HAR,
said Hetcy, Aryl and HAR being further optionally substituted with 1-3 halo, C₁₋₄alkyl, C₁₋₄alkoxy, haloC₁₋₄alkyl or haloC₁₋₄alkoxy groups; and
(b) Hetcy, Aryl or HAR, each being optionally substituted with 1-3 members selected from the group consisting of: halo, C₁₋₄alkyl, C₁₋₄alkoxy, haloC₁₋₄alkyl and haloC₁₋₄alkoxy groups;
and R′″ representing H or R″;
f) phenyl or a 5-6 membered heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH; CO₂H; CN; NH₂ and S(O)₀₋₂R^{e} wherein R^{e} is as described above;
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, NH₂, NHC₁₋₄alkyl, N(C₁₋₄alkyl)₂ and CN;
iii) C(O)NH₂, C(O)NHC₁₋₄alkyl, C(O)N(C₁₋₄alkyl)₂, C(O)NHOC₁₋₄alkyl and C(O)N(C₁₋₄alkyl)(OC₁₋₄alkyl), the alkyl portions of which are optionally substituted as set forth in b) above; and
iv) NR′C(O)R″NR′SO₂R″, NR′CO₂R″ and NR′C(O)NR″R′″ wherein R′, R″ and R′″ are as described above;
one of x and y is 0 and the other is 1;
R^{a}, R^{b} and R^{c} are selected from H, C₁₋₃alkyl and haloC₁₋₃alkyl;
R² and R³ represent H, C₁₋₃alkyl or haloC₁₋₃alkyl;
3 R⁴ groups are present, 0-1 of which represents Aryl, HAR or Hetcy, said Aryl, HAR or Hetcy group being optionally substituted with up to 3 groups, 1-3 of which are halo, and 0-1 of which are selected from the group consisting of: OH, NH₂, C₁₋₃alkyl, C₁₋₃alkoxy, haloC₁₋₃alkyl and haloC₁₋₃alkoxy;
and the remainder of the R⁴groups are selected from the group consisting of: H, halo, C₁₋₃alkyl, C₁₋₃alkoxy, OH, NH₂, NHC₁₋₃alkyl, N(C₁₋₃alkyl)₂ and CN, said alkyl and alkyl portions of C₁₋₃alkoxy, NHC₁₋₃alkyl and N(C₁₋₃alkyl)₂ being optionally substituted with 1-3 groups, 0-3 of which are halo, and 0-1 of which are selected from the group consisting of: OC₁₋₃alkyl, OH, NH₂, NHC₁₋₃alkyl, N(C₁₋₃alkyl)₂, CN, Hetcy, Aryl and HAR,
said Aryl and HAR being further optionally substituted with 1-3 groups, 0-3 of which are halo, and 0-1 of which are selected from the group consisting of: OH, NH₂, C₁₋₃alkyl, C₁₋₃alkoxy, haloC₁₋₃alkyl and haloC₁₋₃alkoxy groups.

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. A compound in accordance with claim 1 wherein each R¹ is H or is independently selected from the group consisting of:
(a) halo, OH, CO₂H, CN, NH₂, S(O)₀₋₂R^{e}, C(O)R^{e}, OC(O)R^{e} and CO₂R^{e}, wherein R^{e} represents C₁₋₄alkyl or phenyl, said C₁₋₄alkyl and phenyl each being optionally substituted with 1-3 groups, 1-3 of which are selected from halo and C₁₋₃alkyl, and 1-2 of which are selected from the group consisting of: OC₁₋₃alkyl, haloC₁₋₃alkyl, haloC₁₋₃alkoxy, OH, NH₂ and NHC₁₋₃alkyl; and
(b) phenyl or a 5-6 membered heteroaryl or a Hetcy group attached at any available ring atom and each being optionally substituted with 1-3 groups, 1-3 of which are selected from halo, C₁₋₃alkyl and haloC₁₋₃alkyl groups, and 1-2 of which are selected from OC₁₋₃alkyl and haloOC₁₋₃alkyl groups, and 0-1 of which is selected from the group consisting of:
i) OH; CO₂H; CN; NH₂ and S(O)₀₋₂R^{e} wherein R^{e} is as described above; and
ii) NHC₁₋₄alkyl and N(C₁₋₄alkyl)₂, the alkyl portions of which are optionally substituted with 1-3 groups, 1-3 of which are halo and 1-2 of which are selected from: OH, CO₂H, CO₂C₁₋₄alkyl, CO₂C₁₋₄haloalkyl, NH₂, NHC₁₋₄alkyl, N(C₁₋₄alkyl)₂ and CN.

32. (canceled)

33. (canceled)

34. (canceled)

35. (canceled)

36. (canceled)

37. (canceled)

38. A compound in accordance with claim 1 selected from the group consisting of:
TABLE I

| | |
|---|---|
| | COMPOUND 1 |
| 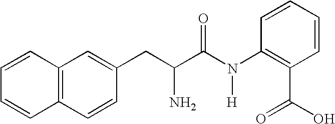 | |
| | COMPOUND 2 |
| 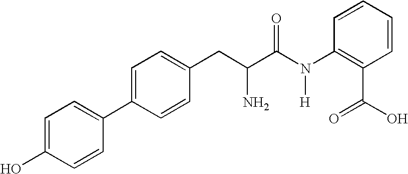 | |
| | COMPOUND 3 |
| 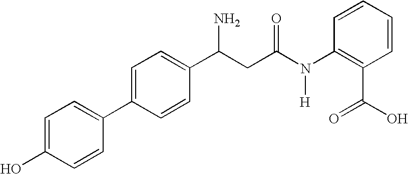 | |
| | COMPOUND 4 |
| 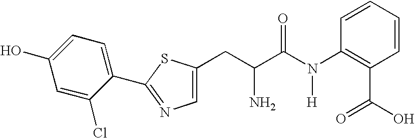 | |
| | COMPOUND 5 |
| 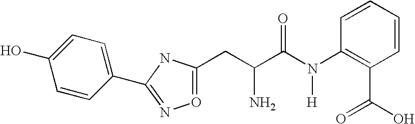 | |
| | COMPOUND 6 |
| 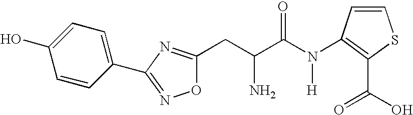 | |
| | COMPOUND 7 |
| 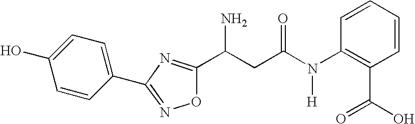 | |
| | COMPOUND 8 |
| 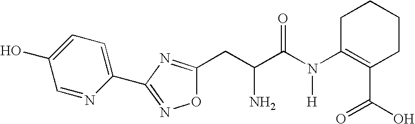 | |
| | COMPOUND 9 |
| 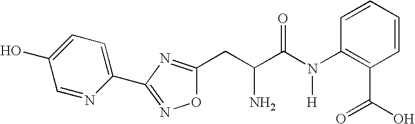 | |
| | COMPOUND 10 |
| 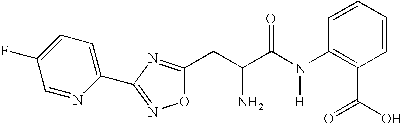 | |
| | COMPOUND 11 |
| 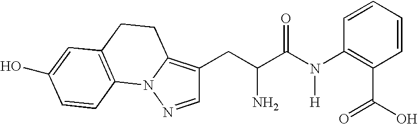 | |
| | COMPOUND 12 |
| 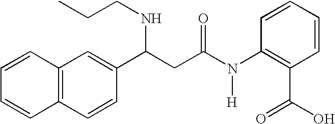 | |
| | COMPOUND 13 |
| 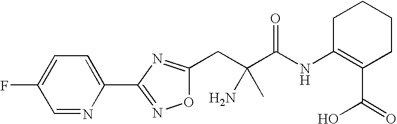 | |
| | COMPOUND 14 |
| 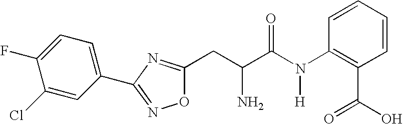 | |
| | COMPOUND 15 |
| 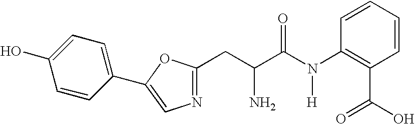 | |
| | COMPOUND 16 |
| 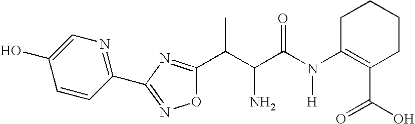 | |
| | COMPOUND 17 |
| 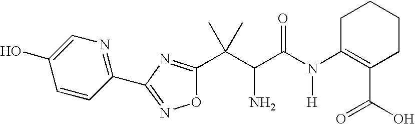 | |
| | COMPOUND 18 |
| 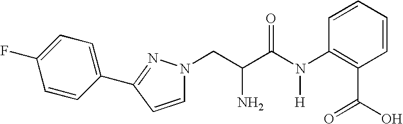 | |
| | COMPOUND 19 |
| 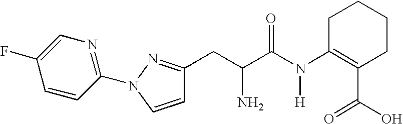 | |
| | COMPOUND 20 |
| 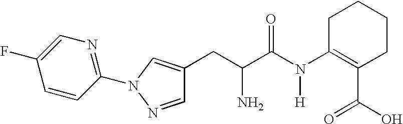 | |
| | COMPOUND 21 |
| 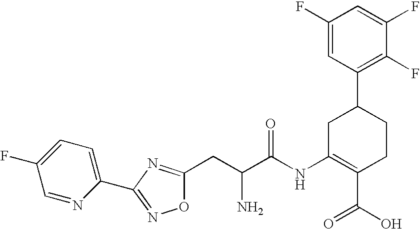 | |
| | COMPOUND 22 |
| 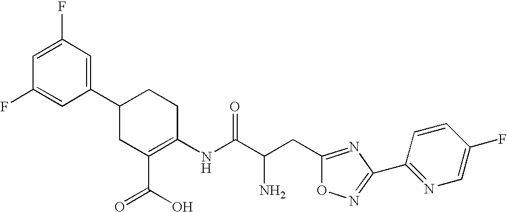 | |
| | COMPOUND 23 |
| 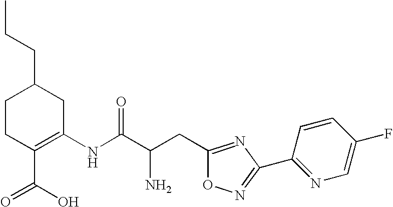 | |
| | COMPOUND 24 |
| 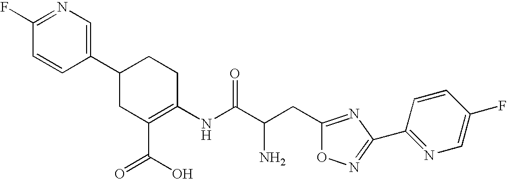 | |
| | COMPOUND 25 |
| 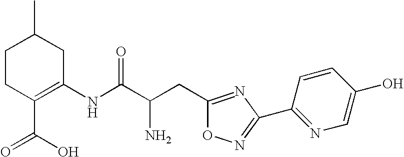 | |
| | COMPOUND 26 |
| 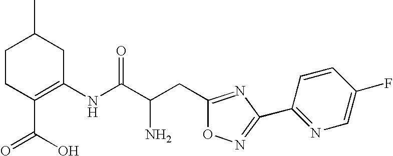 | |
| | COMPOUND 27 |
| 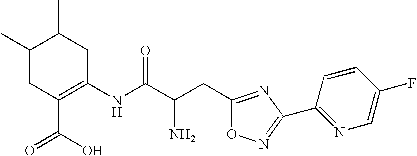 | |
| | COMPOUND 28 |
| 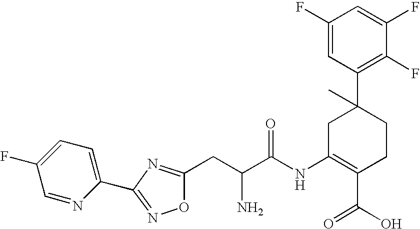 | |
| | COMPOUND 29 |
| 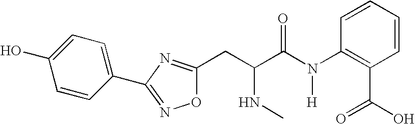 | |
| | COMPOUND 30 |
| 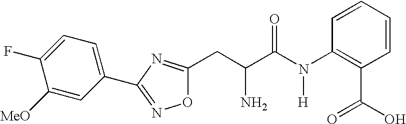 | |
| | COMPOUND 31 |
| 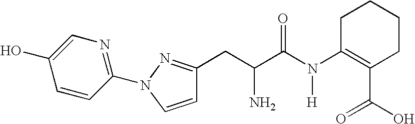 | |
| | COMPOUND 32 |
| 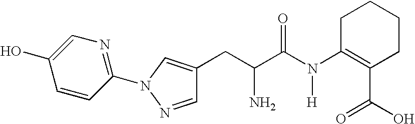 | |
| | COMPOUND 33 |
| 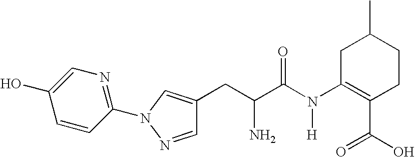 | |

and the pharmaceutically acceptable salts and solvates thereof.

39. A pharmaceutical composition comprising a compound in accordance with claim 1 in combination with a pharmaceutically acceptable carrier.

40. A method of treating atherosclerosis in a human patient in need of such treatment comprising administering to the patient a compound of claim 1 in an amount that is effective for treating atherosclerosis.

41. A method of treating dyslipidemia in a human patient in need of such treatment comprising administering to the patient a compound of claim 1 in an amount that is effective for treating dyslipidemias.

42. A method of treating diabetes in a human patient in need of such treatment comprising administering to the patient a compound of claim 1 in an amount that is effective for treating diabetes.

43. A method of treating metabolic syndrome in a human patient in need of such treatment comprising administering to the patient a compound of claim 1 in an amount that is effective for treating metabolic syndrome.

44. A method of treating atherosclerosis, dyslipidemias, diabetes, metabolic syndrome or a related condition in a human patient in need of such treatment, comprising administering to the patient a compound of claim 1 and a DP receptor antagonist, said compounds being administered in an amount that is effective to treat atherosclerosis, dyslipidemia, diabetes or a related condition in the absence of substantial flushing.

45. A method in accordance with claim 44 wherein the DP receptor antagonist selected from the group consisting of compounds A through AJ:

| | |
|---|---|
| | Compound A |
| 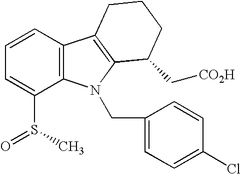 | |
| | Compound B |
| 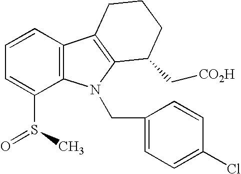 | |
| | Compound C |
| 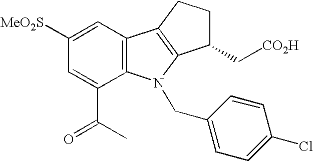 | |
| | Compound D |
| 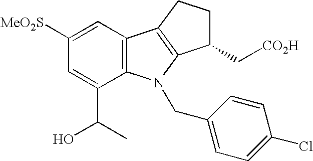 | |
| | Compound E |
| 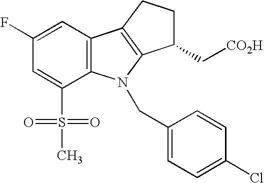 | |
| | Compound F |
| 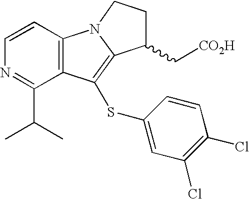 | |
| | Compound G |
| 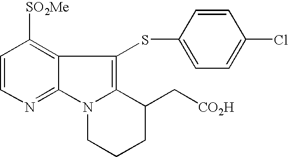 | |
| | Compound H |
| 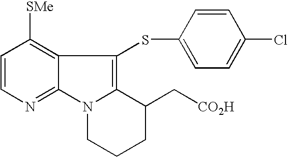 | |
| | |
|---|---|
| | Compound I |
| 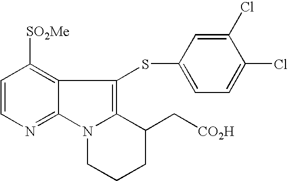 | |
| | Compound J |
| 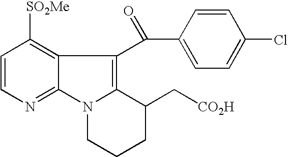 | |
| | |
|---|---|
| | Compound K |
| 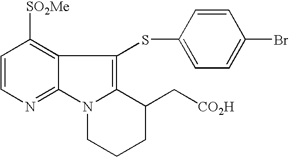 | |
| | Compound L |
| 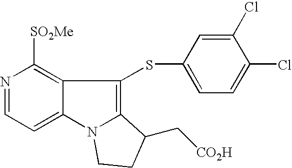 | |
| | Compound M |
| 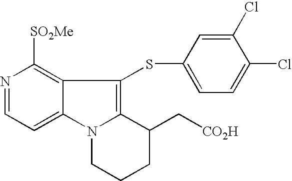 | |
| | Compound N |
| 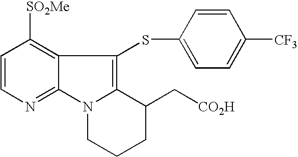 | |
| | Compound O |
| 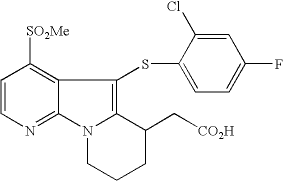 | |
| | Compound P |
| 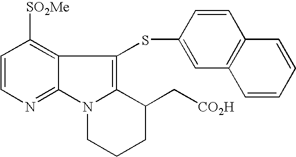 | |
| | Compound Q |
| 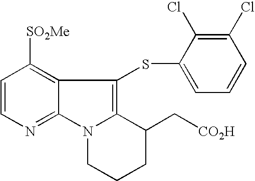 | |
| | Compound R |
| 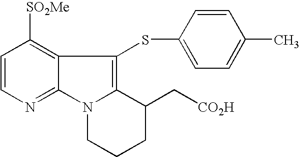 | |
| | Compound S |
| 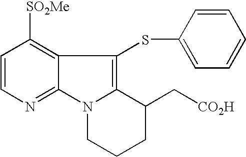 | |
| | |
|---|---|
| | Compound T |
| 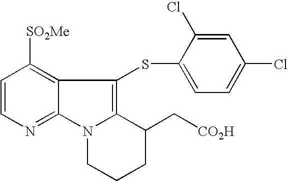 | |
| | |
|---|---|
| | Compound U |
| 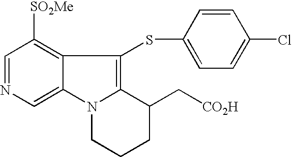 | |
| | Compound V |
| 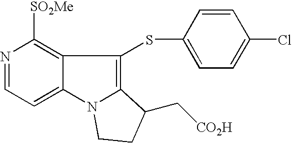 | |
| | Compound W |
| 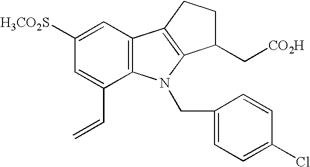 | |
| | Compound X |
| 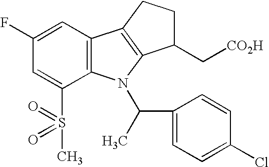 | |
| | Compound Y |
| 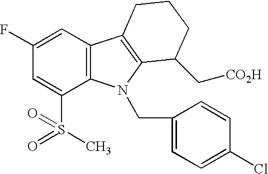 | |
| | Compound Z |
| 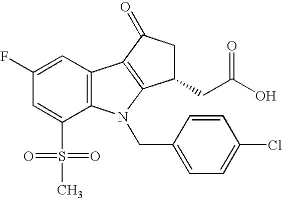 | |
| | Compound AA |
| 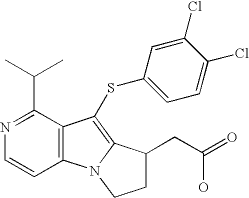 | |
| | Compound AB |
| 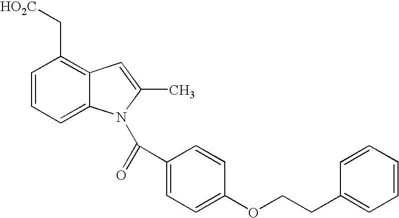 | |
| | Compound AC |
| 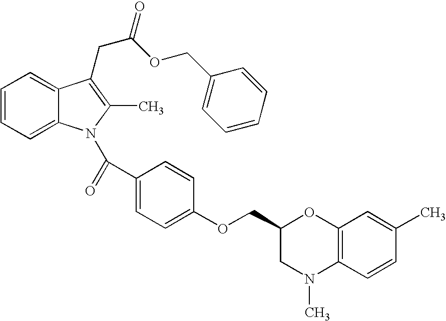 | |
| | Compound AD |
| 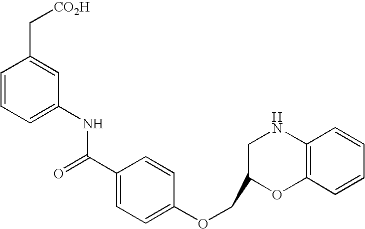 | |
| | Compound AE |
| 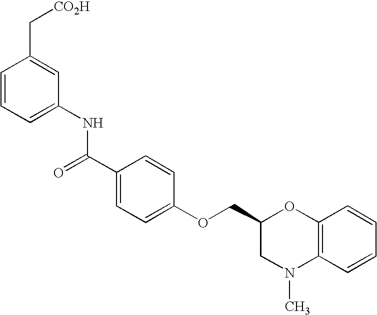 | |
| | Compound AF |
| 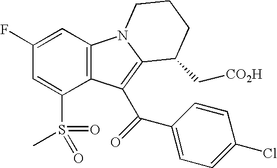 | |
| | Compound AG |
| 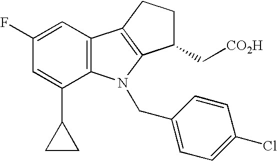 | |
| | Compound AH |
| 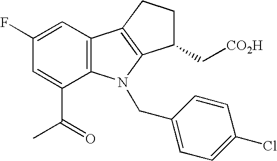 | |
| | Compound AI |
| 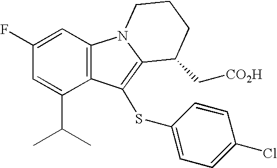 | |
| | Compound AJ |
| 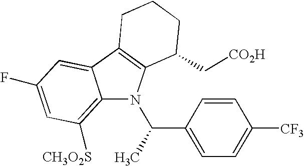 | |

or a pharmaceutically acceptable salt or solvate thereof.
